(19) United States
(12) Patent Application Publication
(10) Pub. No.: US 2024/0059755 A1
(43) Pub. Date: Feb. 22, 2024

(54) NANOLIPOPROTEIN-POLYPEPTIDE CONJUGATES AND COMPOSITIONS, SYSTEMS, AND METHODS USING SAME

(71) Applicant: Genentech, Inc., South San Francisco, CA (US)

(72) Inventors: Whitney BINDER, Pacifica, CA (US); Craig D. BLANCHETTE, San Bruno, CA (US); Martine A. DARWISH, San Francisco, CA (US); Rami HANNOUSH, San Mateo, CA (US); Xinxin GAO, San Francisco, CA (US)

(73) Assignee: Genentech, Inc, South San Francisco, CA (US)

(21) Appl. No.: 18/078,556

(22) Filed: Dec. 9, 2022

Related U.S. Application Data
(63) Continuation of application No. PCT/US21/36878, filed on Jun. 10, 2021.
(60) Provisional application No. 63/038,075, filed on Jun. 11, 2020.
(60) Provisional application No. 63/151,591, filed on Feb. 19, 2021.

(51) Int. Cl. C07K 14/775; C07K 16/28

(52) U.S. Cl. CPC C07K 14/775; C07K 16/2875; C07K 2317/55; C07K 2319/30; C07K 2319/31

(57) ABSTRACT

The present invention generally relates to nanolipoprotein particle conjugates comprising at least one polypeptide and nanolipoprotein particle conjugates comprising at least one short peptide between 20-60 amino acids in length. Also provided are related compositions and systems, and methods of preparing and using same. In particular, nanolipoprotein particle (NLP) conjugates with antigen-binding fragments (Fabs) are provided, where the NLP comprises a bilayer of membrane-forming lipids encircled by scaffold proteins, and the Fabs are conjugated to one or both bilayer surfaces. In some embodiments, the (NLP) conjugates further comprise a short peptide 20-60 amino acids in length. The conjugates, and related compositions and systems, generally show good stability and manufacturability, retain antigen-binding activity, and can enhance antigen-binding potency, providing versatile platforms for delivery of Fab and Fab-based therapeutics and diagnostics, as well as providing stable platforms for delivering other therapeutic and/or diagnostic agents.

### CROSS REFERENCE TO RELATED APPLICATIONS

This application is continuation application of International Application No. PCT/US2021/036878, filed internationally on Jun. 10, 2021, which claims the priority benefit of U.S. Provisional Application 63/038,075, filed Jun. 11, 2020; U.S. Provisional Application 63/151,591, filed Feb. 19, 2021, the contents of each of which are incorporated herein by reference in their entirety.

### REFERENCE TO AN ELECTRONIC SEQUENCE LISTING

The contents of the electronic sequence listing (146392052800SEQLIST.xml; Size: 2,001 bytes; and Date of Creation: Aug. 15, 2023) is herein incorporated by reference in its entirety.

### FIELD OF THE INVENTION

The present invention generally relates to nanolipoprotein particle conjugates comprising at least one conjugated polypeptide (e.g., covalently conjugated polypeptide), related compositions and systems, and methods of preparing and using same. In particular, nanolipoprotein particle (NLP) conjugates with antigen-binding fragments (Fabs) are provided, where the NLP comprises a bilayer of membrane-forming lipids encircled by scaffold proteins, and the Fabs are conjugated to one or both bilayer surfaces. The NLP-Fab conjugates, and related compositions and systems, generally show good stability and manufacturability, retain antigen-binding activity, and can enhance antigen-binding potency, providing versatile platforms for delivery of Fab and Fab-based therapeutics and diagnostics, as well as providing stable platforms for delivering other therapeutic and/or diagnostic agents. The present invention also relates to nanolipoprotein particle conjugates comprising at least one conjugated peptide, e.g., in addition to the conjugated polypeptide, or instead of the conjugated polypeptide.

### BACKGROUND

Over the past ten years, advances in the field of nanotechnology have sought to address limitations of conventional drug delivery systems. A range of nanoparticles has also been developed over the past several decades, including inorganic nanoparticles, polymeric based nanoparticles, polymeric micelles, dendrimers, liposomes, viral nanoparticles, carbon nanotubes, and nanolipoprotein particles (NLPs).

NLPs are nanometer-sized particles that generally consist of amphipathic lipids and apolipoprotein that self-assemble to form a discoidal lipid bilayer, where the hydrophobic periphery of the disc is stabilized by binding to the apolipoprotein. NLPs have been explored for select applications and drug cargo. Nonetheless, no previous studies have evaluated this platform for delivery of fragment antibodies (Fabs).

There thus remains a need in the art for novel NLP delivery platforms, in particular, stable compositions and systems, with good manufacturability, for delivering antigen-binding polypeptides and/or for targeting other therapeutic and diagnostic agents. The present invention meets these and other needs.

### SUMMARY OF THE INVENTION

The present invention generally relates to nanolipoprotein-polypeptide conjugates and compositions, systems, and methods of making and using the conjugates, where a nanolipoprotein particle is conjugated to polypeptides such as an antigen-binding polypeptide (e.g., a Fab) or a cysteine knot peptide (CKP), to give conjugates, with surprising properties in some embodiments. Conjugates of the present invention can provide advantages and improvements, such as low toxicity, low immunogenicity, good stability (without crosslinkers), good manufacturability (including feasible production of highly concentrated but low viscosity formulations), minimal to no inter-particle crosslinking, a relatively homogeneous NLP population, and/or enhanced antigen-binding potency.

In one aspect, the invention provides a conjugate comprising a scaffold protein, a membrane-forming lipid, and an antigen-binding polypeptide, where the membrane-forming lipids arrange in a lipid bilayer and the scaffold protein encircles the bilayer; and where the antigen-binding polypeptide is conjugated to one or more of the membrane-forming lipids on one or both surfaces of the bilayer via a functionalized group on one or more membrane-forming lipids and a complementary functional group located C-terminally on the antigen-binding polypeptide. In some embodiments, the antigen-binding polypeptide is a Fab. In some embodiments, a spacer connects the functionalized groups to the membrane-forming lipid and/or a spacer connects the complementary functional group to the antigen-binding polypeptide. In some embodiments, the spacer is GSGS.

In some embodiments, the functionalized group is selected from the group consisting of a maleimide derivative, a haloacetamide, a pyridyldithio-propionate, a thiosulfate, and a combination of any one or more thereof; and, in some embodiments, the complementary functional group is a free thiol group, such as a cysteine thiol group, optionally where the cysteine forms a hinge disulfide bond in an antibody from which the antigen-binding polypeptide is derived (e.g., Cys-226 or Cys-227 according to Kabat numbering). In some embodiments, the scaffold protein is selected from the group consisting of (a) apolipoprotein A, (b) apolipoprotein B, (c) apolipoprotein C, (d) apolipoprotein D, (e) apolipoprotein H, (f) apolipoprotein E, (g) a truncated version of (a)-(f) capable of stabilizing said bilayer, and (h) a combination of any one or more of (a)-(g); and/or the membrane-forming lipid is selected from the group consisting of C₄₋₂₈ fatty-acyl (e.g., a C₁₆ fatty-acyl), DMPC, DOPC, DOPS, DOPE, DPPC, and a combination of any one or more thereof. In some embodiments, one or more of said membrane-forming lipids is a C₄₋₂₈ fatty-acyl and the fatty-acyl is conjugated to a short peptide of 20-60 amino acids.

In some embodiments, provided is a conjugate comprising: a scaffold protein, a membrane-forming lipid, and a short peptide of 20-60 amino acids, wherein said membrane-forming lipids arrange in a lipid bilayer and said scaffold protein encircles said bilayer; and wherein said short peptide is conjugated to one or more of said membrane-forming lipids on one or both surfaces of said bilayer. In some embodiments, the membrane-forming lipids are selected from the group consisting of C₄₋₂₈ fatty-acyl (e.g., a C₁₆ fatty-acyl), DMPC, DOPC, DOPS, DOPE, DPPC, and a combination of any one or more thereof. In some embodiments, the membrane-forming lipid is a C₄₋₂₈ fatty-acyl and said fatty-acyl is conjugated to the short peptide of 20-60 amino acids.

In some embodiments, the fatty-acyl is a C₁₆ fatty-acyl. In some embodiments, the short peptide is a cystine-knot peptide (CKP). In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-II.

In some embodiments, the activity of the short peptide in the conjugate (e.g., in an NLP conjugate that comprises a short peptide and an antigen-binding polypeptide) is higher than the activity of the peptide in a conjugate that does not comprise the antigen-binding polypeptide. In some embodiments, the activity of the peptide in the conjugate is between 2-fold and 10-fold higher than the activity of the peptide in the conjugate that does not comprise the antigen-binding polypeptide. In some embodiments, the activity of the peptide in the conjugate is about 5-fold higher than the activity of the peptide in the conjugate that does not comprise the antigen-binding polypeptide. In some embodiments, the short peptide is a CKP or CKP variant having biological activity, and 80-90%, 90-95%, or 95-99% of said activity is retained in said conjugate. In some embodiments, said conjugate increases an activity or avidity of said short peptide. In some embodiments, the conjugate (e.g., the peptide-NLP conjugate) further comprises an antigen-binding polypeptide conjugated to one or more of said membrane-forming lipids on one or both surfaces of said bilayer via a functionalized group on said one or more membrane-forming lipids and a complementary functional group located C-terminally on said antigen-binding polypeptide; optionally wherein a spacer connects said functionalized group to said membrane-forming lipid and/or a spacer connects said complementary functional group to said antigen-binding polypeptide In some embodiments, the conjugate comprise 1-100 molecules of said Fab; optionally said conjugate comprises 5-30, 10-25, 15-20, or 18 molecules of said peptide, and 5-40, 10-35, 15-30, 20-25, or 23 molecules of said antigen binding polypeptide (e.g., Fab); or optionally said conjugate comprise 3-30, 5-20, 10-15, or 13 molecules of said peptide and 10-60 molecules of said antigen binding polypeptide (e.g., Fab); or optionally said conjugate comprise 20-80, 25-70, 30-60, 35-50, or 40 molecules of said peptide and 10-60 molecules of said antigen binding polypeptide (e.g., Fab). In some embodiments, the conjugate comprises at least two different peptide-C₄₋₂₈ fatty-acyl conjugates, wherein a first peptide-C₄₋₂₈ fatty-acyl conjugate comprises a first short peptide, and wherein a second peptide-C₄₋₂₈ fatty-acyl comprises a second short peptide that is different from the first short peptide. In some embodiments, the at least two peptides (e.g., cystine-knot peptides) bind different targets, optionally two, three, four, or eight different targets. In some embodiments, the two or more short peptides bind two different targets. In some embodiments, the targets are a pair selected from the group consisting of CD3 and CD19; CD3 and EpCAM; CD3 and CEA; CD16 and CD30; CD16 and CD33; Ang-2 and VEGF-A; and Factor X and Factor IXa.

In some embodiments, the scaffold protein and membrane-forming lipids are in a molar ratio of from 1:60 to 1:100, such as 1:80. In some embodiments, 20-35% of membrane-forming lipids carry the functionalized groups, such as 20%. The antigen-binding polypeptide may be selected from the group consisting of a Fab, a Fab′, a Fab′-SH, a F(ab′)₂, a single chain Fab (scFab), a single chain Fv (scFv), a VH-VH dimer, a VL-VL dimer, a VH-VL dimer, a single domain, a diabody, a linear antibody, and a combination of any one or more thereof. In particular embodiments, the antigen-binding polypeptide is a Fab or a Fab-like molecule, such as a human or humanized Fab. The antigen bound by the antigen-binding polypeptide (e.g., a Fab) may be OX40, DR4, GITR, Tie2, factor D, VEGF, MerTK, CD3, and/or Lymphotoxin beta receptor.

The conjugates can provide formats for multivalent and/or multi-specific constructs. For example, a conjugate may comprise two or more antigen-binding polypeptide molecules (e.g., two or more Fab molecules), such as 2-60, 2-32, 10-30, or 20 molecules. In some embodiments, the spacer comprises PEG or the amino acid sequence of GSGS. In some embodiments, the conjugate has 1-160, 10-120, 20-100, 40-80, or 60 molecules of said antigen-binding polypeptide; wherein said antigen-binding polypeptide is a Fab and said Fab is connected by a PEG spacer to said membrane-forming lipid. In some embodiments, the PEG spacer connects said functionalized group to said membrane-forming lipid and has a MW of 1000-3000, 1500-2500, 1900-2200, or 2000.

Such formats can increase activity and/or avidity of the antigen-binding polypeptide, its serum stability and/or its serum half-life (compared to the antigen-binding polypeptide not conjugated to the nanolipoprotein particle (NLP)). In some embodiments, the serum half-life of the antigen-binding polypeptide is increased by 2-20 fold, 5-15, or 10-fold. In some embodiments, said conjugate has 5-60, 6-40, or 7-32 molecules of said antigen-binding polypeptide.

In some embodiments, the conjugate comprises two or more antigen-binding polypeptides (e.g. two or more Fabs) binding different targets or epitopes, such as two, three, four, or eight different targets or epitopes. Such formats can provide bispecific constructs, e.g., where the targets are a pair selected from the group consisting of CD3 and CD19; CD3 and EpCAM; CD3 and CEA; CD16 and CD30; CD16 and CD33; Ang-2 and VEGF-A; and Factor X and Factor IXa. In some embodiments, the antigen-binding polypeptide is a Fab or a Fab-like molecule, optionally a human or humanized Fab.

Conjugates of the invention may be provided in pharmaceutical compositions comprising the conjugate and a pharmaceutically acceptable vehicle.

Another aspect of the invention provides high concentration, low viscosity liquid formulations, such as a liquid formulation comprising 100-300 mg of conjugate and having a viscosity of 10-50 cP. In some embodiments, the pharmaceutical composition, or liquid formulation, is administered subcutaneously or via ocular delivery. In some embodiments, the conjugate, composition, or formulation is lyophilized, such as in the presence of trehalose; and optionally reconstituted. In particular embodiments, lyophilization occurs in the presence of 80 mM trehalose; and/or at a concentration of 5 mg conjugate/mL. In some embodiments, the conjugate comprises 18-20 Fab molecules per conjugate. Advantageously, in some embodiments, 80-90%, 90-95%, or 95-99% of antigen-binding activity of the antigen-binding polypeptide (e.g., the Fab) is retained following lyophilization and reconstitution.

Another aspect of the invention provides methods of preparing a conjugate of the invention. In some embodiments, the method comprises a) providing a scaffold protein and a membrane-forming lipid under conditions allowing assembly of a nanolipoprotein particle comprising a lipid bilayer of said membrane-forming lipids encircled by said scaffold protein, where one or more membrane-forming lipids present a functionalized group on one or both surfaces of the particle; b) allowing the particle to contact an antigen-binding polypeptide (e.g., a Fab) having a C-terminally located complementary functional group that conjugates to the functionalized group at low pH in the range of 4.5 to 6.5 pH; and c) optionally purifying the conjugate of the particle and the antigen-binding polypeptide. In some embodiments, step b) follows step a) without an intervening step to remove some or all unassembled membrane-forming lipids. In particular embodiments, the functionalized group is a maleimide derivative and the functional group is a cysteine thiol group, such as where the cysteine forms a hinge disulfide bond in an antibody from which the antigen-binding polypeptide is derived (e.g., Cys-226 or Cys-227 according to Kabat numbering).

In some embodiments, the functionalized group does not conjugate to the scaffold protein at the low pH, e.g., at pHs of 5.5 to 6.5, 5-6, or a pH of 6.

In some embodiments of the methods, a spacer connects said functionalized group to said membrane-forming lipid and/or a spacer connects said complementary functional group to said antigen-binding polypeptide. In some embodiments, the spacer is a PEG spacer that connects said functionalized group to said membrane-forming lipid. In some embodiments, the PEG spacer has a MW of 1000-3000, 1500-2500, 1900-2200, or 2000.

In some embodiments, one or more of said membrane-forming lipids is a C₄₋₂₈ fatty-acyl and said fatty-acyl is conjugated to a short peptide of 20-60 amino acids. In some embodiments, the fatty-acyl is a C₁₆ fatty-acyl. In some embodiments, the short peptide is a cystine-knot peptide (CKP). In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-II. In some embodiments, the membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide. In some embodiments, the activity of the short peptide in the conjugate is higher than the activity of the peptide in a conjugate that does not comprise the antigen-binding polypeptide. In some embodiments, the activity of the peptide in the conjugate is between 2-fold and 10-fold higher than the activity of the peptide in the conjugate that does not comprise the antigen-binding polypeptide. In some embodiments, the activity of the peptide in the conjugate is about 5-fold higher than the activity of the peptide in the conjugate that does not comprise the antigen-binding polypeptide. In some embodiments, the short peptide is a CKP or CKP variant having biological activity, and 80-90%, 90-95%, or 95-99% of said activity is retained in said conjugate. In some embodiments, the conjugate comprise 1-100 molecules of said Fab; optionally said conjugate comprises 5-30, 10-25, 15-20, or 18 molecules of said peptide, and 5-40, 10-35, 15-30, 20-25, or 23 molecules of said antigen binding polypeptide (e.g., Fab); or optionally said conjugate comprise 3-30, 5-20, 10-15, or 13 molecules of said peptide and 10-60 molecules of said antigen binding polypeptide (e.g., Fab); or optionally said conjugate comprise 20-80, 25-70, 30-60, 35-50, or 40 molecules of said peptide and 10-60 molecules of said antigen binding polypeptide (e.g., Fab). In some embodiments, the conjugate comprises at least two different peptide-C₄₋₂₈ fatty-acyl conjugates, wherein a first peptide-C₄₋₂₈ fatty-acyl conjugate comprises a first short peptide, and wherein a second peptide-C₄₋₂₈ fatty-acyl comprises a second short peptide that is different from the first short peptide. In some embodiments, the at least two peptides (e.g., cystine-knot peptides) bind different targets, optionally two, three, four, or eight different targets. In some embodiments, the two or more short peptides bind two different targets. In some embodiments, the targets are a pair selected from the group consisting of CD3 and CD19; CD3 and EpCAM; CD3 and CEA; CD16 and CD30; CD16 and CD33; Ang-2 and VEGF-A; and Factor X and Factor IXa.

In some embodiments, provided is a method of preparing a conjugate of a nanolipoprotein particle and a short peptide of 20-60 amino acids (e.g., a cystine-knot peptide), comprising: a) providing a scaffold protein and a membrane-forming lipid under conditions allowing (self-)assembly of a nanolipoprotein particle comprising a lipid bilayer of said membrane-forming lipids encircled by said scaffold protein; wherein one or more of said membrane-forming lipids comprises a C₄₋₂₈ fatty-acyl (e.g., a C₁₆ fatty-acyl) and fatty-acyl is conjugated to said short peptide; and optionally purifying the peptide conjugate. In some embodiments, the short peptide is a cystine-knot peptide (CKP). In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-II. In some embodiments, said membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide.

In some embodiments, the conjugate comprises at least two different peptide-C₄₋₂₈ fatty-acyl conjugates, wherein a first peptide-C₄₋₂₈ fatty-acyl conjugate comprises a first short peptide, and wherein a second peptide-C₄₋₂₈ fatty-acyl comprises a second short peptide that is different from the first short peptide. In some embodiments, the least two peptides (e.g., cystine-knot peptides) bind different targets, optionally two, three, four, or eight different targets. In some embodiments, the two or more short peptides bind two different targets. In some embodiments, said targets are a pair selected from the group consisting of CD3 and CD19; CD3 and EpCAM; CD3 and CEA; CD16 and CD30; CD16 and CD33; Ang-2 and VEGF-A; and Factor X and Factor IXa. In some embodiments, short peptide is a CKP or CKP variant having biological activity, and 80-90%, 90-95%, or 95-99% of said activity is retained in said conjugate. In some embodiments, one or more of said membrane-forming lipids presents a functionalized group on one or both surfaces of said particle; and further comprising the step of: b) allowing said particle to contact an antigen-binding polypeptide (e.g., a Fab) having a C-terminally located complementary functional group that conjugates to said functionalized group at low pH in the range of 4.5 to 6.5 pH; wherein said step b) follows said step a) without an intervening step to remove some or all unassembled membrane-forming lipids; and/or without an intervening step to enrich the peptide conjugate of step a). In some embodiments, said functionalized group is a maleimide derivative and said functional group is a cysteine thiol group, optionally wherein said cysteine amino acid residue forms a hinge disulfide bond in the antibody from which the antigen-binding polypeptide is derived (such as Cys-226 or Cys-227). In some embodiments, said functionalized group does not conjugate to said scaffold protein at said low pH. In some embodiments, said low pH is a pH of 5.5 to 6.5, a pH of 5-6, or a pH of 6. In some embodiments, a spacer connects said functionalized group to said membrane-forming lipid and/or a spacer connects said complementary functional group to said antigen-binding polypeptide. In some embodiments, said spacer is a PEG spacer that connects said functionalized group to said membrane-forming lipid. In some embodiments, said PEG spacer has a MW of 1000-3000, 1500-2500, 1900-2200, or 2000. In some embodiments, said conjugate increases an activity or avidity of said short peptide. In some embodiments, said conjugate comprise 1-100 molecules of said Fab; optionally said conjugate comprises 5-30, 10-25, 15-20, or 18 molecules of said peptide, and 5-40, 10-35, 15-30, 20-25, or 23 molecules of said antigen binding polypeptide (e.g., Fab); or optionally said conjugate comprise 3-30, 5-20, 10-15, or 13 molecules of said peptide and 10-60 molecules of said antigen binding polypeptide (e.g., Fab); or optionally said conjugate comprise 20-80, 25-70, 30-60, 35-50, or 40 molecules of said peptide and 10-60 molecules of said antigen binding polypeptide (e.g., Fab). In some embodiments, the activity of the short peptide in the conjugate is higher than the activity of the peptide in a conjugate that does not comprise the antigen-binding polypeptide. In some embodiments, the activity of the peptide in the conjugate is between 2-fold and 10-fold higher than the activity of the peptide in the conjugate that does not comprise the antigen-binding polypeptide. In some embodiments, the activity of the peptide in the conjugate is about 5-fold higher than the activity of the peptide in the conjugate that does not comprise the antigen-binding polypeptide.

Also provided is a conjugate produce by any of the methods herein.

Accordingly, another aspect of the invention provides a method of reducing lipid-scaffold protein conjugation in preparing a conjugate of a nanolipoprotein particle and an antigen-binding polypeptide. The method may comprise a) providing a scaffold protein and a membrane-forming lipid under conditions allowing assembly of a nanolipoprotein particle comprising a lipid bilayer of membrane-forming lipids encircled by the scaffold protein, where one or more membrane-forming lipids presents a functionalized group on one or both surfaces of the particle; b) allowing the particle to contact an antigen-binding polypeptide (e.g., a Fab) having a C-terminally located complementary functional group at a low pH that favors conjugation of the functionalized group to the functional group rather than to the scaffold protein (e.g., a pH of about 5.55 to about 6.5, about 5 to about 6, or about 6). In some embodiments, step b) follows step a) without an intervening step to remove some or all unassembled membrane-forming lipids. In particular embodiments, the functionalized group is a maleimide derivative and the functional group is a cysteine thiol group, such as where the cysteine forms a hinge disulfide bond in an antibody from which the antigen-binding polypeptide is derived (e.g., Cys-226 or Cys-227 according to Kabat numbering). In some embodiments, said membrane-forming lipids is a C₄28 fatty-acyl and said fatty-acyl is conjugated to a short peptide of 20-60 amino acids. In some embodiments, said fatty-acyl is a C₁₆ fatty-acyl. In some embodiments, said short peptide is a cystine-knot peptide (CKP). In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, said membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide.

Another aspect of the invention provides methods of increasing avidity, activity, and/or potency of an antigen-binding polypeptide (e.g., a Fab) (compared to the polypeptide's avidity, activity, and/or potency when not conjugated to the NLP). In some embodiments, the method comprises a) providing a scaffold protein and a membrane-forming lipid under conditions allowing (self-)assembly of a nanolipoprotein particle comprising a lipid bilayer of said membrane-forming lipids encircled by said scaffold protein, wherein one or more of said membrane-forming lipids presents a functionalized group on one or both surfaces of said particle; b) allowing said particle to contact an antigen-binding polypeptide (e.g., a Fab) having a C-terminally located complementary functional group that conjugates to said functionalized group at low pH in the range of 4.5 to 6.5 pH; and c) optionally purifying the conjugate of said particle and said antigen-binding polypeptide, thereby increasing avidity, activity, and/or potency of the antigen-binding polypeptide (compared to the polypeptide's avidity, activity, and/or potency when not conjugated to the NLP). In particular embodiments, the antigen-binding polypeptide is a Fab or a Fab-like molecule; in particular embodiments, the conjugate comprises at least 6 molecules, e.g., 10-60, 15-30, or 20 molecules, of antigen-binding polypeptide. In some embodiments, said antigen-binding polypeptide is a Fab or a Fab-like molecule connected by a PEG spacer to said membrane-forming lipid; and said conjugate comprises 1-160, 10-120, 20-100, 40-80, or 60 molecules of said antigen-binding polypeptides. In some embodiments, the PEG spacer connects said functionalized group to said membrane-forming lipid and has a MW of 1000-3000, 1500-2500, 1900-2200, or 2000. In some embodiments, said conjugate is stabilized. In some embodiments, the conjugate is stabilized, such as conjugates comprising 7-60, 7-32, 40-60, or 50 molecules of said antigen-binding polypeptides, compared to conjugates comprising fewer or no antigen-binding polypeptides (e.g., fewer than 7 Fabs). In some embodiments, the conjugate further comprises one or more molecules of a second antigen-binding polypeptide that binds a different target; and/or the conjugate is provided in a liquid formulation comprising 100-300 mg of said conjugate and having a viscosity of 10-50 cP.

In some embodiments, one or more of said membrane-forming lipids is a C₄₋₂₈ fatty-acyl and said fatty-acyl is conjugated to a short peptide of 20-60 amino acids, and the method increases the avidity, activity, and/or potency of the short peptide. In some embodiments, said fatty-acyl is a C₁₆ fatty-acyl. In some embodiments, said short peptide is a cystine-knot peptide (CKP).

In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-JI. In some embodiments, said membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide. In some embodiments, the conjugate comprises at least two different peptide-C₄28 fatty-acyl conjugates, wherein a first peptide-C₄₋₂₈ fatty-acyl conjugate comprises a first short peptide, and wherein a second peptide-C₄₋₂₈ fatty-acyl comprises a second short peptide that is different from the first short peptide. In some embodiments, said at least two peptides (e.g., cystine-knot peptides) bind different targets, optionally two, three, four, or eight different targets. In some embodiments, said two or more short peptides bind two different targets. In some embodiments, said targets are a pair selected from the group consisting of CD3 and CD19; CD3 and EpCAM; CD3 and CEA; CD16 and CD30; CD16 and CD33; Ang-2 and VEGF-A; and Factor X and Factor IXa. In some embodiments, said conjugate comprise 1-100 molecules of said Fab; optionally said conjugate comprises 5-30, 10-25, 15-20, or 18 molecules of said peptide, and 5-40, 10-35, 15-30, 20-25, or 23 molecules of said antigen binding polypeptide (e.g., Fab); or optionally said conjugate comprise 3-30, 5-20, 10-15, or 13 molecules of said peptide and 10-60 molecules of said antigen binding polypeptide (e.g., Fab); or optionally said conjugate comprise 20-80, 25-70, 30-60, 35-50, or 40 molecules of said peptide and 10-60 molecules of said antigen binding polypeptide (e.g., Fab). In some embodiments, the activity of the short peptide in the conjugate is higher than the activity of the peptide in a conjugate that does not comprise the antigen-binding polypeptide. In some embodiments, the activity of the peptide in the conjugate is between 2-fold and 10-fold higher than the activity of the peptide in the conjugate that does not comprise the antigen-binding polypeptide. In some embodiments, the activity of the peptide in the conjugate is about 5-fold higher than the activity of the peptide in the conjugate that does not comprise the antigen-binding polypeptide.

Another aspect of the invention provides methods of increasing stability, shelf-life, and/or half-life of an antigen-binding polypeptide (e.g., a Fab) (compared to the polypeptide's avidity, activity, and/or potency when not conjugated to the NLP). Also provided are methods of increasing stability, shelf-life, and/or half-life of an NLP (compared to the NLP's increasing stability, shelf-life, and/or half-life when not conjugated to the antigen-binding polypeptide, e.g., a Fab or Fab-like molecule). In some embodiments, the method comprises a) providing a scaffold protein and a membrane-forming lipid under conditions allowing (self-)assembly of a nanolipoprotein particle comprising a lipid bilayer of said membrane-forming lipids encircled by said scaffold protein, wherein one or more of said membrane-forming lipids presents a functionalized group on one or both surfaces of said particle; b) allowing said particle to contact an antigen-binding-polypeptide (e.g., a Fab) having a C-terminally located complementary functional group that conjugates to said functionalized group at low pH in the range of 4.5 to 6.5 pH; and c) optionally purifying the conjugate of said particle and said antigen-binding polypeptide, thereby increasing stability, shelf-life, and/or half-life of the antigen-binding polypeptide (compared to the polypeptide's stability, shelf-life, and/or half-life when not conjugated to the NLP or conjugated to an NLP having fewer antigen-binding polypeptides, (e.g., fewer than 7 Fabs). In some embodiments, the conjugate comprises 5-60 molecules of said antigen-binding polypeptide. In particular embodiments, the antigen-binding polypeptide is a Fab or a Fab-like molecule; in particular embodiments, the conjugate comprises 7-32 molecules of antigen-binding polypeptide.

In some embodiments, one or more of the membrane-forming lipids is a C₄28 fatty-acyl and said fatty-acyl is conjugated to a short peptide of 20-60 amino acids. In some embodiments, said fatty-acyl is a C₁₆ fatty-acyl.

Also provided are methods of increasing the stability, shelf-life, and/or half-life of a short peptide that is 20-60 amino acids in length, said method comprising: a) providing a scaffold protein and a membrane-forming lipid under conditions allowing (self-)assembly of a nanolipoprotein particle comprising a lipid bilayer of said membrane-forming lipids encircled by said scaffold protein; one or more of said membrane-forming lipids comprises a C₄₋₂₈ fatty-acyl (e.g., a C₁₆ fatty-acyl) and fatty-acyl is conjugated to said short peptide; and optionally purifying the peptide conjugate; b) modifying one or more of said membrane-forming lipids to a present functionalized group on one or both surfaces of said particle, and c) allowing said particle to contact an antigen-binding polypeptide (e.g., a Fab) having a C-terminally located complementary functional group that conjugates to said functionalized group at low pH in the range of 4.5 to 6.5 pH; wherein said step c) follows said step b) without an intervening step to remove some or all unassembled membrane-forming lipids; and/or without an intervening step to enrich the peptide conjugate of step b). In some embodiments, a spacer connects said functionalized group to said membrane-forming lipid and/or a spacer connects said complementary functional group to said antigen-binding polypeptide, thereby increasing stability, shelf-life, and/or half-life of said short peptide. In some embodiments, the fatty-acyl is a C₁₆ fatty-acyl.

In some embodiments, said short peptide is a cystine-knot peptide (CKP). In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-II. In some embodiments, said membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide.

In some embodiments, said antigen-binding polypeptide is a Fab or a Fab-like molecule and/or said conjugate comprises 5-60, 6-40, 7-32, 40-60, or 50 molecules of said antigen-binding polypeptides (e.g., said Fab). In some embodiments, conjugate comprise 1-100 molecules of said Fab; optionally said conjugate comprises 5-30, 10-25, 15-20, or 18 molecules of said peptide, and 5-40, 10-35, 15-30, 20-25, or 23 molecules of said antigen-binding polypeptide (e.g., Fab); or optionally said conjugate comprise 3-30, 5-20, 10-15, or 13 molecules of said peptide and 10-60 molecules of said antigen binding polypeptide (e.g., Fab); or optionally said conjugate comprise 20-80, 25-70, 30-60, 35-50, or 40 molecules of said peptide and 10-60 molecules of said antigen binding polypeptide (e.g., Fab).

In some embodiments of the methods for increasing avidity, activity, potency, stability, shelf-life, and/or half-life, the conjugate further comprises a biologically active agent or a detectable agent. In particular embodiments, the biologically active agent is a cytotoxic agent selected from the group consisting of daunomycin, doxorubicin, methotrexate, vindesine, a radionuclide, diphtheria toxin, ricin, geldanamycin, a maytansinoid, calicheamicin, or a combination of any one or more thereof. In some embodiments, the detectable agent is a label selected from the group consisting a radioactive isotope, a fluorophore, a chemiluminescent dye, a chromophore, an enzyme, a metal ion, a nanoparticle, and a combination of any one or more thereof. In some embodiments, the conjugate is provided in a liquid formulation, said formulation comprising 100-300 mg of said conjugate and having a viscosity of 10-50 cP.

Other aspects of the invention provide a conjugate, pharmaceutical composition, or formulation as provided herein, for use as a medicament; for use in treating cancer or ocular disorders; in the manufacture of a medicament for treatment of cancer or ocular disorders; and/or in the manufacture of a medicament for inhibiting angiogenesis and/or tumor growth.

Another aspect of the invention provides a method of treating an individual with an antigen-binding polypeptide (e.g., a Fab) and/or a short peptide, comprising administering to the individual an effective amount of a conjugate, pharmaceutical composition, or formulation provided herein. In some embodiments, the method further comprises administering an additional therapeutic agent to the individual.

Still another aspect provides a method of delivering an antigen-binding polypeptide (e.g., a Fab) to an individual in need thereof, comprising providing a conjugate, pharmaceutical composition, or formulation of the invention, for administration to the individual. Advantageously, the antigen-binding polypeptide (e.g., a Fab) can be delivered in a potent, stable liquid formulation, such as where a conjugate comprises membrane-forming lipids conjugated to 5-60 molecules of antigen-binding polypeptide (e.g., 5-60 Fab molecules) via functionalized groups on the membrane-forming lipids and C-terminally located complementary functional groups on the antigen-binding polypeptide. In some embodiments, a spacer connects the functionalized group to the membrane-forming lipid and/or a spacer connects the complementary functional group to the antigen-binding polypeptide. Advantageously, the antigen-binding polypeptide (e.g., a Fab) can be delivered in a potent, low viscosity formulation, such as in a liquid formulation having a viscosity of 10-50 cP and a concentration of 100-300 mg of conjugate/mL.

In some embodiments of the methods for delivering antigen-binding polypeptides, the antigen-binding polypeptide is a Fab that binds a target selected from the group consisting of factor D, VEGF, Tie2, DR4, and a combination of any one or more thereof; and the formulation is administered via ocular delivery. In some embodiments of the methods for delivering antigen-binding polypeptides, the antigen-binding polypeptide is a Fab that binds a target selected from the group consisting of OX40, DR4, GITR, Tie2, factor D, VEGF, MerTK, CD3, Lymphotoxin beta receptor, and a combination of any one or more thereof. In particular embodiments, the antigen-binding polypeptides (e.g., the Fabs) bind at least two different targets, e.g., where the two different targets are a pair selected from the group consisting of CD3 and CD19; CD3 and EpCAM; CD3 and CEA; CD16 and CD30; CD16 and CD33; Ang-2 and VEGF-A; and Factor X and Factor IXa.

Also provided herein is a method of delivering a short peptide 20-60 amino acids in length to an individual in need thereof, in a potent, stable liquid formulation, said method comprising: administering to an individual a liquid formulation comprising a conjugate of said antigen-binding polypeptide (e.g., said Fab), and a nanolipoprotein particle; wherein said nanolipoprotein particle comprises a scaffold protein encircling a bilipid layer of membrane-forming lipids; wherein one or more of said membrane-forming lipids is a C₄₋₂₈ fatty-acyl and wherein said fatty-acyl is conjugated to a short peptide of 20-60 amino acids; wherein said membrane-forming lipids are conjugated to 5-100 molecules of said antigen-binding polypeptide via a functionalized group on said membrane-forming lipid and a C-terminally located complementary functional group on said antigen-binding polypeptide; optionally wherein a spacer connects said functionalized group to said membrane-forming lipid and/or a spacer connects said complementary functional group to said antigen-binding polypeptide, thereby delivering said antigen-binding polypeptide to an individual in a stable liquid formulation.

Also provided is a method of delivering a short peptide 20-60 amino acids in length to an individual in need thereof, in a potent, low viscosity formulation, said method comprising: administering to an individual a liquid formulation comprising a conjugate of said antigen-binding polypeptide (e.g., said Fab) and a nanolipoprotein particle; wherein said nanolipoprotein particle comprises a scaffold protein encircling a bilipid layer of membrane-forming lipids; wherein one or more of said membrane-forming lipids is a C₄₋₂₈ fatty-acyl and wherein said fatty-acyl is conjugated to a short peptide of 20-60 amino acids; wherein one or more of said membrane-forming lipids is conjugated to said antigen-binding polypeptide via a functionalized group on said one or more membrane-forming lipids and a C-terminally located complementary functional group on said antigen-binding polypeptide; optionally wherein a spacer connects said functionalized group to said membrane-forming lipid and/or a spacer connects said complementary functional group to said antigen-binding polypeptide; and wherein said liquid formulation has a viscosity of 10-50 cP and a concentration of 100-300 mg of said conjugate/mL, thereby delivering said antigen-binding polypeptide to an individual in a low viscosity formulation.

In some embodiments of the methods of delivering, said fatty-acyl is a C₁₆ fatty-acyl. In some embodiments, said short peptide is a cystine-knot peptide (CKP). In some embodiments, the CKP is EETI-II. In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-II. In some embodiments, said membrane-forming lipids comprise a peptide-C₄₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide. In some embodiments, the activity of the short peptide in the conjugate is higher than the activity of the peptide in a conjugate that does not comprise the antigen-binding polypeptide. In some embodiments, the activity of the peptide in the conjugate is between 2-fold and 10-fold higher than the activity of the peptide in the conjugate that does not comprise the antigen-binding polypeptide. In some embodiments, the activity of the peptide in the conjugate is about 5-fold higher than the activity of the peptide in the conjugate that does not comprise the antigen-binding polypeptide. In some embodiments, said conjugate comprise 1-100 molecules of said Fab; optionally said conjugate comprises 5-30, 10-25, 15-20, or 18 molecules of said peptide, and 5-40, 10-35, 15-30, 20-25, or 23 molecules of said antigen binding polypeptide (e.g., Fab); or optionally said conjugate comprise 3-30, 5-20, 10-15, or 13 molecules of said peptide and 10-60 molecules of said antigen binding polypeptide (e.g., Fab); or optionally said conjugate comprise 20-80, 25-70, 30-60, 35-50, or 40 molecules of said peptide and 10-60 molecules of said antigen binding polypeptide (e.g., Fab). In some embodiments, said spacer is a PEG spacer that connects said functionalized group to said membrane-forming lipid. In some embodiments, the PEG spacer has a MW of 1000-3000, 1500-2500, 1900-2200, or 2000. In some embodiments, said antigen-binding polypeptide (e.g., a Fab) and said short peptide each bind the same target. In some embodiments, said antigen-binding polypeptide (e.g., a Fab) and said cystine-knot peptide each bind a different target. In some embodiments, said antigen-binding polypeptide is a Fab that binds a target selected from the group consisting of factor D, VEGF, Tie2, DR4; and a combination of any one or more thereof; and said formulation is administered via ocular delivery. In some embodiments, said short peptide binds a target selected from the group consisting of factor D, VEGF, Tie2, DR4, and a combination of any one or more thereof. In some embodiments, said antigen-binding polypeptide is a Fab that binds a target selected from the group consisting of OX40, DR4, GITR, Tie2, factor D, VEGF, MerTK, CD3, Lymphotoxin beta receptor, and a combination of any one or more thereof. In some embodiments, said short peptide binds a target selected from the group consisting of OX40, DR4, GITR, Tie2, factor D, VEGF, MerTK, CD3, Lymphotoxin beta receptor, and a combination of any one or more thereof. In some embodiments, said antigen-binding polypeptides bind at least two different targets. In some embodiments, said two different targets are a pair selected from the group consisting of CD3 and CD19; CD3 and EpCAM; CD3 and CEA; CD16 and CD30; CD16 and CD33; Ang-2 and VEGF-A; and Factor X and Factor IXa. In some embodiments, said short peptide and said antigen-binding polypeptide bind at least two different targets. In some embodiments, said two different targets are a pair selected from the group consisting of CD3 and CD19; CD3 and EpCAM; CD3 and CEA; CD16 and CD30; CD16 and CD33; Ang-2 and VEGF-A; and Factor X and Factor IXa.

Yet still another aspect provides a method of delivering a biologically active agent or a detectable agent to an individual in need thereof, in a stable liquid formulation, the method comprising administering to an individual a liquid formulation comprising a conjugate of the invention and a biologically active agent or a detectable agent, where the conjugate comprises membrane-forming lipids conjugated to 5-60 molecules of antigen-binding polypeptide (e.g., 5-60 Fab molecules) via functionalized groups on the membrane-forming lipids and C-terminally located complementary functional groups on the antigen-binding polypeptide; and where the biologically active agent or detectable agent is conjugated to the scaffold protein, membrane-forming lipid and/or antigen-binding polypeptide of the conjugate. In some embodiments, a spacer connects the functionalized group to the membrane-forming lipid and/or a spacer connects the complementary functional group to the antigen-binding polypeptide.

Yet still another aspect provides a method of delivering a biologically active agent or a detectable agent to an individual in need thereof, in a low viscosity formulation, the method comprising administering to an individual a liquid formulation comprising a conjugate of the invention and a biologically active agent or a detectable agent, where the biologically active agent or detectable agent is conjugated to the scaffold protein, membrane-forming lipid, and/or antigen-binding polypeptide of the conjugate; and where the liquid formulation has a viscosity of 10-50 cP and a concentration of 100-300 mg conjugate/mL. In some embodiments, a spacer connects the functionalized group to the membrane-forming lipid and/or a spacer connects the complementary functional group to the antigen-binding polypeptide.

Yet still another aspect provides a method of delivering a biologically active agent or a detectable agent to the brain or central nervous system of an individual in need thereof, the method comprising systemically administering to an individual a liquid formulation comprising a conjugate of the invention and a biologically active agent or a detectable agent, where the biologically active agent or detectable agent is conjugated to the scaffold protein, membrane-forming lipid, and/or antigen-binding polypeptide; and where the conjugate translocates across the blood-brain barrier. In some embodiments, a spacer connects the functionalized group to the membrane-forming lipid and/or a spacer connects the complementary functional group to the antigen-binding polypeptide. In particular embodiments, the scaffold protein is apoE2 and/or apoE4. In particular embodiments, the liquid formulation is administered intravenously.

In some embodiments, one or more of said membrane-forming lipids is a C₄₋₂₈ fatty-acyl and said fatty-acyl is conjugated to a short peptide of 20-60 amino acids. In some embodiments, said fatty-acyl is a C₁₆ fatty-acyl. In some embodiments, said short peptide is a cystine-knot peptide (CKP). In some embodiments, the CKP is EETI-II In some embodiments, the short peptide is CKP variant that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the CKP variant comprises an additional lysine residue at its N-terminus relative to a wild type CKP. In some embodiments, the wild type CKP is EETI-II. In some embodiments, said membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9; and/or said conjugate comprises 1-100, 10-90, 20-80, 30-70, 40-60, or 60 molecules of said short peptide. In some embodiments, said spacer is a PEG spacer that connects said functionalized group to said membrane-forming lipid. In some embodiments, said PEG spacer has a MW of 1000-3000, 1500-2500, 1900-2200, or 2000.

In some embodiments of the methods for delivering a biologically active agent or a detectable agent, the antigen-binding polypeptide is a Fab or a Fab-like molecule. In particular embodiments, the biologically active agent is a cytotoxic agent selected from the group consisting of daunomycin, doxorubicin, methotrexate, vindesine, a radionuclide, diphtheria toxin, ricin, geldanamycin, a maytansinoid, calicheamicin, or a combination of any one or more thereof. In some embodiments, the detectable agent is a label selected from the group consisting a radioactive isotope, a fluorophore, a chemiluminescent dye, a chromophore, an enzyme, a metal ion, a nanoparticle, and a combination of any one or more thereof. In particular embodiments, the membrane-forming lipids are conjugated to 5-60 molecules of antigen-binding polypeptide. Advantageously, the liquid formulation may have a viscosity of 10-50 cP and a concentration of 100-300 mg of conjugate/mL.

In some embodiments, the liquid formulation is administered intravenously. In some embodiments, said membrane-forming lipids are conjugated to 5-100 molecules of said antigen-binding polypeptide via a functionalized group on said membrane-forming lipid and a C-terminally located complementary functional group on said antigen-binding polypeptide. In some embodiments, said antigen-binding polypeptide is a Fab and said Fab is connected via a PEG spacer to said membrane-forming lipid. In some embodiments, said PEG spacer connects said functionalized group to said membrane-forming lipid and has a MW of 1000-3000, 1500-2500, 1900-2200, or 2000. In some embodiments, said liquid formulation has a viscosity of 10-50 cP and a concentration of 100-300 mg of said conjugate/mL.

Another aspect provides a system or kit for carrying out the methods described herein, e.g., for delivering an antigen-binding polypeptide (e.g., a Fab) to a target cell. In some embodiments, the system or kit comprises one or more components for preparing a NLP-polypeptide conjugate or peptide-NLP-polypeptide conjugate herein, a pharmaceutical composition herein, or a formulation herein, in one or more compartments of the system or kit. In some embodiments, instructions are provided for preparing the conjugate, composition, or formulation, or for its use in delivering the antigen-binding polypeptide in accordance with methods of the invention. In particular embodiments, the antigen-binding protein is a Fab or a Fab-like molecule.

Also provided is a system or kit for delivering a short peptide of 20-60 amino acids (e.g. a cystine-knot peptide) to a target cell, the system or kit comprising: one or more components for preparing a peptide-NLP conjugate herein, a pharmaceutical composition herein, or a formulation herein, wherein said components are provided in one or more compartments of said kit or system, and optionally instructions for preparing said conjugate or composition and/or instructions for delivering said short peptide (e.g., said cystine-knot peptide).

In a related aspect, provided is a method of increasing the biological activity of a short peptide attached to the surface of a lipid-based nanoparticle, comprising (a) providing a lipid-based nanoparticle comprising a short peptide attached its surface, wherein one or more lipids of the lipid-based nanoparticle presents a functionalized group, and (b) allowing the nanoparticle to contact a polypeptide having a functional group under conditions that favor conjugation of said functionalized group to said functional group. In some embodiments, the method further comprises purifying the conjugate comprising the lipid based nanoparticle, the short peptide, and the polypeptide. In some embodiments, the lipid-based nanoparticle comprises a lipid bilayer. In some embodiments, the lipid-based nanoparticle is a liposome, a solid lipid nanoparticle (SLN), or a nanostructured lipid carrier (NLC). In some embodiments, a spacer connects said functionalized group to a lipid on the surface of the lipid-based nanoparticle and/or a spacer connects said complementary functional group to said polypeptide. In some embodiments, the polypeptide is an antigen-binding polypeptide. In some embodiments, the antigen-binding polypeptide is selected from the group consisting of a Fab, a Fab′, a Fab′-SH, a F(ab′)₂, a single chain Fab (scFab), a single chain Fv (scFv), a VH-VH dimer, a VL-VL dimer, a VH-VL dimer, a single domain, a diabody, a linear antibody, and a combination of any one or more thereof. In some embodiments, the short peptide is a CKP or a variant of a CKP that comprises: (a) one or more amino acid insertions, deletions, and/or substitutions in one or more loop sequences relative to the corresponding one or more loop sequences of a wild type CKP; (b) one or more amino acid insertions, deletions, and/or substitutions at the N terminus relative to a wild type CKP; (c) one or more amino acid insertions, deletions, and/or substitutions at the C-terminus relative to a wild type CKP; (d) a chemical modification at the N-terminus relative to a wild type CKP; and/or (e) a chemical modification at the C-terminus relative to a wild type CKP. In some embodiments, the activity of the short peptide following conjugation of the polypeptide is between 2-fold and 100-fold higher following conjugation of the polypeptide to the lipid-based nanoparticle as compared to the activity of the short peptide before conjugation of the polypeptide to the lipid-based nanoparticle.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D depict the effect of DOPE-MCC on NLP assembly. FIG. 1A depicts a schematic of DOPE-MCC NLP assembly and conjugation of a Fab. FIG. 1B depicts MALS/QELS analysis of MW and Rₕ across the NLP SEC peaks as a function of mol % DOPE-MCC in the NLP. FIG. 1C depicts TIC chromatograms of apoE422k alone (circles), NLP assembled at pH 7.4 (plain line) and NLP assembled at pH 6.0 (triangles). FIG. 1D depicts deconvoluted masses from the peak TIC spectra shown in 1D for apoE422k alone (circles), NLP assembled at pH 7.4 (plain line) and NLP assembled at pH 6.0 (triangles).

FIGS. 2A-2C depict Fab-NLP conjugation, purification and characterization. FIG. 2A depicts SEC purification of the Fab-NLP after conjugation at a Fab to NLP ratio of 20. The first peak corresponds to the Fab-NLP conjugate, the second to Fab dimer and the third to free unconjugated Fab. The inset shows a zoomed-in version of the Fab-NLP peak with Rₕ from QELS overlaid across the peak. The left y-axis is absorbance and the right y-axis is the Rₕ of different fractions of the Fab-NLP conjugate across the peak. FIG. 2B depicts TIC chromatograms of free Fab (circles) and Fab-NLP conjugate (triangles). FIG. 2C depicts deconvoluted masses from the peak TIC spectra shown in FIG. 2C for free Fab (circles) and the Fab-NLP conjugate (triangles).

FIGS. 3A-3D depict optimizing Fab loading. FIG. 3A depicts SEC purification chromatograms of Fab-NLP conjugates assembled at increasing Fab concentrations. FIG. 3B depicts the number of Fabs per NLP (Fab:NLP) as a function of Fab concentration in the conjugation reaction. FIG. 3C depicts MW of the Fab-NLP conjugate as a function of the number of Fabs per NLP (Fab:NLP). FIG. 3D depicts Rₕ of the Fab-NLP conjugate as a function of the number of Fabs per NLP (Fab:NLP).

FIGS. 4A-4B depict Fab-NLP formulation. FIG. 4A depicts the relationship between viscosity and Fab concentration for free Fab alone (circles), Fab-NLP conjugates (squares) and Fab-PEG conjugates (triangles). FIG. 4B depicts Rₕ and MW analysis of Fab-NLP conjugates before and after lyophilization. The left y-axis and gray bars are the MW and the right y-axis and black bars are the Rₕ.

FIGS. 5A-5D depict Fab-NLP activity. FIG. 5A depicts activity of anti-factor D Fab alone (crosses), conjugated to the NLP (Fab-NLP, squares), conjugated to NLP after lyophilization and reconstitution (triangles), and a negative Fab control (circles) in factor D FRET assay of factor B cleavage. Mean and standard deviation of quadruplicates are shown, data representative of n=3 independent experiments. FIG. 5B depicts agonist activity of the anti-OX40 when conjugated to the NLP platform at Fab valencies ranging from 0-29.4 plotted as a function of anti-OX40 fab concentration. Anti-OX40 huIgG1 was used as the negative control. Mean and standard deviations of triplicates are shown. FIG. 5C depicts agonist activity of the anti-OX40 when conjugated to the NLP platform at Fab valencies ranging from 0-29.4 plotted as a function of NLP concentration. Anti-OX40 huIgG1 was used as the negative control. Mean and standard deviations of triplicates are shown. FIG. 5D depicts EC50 values calculated from curve fits in FIG. 5C at the different Fab-NLP valencies.

FIGS. 6A-6B depict stability of Fab-NLP conjugate in 50% serum. FIG. 6A depicts SEC traces of NLP at various time points after incubation in 50% serum. The shoulder on the left of the main NLP peak was due to background absorbance in the serum and was subtracted in subsequent analyses. FIG. 6B depicts Fab-NLP degradation as a function of time for Fab-NLP conjugates consisting of different Fab loading densities. These curves were generated by normalizing to the Fab-NLP peak area at time 0 hr.

FIGS. 7A-7B depict quantifying the protein composition of Fab-NLP conjugates. FIG. 7A depicts HPLC trace of a Fab-NLP conjugate. Two peaks were observed at 4.95 min and 5.15 min that correspond to apoE422k and Fab, respectively. FIG. 7B depicts HPLC traces of apoE422k at different injection amounts (left panel) and the corresponding standard curve (right panel). The apoE422k standard curve was used to determine the apoE422k concentration of the Fab-NLP conjugate.

FIGS. 8A-8B depict effect of DOPE-MCC on NLP assembly. FIG. 8A depicts SEC analysis of NLPs assembled with increasing molar concentrations of DOPE-MCC. FIG. 8B depicts MW and Rₕ analysis of NLP assembled under crosslinking conditions (pH 7.4) and non-crosslinking conditions (pH 6).

FIGS. 9A-9D depict the design and production of C16-EETI-II. FIG. 9A shows the amino acids sequences of wild-type EETI-II and C16-EETI-II. Palmitic acid was conjugated through an N-terminal lysine onto EETI-II. FIG. 9B provides a flowchart of the process of C16-EETI-II production. FIG. 9C provides an analytical RP-HPLC (left panel) and LC-MS (right panel) of EETI-JI. LC-MS verifies the identity and purity of C18-EETI-II and EETI-II. FIG. 9D provides an analytical RP-HPLC (left panel) and LC-MS (right panel) that shows that C16-EETI-II is more hydrophobic than EETI-JI (indicated by the longer retention time of C16-EETI-II) with the addition of the fatty acid tag.

FIGS. 10A-10E depict assembly of CKP loaded NLPs (NLP-CKP). FIG. 10A depicts a schematic of CKP-NLP assembly and conjugation of a CKP. FIG. 10B depicts SEC chromatogram of CKP-NLP (dotted lines show the fraction that were pooled for further analysis). FIG. 10C depicts RP-HPLC chromatogram of the CKP-NLP using an ELSD (evaporating light scattering detector). Three peaks were observed corresponding to ApoE422k, CKP and DOPC as indicated by the respective cartoon renditions of each. FIG. 10D depicts SEC-MALS analysis of MW (MW is indicated with an arrow). The absorbance at 280 nm is shown on the right axis. FIG. 10E depicts SEC-MALS analysis of Rh (Rh (left axis) is indicated by scattered dots across the CKP-NLP peak). The absorbance at 280 nm is shown on the right axis.

FIGS. 11A-11D depict effects of CKP loading on NLP size and composition, as well as on CKP activity. FIG. 11A depicts SEC chromatogram of CKP-NLPs assembled at increasing CKP-C16 mol %. FIG. 11B depicts average Rh analysis across the CKP-NLP SEC peak as a function of CKP mol % in the CKP-NLP assembly. FIG. 11C depicts HPLC quantification of number of CKP molecules per NLP after purification as a function of the number of CKP molecules per NLP that was included in the self-assembly reaction. Assemblies were analyzed in duplicate FIG. 11D shows the results of a CKP activity assay for CKP-NLPs containing different levels of CKP incorporation (0-63 CKP/NLP). Assays were performed in triplicate. FIGS. 12A-12B depict SEC chromatograms of NLPs at different times after storage at 37° C. in 50% serum. FIG. 12A depicts results where NLPs were labeled with AF488 and the absorbance in the SEC trace was monitored at A495 to limit background signal from the biological matrix. FIG. 12B depicts normalized peak areas of CKP-NLPs as a function of time when incubated at 37° C. in 50% serum.

FIGS. 13A-13D depict assembly and characterization of Fab-CKP-NLP conjugates. FIG. 13A depicts a schematic of the strategy for generating Fab-CKP-NLP conjugates. The CKP-NLPS are assembled with a reactive DSPE-PEG-Mal lipid and the assembled CKP-NLP are conjugated to Fab via a free cysteine. FIG. 13B depicts a SEC chromatogram of the Fab-CKP-NLP conjugate after conjugation is complete. Three peaks are observed corresponding to the Fab-CKP-NLP conjugate, Fab dimer, and unconjugated Fab. FIG. 13C depicts HPLC chromatogram of the SEC purified Fab-CKP-NLP conjugate. All components of the Fab-CKP-NLP were detected as indicated by the respective cartoon rendition of each. FIG. 13D depicts SEC-MALS analysis of the MW (top panel) and Rh (bottom panel) across the Fab-CKP-NLP conjugate peak.

FIGS. 14A-14E depict Fab loading and stability of Fab-CKP-NLPs. FIG. 14A depicts SEC chromatograms of Fab-CKP-NLP conjugates generated at different Fab to CKP-NLP ratios (0-150) during the conjugation step. Three peaks are observed corresponding to the Fab-CKP-NLP conjugate, Fab dimer, and unconjugated Fab. FIG. 14B depicts HPLC analysis of the number of Fabs per NLP in the purified Fab-CKP-NLP conjugate as a function of the Fab ratio in the reaction for both Low CKP-NLP and High CKP-NLP. FIG. 14C depicts the Rh of the Fab-CKP-NLP as a function of Fab loading for both Low CKP-NLP and High CKP-NLP. FIG. 14D depicts the results of CKP activity assay for the Fab-CKP-NLP as a function of Fab loading for both Low CKP-NLP and High CKP-NLP. Assays were performed in triplicate. FIG. 14E depicts normalized peak areas of CKP-NLP and Fab-CKP-NLP as a function of time when incubated at 37° C. in 50% serum.

### DETAILED DESCRIPTION

### I. Definitions

Terms are used herein as generally used in the art, unless otherwise defined in the following.

The term “antibody” herein is used in the broadest sense and encompasses various antibody structures, including but not limited to monoclonal antibodies, polyclonal antibodies, and antibody fragments so long as they exhibit the desired antigen-binding activity. Accordingly, in context of the present invention, the term antibody relates to full immunoglobulin molecules as well as to parts of such immunoglobulin molecules. Furthermore, the term relates to modified and/or altered antibody molecules, in particular to mutated antibody molecules. The term also relates to recombinantly or synthetically generated/synthesized antibodies. In the context of the present invention the term antibody is used interchangeably with the term immunoglobulin.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and single-domain antibodies (e.g., nanobodies, IgNARs). For a review of certain antibody fragments, see Hudson et al., Nat Med 9, 129-134 (2003). For a review of scFv fragments, see e.g., Plückthun, in The Pharmacology of Monoclonal Antibodies, vol. 113, Rosenburg and Moore eds., Springer-Verlag, New York, pp. 269-315 (1994); see also WO 93/16185. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody (Domantis, Inc., Waltham, MA; see e.g., U.S. Pat. No. 6,248,516 B1). Further examples of antibody formats comprising various antibody fragments include, but are not limited to, BiTE, BiTE-Fc, DART, DART-Fc, TriKE, and TandAb formats (for a review, please see Suurs, F. V., et al., A review of bispecific antibodies and antibody constructs in oncology and clinical challenges, Pharmacology & Therapeutics, 201 (2019) 103-119). Antibody fragments can be made by various techniques, including but not limited to proteolytic digestion of an intact antibody as well as production by recombinant host cells (e.g., E. coli or phage).

As used herein, the term “antigen-binding polypeptide” refers in its broadest sense to a molecule that specifically binds an antigenic determinant. Examples of antigen-binding polypeptides are antibodies, immunoglobulins, and derivatives thereof, e.g., antibody fragments. Antigen-binding polypeptides may also include darpins and/or protein binders comprising a protein scaffold, such as for example, Centyrins, Affimers, AdNectins, Avimers Knottins Monobodies Affinity clamps, and the like. An antigen-binding polypeptide binds (i.e., specifically binds) to an antigenic determinant. In some embodiments, an antigen-binding polypeptide is able to direct the entity to which it is attached (e.g., a nanolipoprotein particle) to a target site, for example to a specific type of tumor cell or tumor stroma bearing the antigenic determinant. In some embodiments, an antigen-binding polypeptide is able to activate signaling through its target antigen, for example signaling is activated upon binding of an antigenic determinant to an antigen-binding receptor on a T cell. Antigen-binding polypeptides may include an antigen-binding domain, typically comprising an immunoglobulin heavy chain variable region and an immunoglobulin light chain variable region. In certain embodiments, the antigen-binding polypeptide may comprise immunoglobulin constant regions as known in the art. Useful heavy chain constant regions include any of the five isotypes: α, δ, ε, γ, or μ. Useful light chain constant regions include any of the two isotypes: κ and λ. In certain embodiments, the antigen-binding polypeptide does not comprise an Fc region. In certain embodiments, the antigen-binding polypeptide comprises a hinge region (or part of the hinge region of an immunoglobulin), but not an Fc region, or not all of the Fc region. For example, in some embodiments, the antigen-binding polypeptide does not include one or more subunits of an IgG Fc domain, e.g., not comprising an IgG CH2 constant domain; and/or not comprising an IgG CH3 constant domain.

As used herein, term “polypeptide” refers to a molecule composed of monomers (amino acids) linearly linked by amide bonds (also known as peptide bonds). The term polypeptide refers to any chain of two or more amino acids, and does not refer to a specific length of the product. Thus, peptides, dipeptides, tripeptides, oligopeptides, protein, amino acid chain, or any other term used to refer to a chain of two or more amino acids, are included within the definition of polypeptide, and the term polypeptide may be used instead of, or interchangeably with any of these terms. The term polypeptide is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of 3, 5, 10, 20, 25, 50, 75, 100, 200, 500, 1,000, or 2,000 amino acids.

An “antigen-binding site” refers to the site, i.e. one or more amino acid residues, of an antigen-binding polypeptide which provides interaction with the antigen. For example, the antigen-binding site of an antibody or an antigen-binding receptor comprises amino acid residues from the complementarity determining regions (CDRs). A native immunoglobulin molecule typically has two antigen-binding sites, a Fab or a scFv molecule typically has a single antigen-binding site.

The term “antigen-binding domain” refers to the part of an antibody or an antigen-binding receptor that comprises the area that specifically binds to, and is complementary to part or all of, an antigen. An antigen-binding domain may be provided by, for example, one or more immunoglobulin variable domains (also called variable regions). Typically, an antigen-binding domain comprises an immunoglobulin light chain variable region (VL) and an immunoglobulin heavy chain variable region (VH).

The term “variable region” or “variable domain” refers to the domain of an immunoglobulin heavy or light chain that is involved in binding the antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co, page 91 (2007). A single VH or VL domain is usually sufficient to confer antigen-binding specificity.

The term “binding to” as used in the context of antigen-binding polypeptides defines a binding (interaction) of an “antigen-interaction site” and an antigen with each other. The term “antigen-interaction site” defines a motif of a polypeptide which shows the capacity of specific interaction with a specific antigen or a specific group of antigens. Said binding/interaction is also understood to define a “specific recognition”. The term “specifically recognizing” means that the antigen-binding polypeptide is capable of specifically interacting with and/or binding to an antigen. The ability of the antigen-binding polypeptide (e.g. a Fab or scFv domain) to bind to a specific target antigenic determinant can be measured for techniques known in the art. One technique involves enzyme-linked immunosorbent assay (ELISA). Other techniques familiar to one of skill in the art include, e.g., surface plasmon resonance (SPR) (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen-binding polypeptide to an unrelated protein/antigen is less than 10% of the binding of the antigen-binding polypeptide to the target antigen as measured, in particular by SPR. In certain embodiments, an antigen-binding polypeptide that binds to the target antigen, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). The term “binding” or “specific binding,” as used in connection with an antigen-binding polypeptide, means that the antigen-binding polypeptide does not, or essentially does not cross-react structures that that the target antigen.

“Affinity” refers to the strength of the sum total of non-covalent interactions between a single binding site of a molecule (e.g., an antigen-binding polypeptide) and its binding partner (e.g., the antigen). Unless indicated otherwise, as used herein, “binding affinity” refers to intrinsic binding affinity, which reflects a 1:1 interaction between members of a binding pair (e.g., an antigen-binding polypeptide and an antigen, and/or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A preferred method for measuring affinity is Surface Plasmon Resonance (SPR) and a preferred temperature for the measurement is 25° C.

The term “CDR” as employed herein relates to “complementary determining region”, which is well known in the art. The CDRs are parts of immunoglobulins or antigen-binding receptors that determine the specificity of said molecules and make contact with a specific ligand. The CDRs are typically the most variable part of the molecule and contribute to the antigen-binding diversity of these molecules. There are three CDR regions CDR1, CDR2 and CDR3 in a variable domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in “Kabat” (Sequences of Proteins of Immunological Interest”, 5th edit. NIH Publication no. 91-3242 U.S. Department of Health and Human Services (1991); Chothia J. Mol. Biol. 196 (1987), 901-917) or “Chothia” (Nature 342 (1989), 877-883).

The term “hypervariable region” or “HVR”, as used herein, refers to each of the regions of an antibody variable domain that are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops. With the exception of CDR1 in VH, CDRs generally comprise the amino acid residues that form the hypervariable loops. Hypervariable regions (HVRs) are also referred to as complementarity determining regions (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. The appropriate amino acid residues which encompass the CDRs as defined by the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of Kabat numbering to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antigen-binding moiety variable region are according to the Kabat numbering system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “Fab” or a “Fab molecule” refers to a protein consisting of the VH and CH1 domain of the heavy chain (the “Fab heavy chain”) and the VL and CL domain of the light chain (the “Fab light chain”) of an antigen-binding polypeptide.

A single chain Fv fragment (or scFv) is a polypeptide consisting of an antibody VH domain and a VL domain connected by a peptide linker.

A single chain Fab fragment (or scFab) is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Single chain Fab fragments are stabilized via the natural disulfide bond between the CL domain and the CH1 domain.

By a “crossover Fab molecule” (also termed “crossFab” or “crossover Fab fragment”) is meant a Fab molecule wherein either the variable regions or the constant regions of the Fab heavy and light chain are exchanged, i.e. the crossFab fragment comprises a peptide chain composed of the light chain variable region and the heavy chain constant region, and a peptide chain composed of the heavy chain variable region and the light chain constant region. Accordingly, a crossFab fragment comprises a heavy or light chain composed of the heavy chain variable and the light chain constant regions (VH-CL), and a heavy or light chain composed of the light chain variable and the heavy chain constant regions (VL-CH1). In contrast thereto, by a “conventional Fab” molecule is meant a Fab molecule in its natural format, i.e. comprising a heavy chain composed of the heavy chain variable and constant regions (VH-CH1), and a light chain composed of the light chain variable and constant regions (VL-CL).

A “crossover single chain Fab fragment” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; and where the VH and VL form together an antigen-binding site which binds specifically to an antigen. Typically, the linker is a polypeptide of at least 30 amino acids, such as 30-40 amino acids in length.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fe region is usually defined to extend from Cys-226, or from Pro-230, to the carboxyl-terminus of the heavy chain. However, the C-terminal lysine (Lys-447) of the Fc region may or may not be present. Unless otherwise specified herein, numbering of amino acid residues in the Fc region or constant region is according to the “EU numbering” system, also called the EU index, as described in Kabat et al., Sequences of Proteins of Immunological Interest, 5th Ed. Public Health Service, National Institutes of Health, Bethesda, M D, 1991.

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG1 and IgG2), IgM, IgA, IgD, and IgE.) The full length antibodies used according to the invention can be from a single species e.g. human, or they can be chimerized or humanized antibodies.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human, and may be a clinical patient/clinical trial volunteer.

In accordance with this invention, the term “pharmaceutical composition” refers to a preparation in such form as to permit the biological activity of an active ingredient contained therein to be effective, and which contains no additional components which are unacceptably toxic to a subject to which the formulation would be administered.

By an “isolated polypeptide,” or a fragment or derivative thereof, refers to a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

As used herein, the term “cystine-knot peptide” or “CKP” refers to a peptide between 26-60 amino acids in length, which contain six conserved cysteine residues that form three disulfide bonds. One of the disulfides penetrates the macrocycle formed by the two other disulfides and their interconnecting backbones, thereby yielding a characteristic knotted topology with multiple loops exposed on the surface. The loops are defined as the amino acid regions which flank the six conserved cysteine residues and are highly variable in nature.

As used herein, the term “target antigen” is synonymous with “target antigenic determinant,” “target epitope” and “target cell antigen” and refers to a site (e.g., a contiguous stretch of amino acids or a conformational configuration made up of different regions of non-contiguous amino acids) on a polypeptide macromolecule to which an antigen-binding polypeptide binds, forming a complex. Useful target antigens can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g., CD20, CEA, FAP, TNC) can be any native form of the proteins from any vertebrate source, including mammals such as primates (e.g., humans) and rodents (e.g., mice and rats), unless otherwise indicated. In a particular embodiments, the target antigen is a human protein. Where reference is made to a specific target protein herein, the term encompasses the “full-length”, unprocessed target protein as well as any form of the target protein that results from processing in the target cell. The term also encompasses naturally occurring variants of the target protein, e.g., splice variants or allelic variants. Exemplary human target antigens include, but are not limited to: CD20, CEA, FAP, TNC, MSLN, FolR1, HER1 and HER2.

The terms “cancer” and “cancerous” refer to the physiological condition in mammals that is typically characterized by unregulated cell growth/proliferation. Included in this definition are benign and malignant cancers. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastric or stomach cancer including gastrointestinal cancer, pancreatic cancer, glioblastoma, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer (e.g., renal cell carcinoma), liver cancer, prostate cancer, vulval cancer, thyroid cancer, hepatic carcinoma, anal carcinoma, penile carcinoma, melanoma, and various types of head and neck cancer. By “early stage cancer” is meant a cancer that is not invasive or metastatic or is classified as a Stage 0, 1, or II cancer. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a nonmetastatic cancer is any cancer that is a Stage 0, I, or II cancer, and occasionally a Stage III cancer.

Numeric values provided herein are understood to refer to both the exact numeric value, as well as the value qualified by the term “about.” The term “about” qualifies the numeric value by a range as would be understood by one of skill in the art, in the context being used, e.g., by ±10%, ±5%, ±4%, ±3%, ±2%, ±1%, ±0.5%, ±0.1%, ±0.05%, 0.01%, and so forth, of the recited numeric value.

### II. Nanolipoprotein Conjugates Comprising a Polypeptide

One aspect of the invention relates to nanolipoprotein particle conjugates comprising at least one covalently conjugated polypeptide (that is, an NLP-incorporated polypeptide other than a scaffold protein). As used herein, “nanolipoprotein particles” or NLPs refer to supramolecular complexes comprising lipid and scaffold proteins, in particular, comprising membrane-forming lipids and scaffold proteins, such as apolipoproteins. NLPs also can be referred to as nanodiscs or reconstituted high density lipoproteins (rHDLs), and have been described as mimetics of endogenous high-density lipoproteins (HDLs) (18). NLPs generally are formed through a self-assembly process, where the membrane-forming lipids form a lipid bilayer having a discoidal shape (19), and where the hydrophobic periphery of the disc is stabilized by binding to the scaffold protein, which encircles the bilayer disc. All of the references cited in this paragraph can be found below Example 5.

A “membrane-forming lipid” refers to an amphipathic lipid or polar lipid, having a hydrophilic region (polar head) and a hydrophobic region (one or more long hydrocarbon tails). Membrane-forming lipid self-assembly in an aqueous environment to form a lipid bilayer, with the hydrophobic fatty acid tails of each layer facing each other, and the polar heads exposed to the aqueous environment. The polar head group is typically a derivatized phosphate or a saccharide group. The membrane-forming lipid may comprise, e.g., an allylphosphocholine, ether lipid, glycolipid, lysosphingolipid, lysoglycerophospholipids, phospholipid, sphingolipid, and sterol. In some embodiments, the NLP comprises two or more membrane forming lipids described herein. More specific examples include, but are not limited to, the phospholipids dimyristoylphosphatidylcholine (DMPC), dioleoylphospho-ethanolamine (DOPE), dioleoylphosphatidylcholine (DOPC), dioleoylphosphoserine (DOPS), and dipalmitoyl-phosphatidyl-choline (DPPC). In a particular embodiment, the membrane-forming lipid is DOPE and/or DOPC. In some embodiments, the membrane-forming lipids are non-lipid amphipathic molecules, such as digylcerol tetraethers, cholesterol, ergosterol, and the like. In some embodiments, the NLP comprises (such as further comprises) C₄₋₂₈ fatty-acyls (e.g., a C₁₆ fatty-acyl) as membrane-forming lipids.

In some embodiments, the membrane-forming lipid is a biological molecule, i.e. a molecule produced by a living organism, e.g., bacteria, yeast, or a mammal.

In NLPs, the bilayer formed by membrane-forming lipids is stabilized by one or more scaffold proteins. A “scaffold protein,” as used herein, is an amphipathic protein that can self-assemble with membrane-forming lipids in an aqueous environment, such as (substantially) encircling or encompassing the hydrophobic core of a lipid bilayer. Scaffold proteins generally have an alpha helical secondary structure, where several hydrophobic amino acids form a hydrophobic face and several hydrophilic amino acids form a hydrophilic face on the opposite side. The particle itself becomes water-soluble, and thus can be carried, e.g., in blood or lymph.

In some embodiments, the scaffold protein comprises, e.g., one or more of apolipoproteins, lipophorins, or derivatives or fragments thereof, in particular, derivatives or fragments that maintain capability to self-assemble with membrane-forming lipids. In some embodiments, the scaffold protein is a rationally designed protein or peptide. In some embodiment, the scaffold protein is a biological molecule produced by a living organism, such as a mammal, e.g., humans, non-human primates, rats, mice, rabbits, or guinea pigs. In specific embodiments, the scaffold protein is at least one of an apolipoprotein A (e.g., apo A-I, apo A-II, apo A-IV, apo A-V), apolipoprotein B (e.g., apo B48, apo B100), apolipoprotein C (e.g., apo C-I, apo C-II, apo C-HI, apo C-IV), apolipoprotein D, apolipoprotein H, apolipoprotein E (e.g., apoE2, apoE4), and/or lipophorin III. In some embodiments, the scaffold protein is a truncated version of an apolipoprotein capable of stabilizing a membrane bilayer, e.g., having a hydrophobic face and an opposite hydrophilic face. In a particular example, truncated apo4E is used, e.g., the 22Kd fragment of apoE4, termed “ApoE422k.” In some particular embodiments, the scaffold protein is human derived ApoE422k.

In some embodiments, the apolipoprotein is a truncated version of apoE3 (e.g. apoE322k), a truncated version of apoE2 (e.g. apoE222k), or a truncated version of apoA1 (e.g. A49ApoA1, MSP1, MSP1T2, MSP1E3D1). In some embodiments, the scaffold protein and membrane-forming lipid components are both biological molecules produced by a living organism, and form NLPs free of non-biologically derived materials.

One of skill will understand that the scaffold protein and membrane-forming lipids can be provided in suitable molar ratios to facilitate NLP assembly. In some embodiments, the scaffold protein and membrane-forming lipids are in a molar ratio of from 1:50 to 1:100; from 1:60 to 1:90; or from 1:70 to 1:80. In some embodiments, the scaffold protein and membrane-forming lipids are in a molar ratio of 1:60, 1:70, 1:75, 1:80, 1:85, 1:90, or 1:100. In a specific embodiment, the scaffold protein and membrane-forming lipids are in a molar ratio of 1:80. In some embodiments, a ratio is used such that most or all of the membrane-forming lipids arrange as bilayers, and none or few remain unassembled. In some embodiments, remaining unassembled lipids become stuck to reaction vessel walls, leaving none, few, or very few available to react with functional groups of a polypeptide.

In the present invention, the NLP will present one or more functionalized groups for interaction with a complementary functional group, e.g., on an antigen-binding polypeptide. In some embodiments, the functionalized group reacts with a complementary functional group on the polypeptide and does not conjugate to other components of the NLP, such as not conjugating to the scaffold protein(s). In some embodiments, less than 25% or 20-25%, less than 20% or 10-20%, less than 15% or 10-15%, less than 10% or 5-10%, less than 5% or 3-5%, or 0% of the functionalized groups conjugate to scaffold protein in a conjugate of the invention. In some embodiments, 5%, 4%, 3%, 2%, 1%, or 0% of the functionalized groups conjugate to scaffold protein in a conjugate of the invention.

Typically, not all of the membrane-forming lipids are functionalized. One of skill in the art will appreciate that amount of lipids with functionalized groups to be used will depend on, e.g., the identity of the functionalized group and/or the membrane-forming lipid, and/or the polypeptide to be conjugated therewith. For example, in some embodiments, less than 60%, e.g., 50-60%; less than 50%, e.g., 40-50%; less than 40%, e.g., 30-40%; less than 30%, e.g., 20-30%, or less than 20%, e.g., 10-20 of the membrane-forming lipids are functionalized. In specific embodiments, 10-40%, 15-35%, 20-35%, or 10%, 15%, 20%, 25%, 30%, or 35% of the membrane-forming lipids are functionalized. In a particular example, 20% of the membrane-forming lipids carry a functionalized group.

A “functional” or “functionalized” group refers to a group of atoms within a molecular structure that bring about the chemical reactions characteristic of that structure. For simplicity herein, the term “functionalized group” is used in reference to such groups on, or connected by a spacer to, a membrane-forming lipid of an NLP. The term “functional group” is used in reference to such groups on, or connected by a spacer to, a polypeptide, e.g., an antigen-binding polypeptide, for attaching to the NLP via one or more corresponding functionalized groups on the membrane-forming lipid. Examples of functional/functionalized groups include hydrocarbons, groups having double or triple bonds, halogen-containing groups, nitrogen-containing groups, oxygen-containing groups, and the like. In some embodiments, the functionalized group is selected from at least one group selected from an azide, amino, anhydride, alkyne, carboxy, halogens, hydroxyl, thiol, and phosphate group. Typically, a reaction between corresponding functional and functionalized groups results in bond formation and conjugation of the respective groups.

a. NLP Conjugation to a Polypeptide

The term “conjugation” or “binding” as used herein refers to an interaction between groups, in close proximity, resulting in a stable association between the groups. The interaction may involve covalent and/or noncovalent binding, e.g., electrostatic interactions. Conjugation can be accomplished using any number of functionalization strategies, through the use of one or more pairs of functionalized/functional groups, on the NLP lipids and the antigen-binding polypeptide, respectively, known to conjugate successfully under suitable conditions.

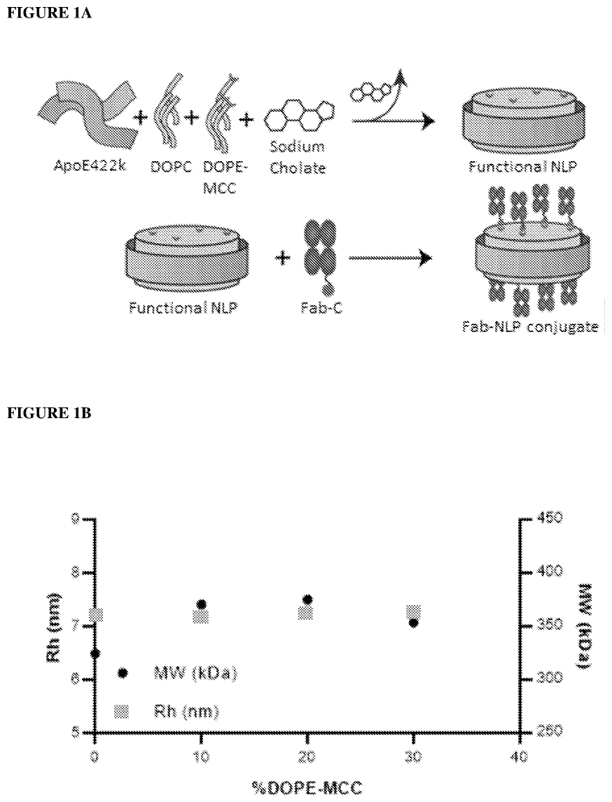

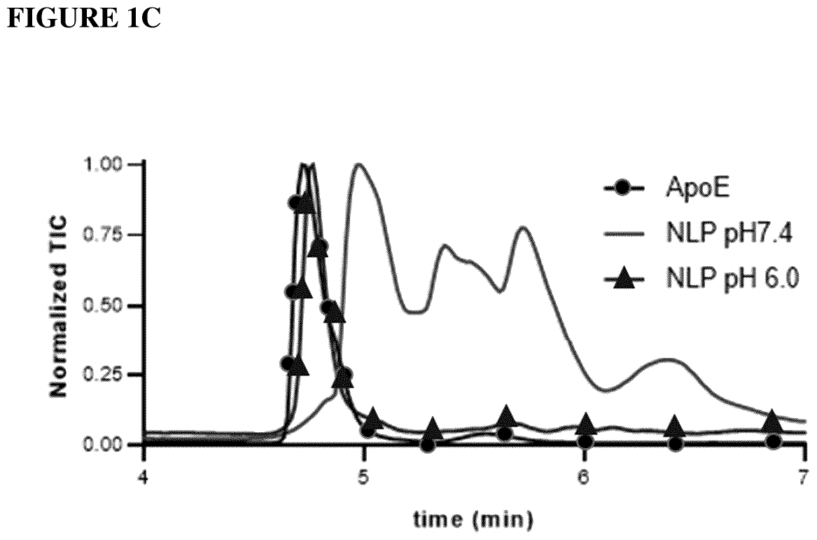

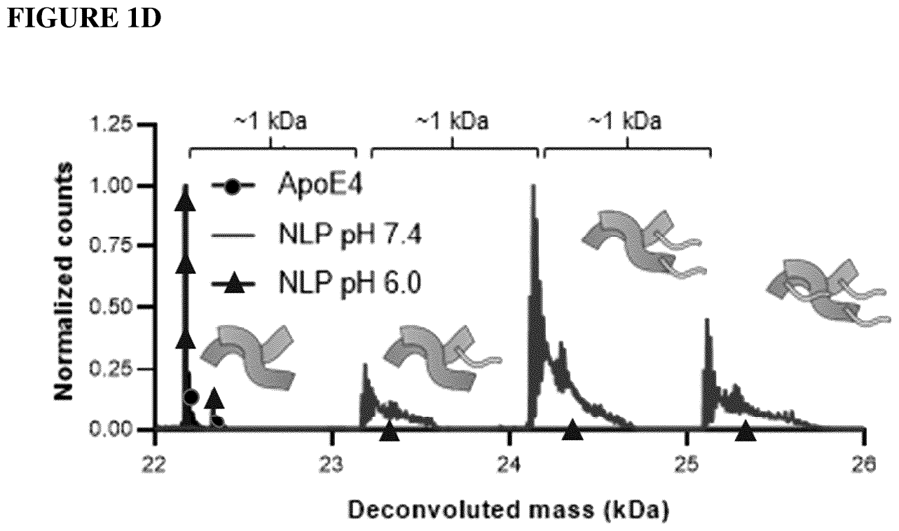

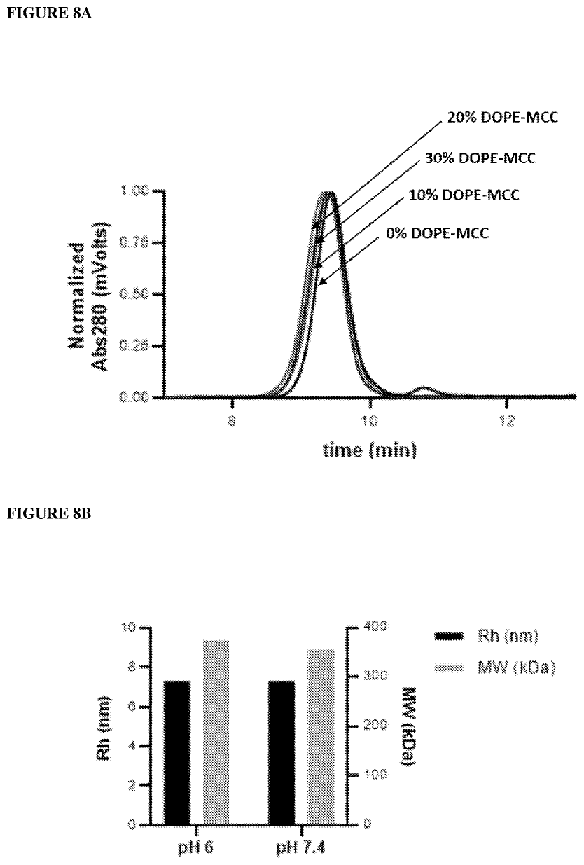

Successful conjugation of a polypeptide to an NLP, as described herein, can be confirmed or quantified using techniques known to those of skill in the art. Examples of techniques for characterizing conjugates of the present invention include, but are not limited to, fluorescence correlation spectroscopy, fourier transform infrared spectroscopy, size exclusion chromatography, surface plasmon resonance, raman spectroscopy, total internal reflection fluorescence, ultraspin filtration (see, e.g., Example 2, FIG. 8A, and FIG. 1B).

FIG. 1A depicts a schematic of assembly and conjugation of an NLP-Fab of the invention. In embodiments of the present invention, a nanolipoprotein particle is conjugated to at least one polypeptide, where the polypeptide is covalently attached, or otherwise bound or linked, to one or more components of the NLP, to form a NLP-polypeptide conjugate. The polypeptide generally attaches to one or more functionalized groups on the membrane-forming lipids, such as on the polar head, on one or both surfaces of the bilayer. The functionalized group may be any group capable of conjugating to a corresponding or complementary functional group on the polypeptide, as noted above. Examples of functionalized membrane-forming lipids include, but not limited to azide-bearing lipids, carboxylate-bearing lipids, maleimide-bearing lipids, chelated metal-bearing lipids, propargyl-bearing lipids, quaternary amine-bearing lipids, S-protein bearing lipids, and the like. Typically, the amount of functionalized membrane-forming lipids used during NLP assembly, compared to non-functionalized membrane-forming lipids, determines the amount of polypeptide that will conjugate to the NLP bilayer surface.

Examples of conjugating pairs of functionalized/functional groups include, but are not limited to, esters with amines; carboxylic acids with amines; azides with acetylenes; bivalent metals (e.g., Ni²⁺, Co²⁺, Cu²⁺, Zn²⁺) with poly-histidine; isothiocyanate with amine; avidin with biotin; glutathione S-transferase (GST) with glutathione; sulfhydryl with haloacetamide; sulfhydryls with pyridyl disulfides sulfhydryl with thiosulfate; and maleimides (or maleimide derivatives) with sulfhydryl. These components can be synthesized by known techniques and/or are commercially available, as one skilled in the art will recognize.

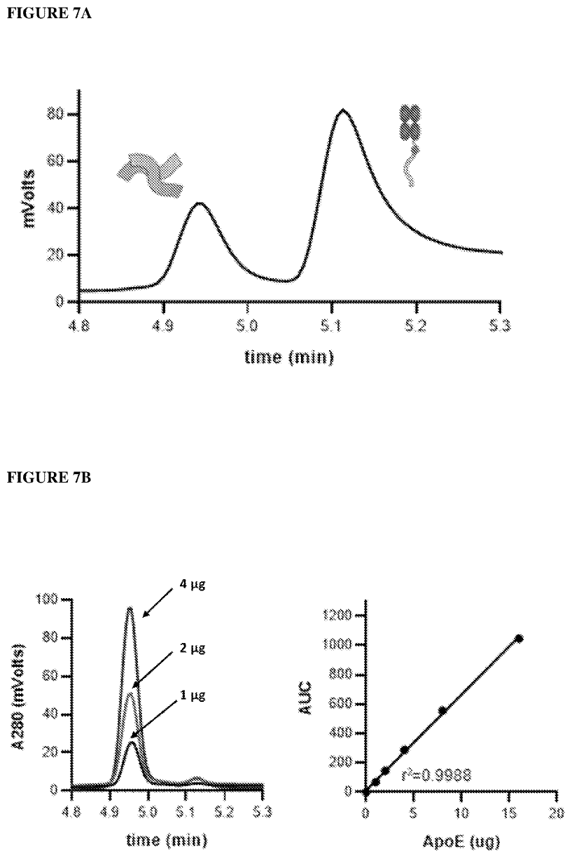

In specific embodiments, the functionalized group is one or more of maleimide derivatives, haloacetamides, pyridyldithio-propionates, and thiosulfates; and the complementary functional group is a free thiol group, such as a reduced sulfhydryl moiety. In a particular embodiment, the functionalized group is a maleimide and the complementary function group is a reduced cysteine residue. Maleimide bearing lipids are available commercially or can be synthesized by one skilled in the art. A specific example of a thiol-reactive membrane-forming lipid comprises 1,2-dihexadecanoyl-sn-glycero-3-phosphoethanolamine-N-[4-(p-maleimidophenyl) butyramide] (sodium salt). (See, e.g., Example 1, FIGS. 7A-7B).

In some embodiments, the functionalized group is directly located on, or part of the chemical structure of, the polar head of a membrane-forming lipid. In some embodiments, a spacer connects the functionalized group to the membrane-forming lipid. A “spacer” or “linker” may be composed of one or more linker components. Exemplary spacer components include 6-maleimidocaproyl (“MC”), maleimidopropanoyl (“MP”), p-aminobenzyloxycarbonyl (“PAB”), N-Succinimidyl 4-(2-pyridylthio) pentanoate (“SPP”), N-Succinimidyl 4-(N-maleimidomethyl) cyclohexane-1-carboxylate (“SMCC”), and N-Succinimidyl (4-iodo-acetyl) aminobenzoate (“SIAB”). Additional spacer components are known in the art and some are described herein. See also “Monomethylvaline Compounds Capable of Conjugation to Ligands,” U.S. Application Publication No. 2005/0238649, the contents of which are hereby incorporated by reference in its entirety.

In some embodiments, the spacer may comprise amino acid residues. Exemplary amino acid spacer components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). In some embodiments, the spacer comprises a peptide having a length of 1, 2, 3, 4, 5, 6, 7, 8, 9, 10, 11, 12, 13, 14, 15, 16, 17, 18, 19 or 20 amino acids. In some embodiments, the spacer is 2-6 amino acids, 3-5 amino acids, or 4 amino acids. The spacer is usually rich in glycine for flexibility, as well as serine or threonine for solubility. In some embodiments, a glycine-serine spacer is used, e.g., (GS)n, where G=glycine, S=serine, and n=2, 3, 4 or 5. In a particular embodiment, the spacer used for the Fab conjugation comprises or consists of the amino acid sequence GSGS. In some embodiments, other spacers are used that provide the similar flexibility and solubility as the GSGS spacer.

b. Antigen-Binding Polypeptide Components

In some embodiments of the NLP-polypeptide conjugates of the present invention, at least one antigen-binding polypeptide is covalently attached, or otherwise bound or linked, to one or more components of the NLP. Generally, the polypeptide is an antigen-binding polypeptide, such as a fragment of an antibody, e.g., a Fab, Fab′, Fab′-SH, F(ab′)₂, single chain Fab (scFab), single chain Fv (scFv), VH-VH dimer, VL-VL dimer, VH-VL dimer, single domain, diabody, or linear antibody. In some embodiments, the polypeptide is a Fab. In some embodiments, the antigen-binding polypeptide is a crossover Fab or a crossover single chain Fab. In some embodiments, the polypeptide is a “Fab-like molecule” or “Fab-like polypeptide,” that is a polypeptide having similar size and/or conformational shape as a Fab, though not necessarily capable of binding to an antigen.

In certain embodiments, the antigen-binding polypeptide comprises a hinge region (or part of the hinge region of an immunoglobulin), but does not include an Fc region, or does not include a full Fc region. In particular embodiments, the antigen-binding polypeptide comprises at least one Cys amino acid residue of a hinge region. In particular embodiments, a Cys residue provides the functional group, that is, provides a free thiol group for conjugation with functionalized groups on the NLP lipids. In specific embodiments, the antigen-binding polypeptide, e.g., a Fab, comprises at least one of Cys-226 and Cys-227 of the hinge region. In a particular embodiment, Cys-227 provides the free thiol group for conjugation.

As noted above, the polypeptide of NLP-polypeptide conjugates of the invention are covalently attached, or otherwise bound or linked, to the NLP by a functional group on one or more amino acid residues of the polypeptide. In some embodiments, the functional group is located C-terminally. Being “located C-terminally” means that the functional group is located towards the C-terminal end of the polypeptide. For example, the functional group may be located on the last amino acid residue in a polypeptide chain, considered from its N-terminus to its C-terminus. A C-terminally located group may be located on the second to last, third to last, fourth to last, or fifth to last amino acid residue, or on any one of the last 1-2, 1-3, 2-3, 1-4, 2-4, 1-5, 3-5, 1-6, 2-6, 3-6, 1-7, 3-7, 4-7, 1-8, 3-8, 5-8, or 1-10 amino acid residues at the C-terminus of a polypeptide.

In some embodiments, the functional group is located N-terminally. Being “located N-terminally” means that the functional group is located towards the N-terminal end of the polypeptide. For example, the functional group may be located on the first amino acid residue in polypeptide chain, considered from its N-terminus to its C-terminus. An N-terminally located group may be located on the second, third, fourth, or fifth amino acid residue, or on any of the first 1-2, 1-3, 2-3, 1-4, 2-4, 1-5, 3-5, 1-6, 2-6, 3-6, 1-7, 3-7, 4-7, 1-8, 3-8, 5-8, or 1-10 amino acid residues at the N-terminus of a polypeptide.

The functional group is selected to react with a corresponding or complementary functionalized group on a component of the NLP, e.g., on a membrane-forming lipid of the NLP, as described above. In some embodiments, the functional group is selected from the group consisting of maleimide derivatives, haloacetamides, pyridyldithio-propionates, and thiosulfates; and said complementary functional group is a free thiol group. In specific embodiments, the functional group is a thiol group, such as a cysteine thiol group. In some specific embodiments, the cysteine amino acid residue is a residue that formed a hinge disulfide bond in the antibody from which the antigen-binding polypeptide is derived. For example, the thiol group may be provided by at least one selected from Cys-226 and Cys-227 (based on Kabat numbering). In a particular embodiment, a C-terminal Cys-227, e.g., the C-terminal Cys-227 of a Fab, provides the free thiol group for conjugation with functionalized groups on the NLP lipids.

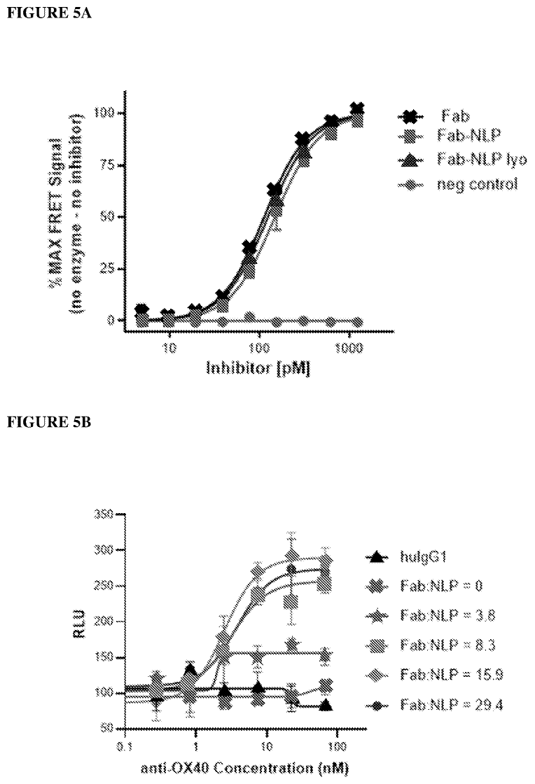

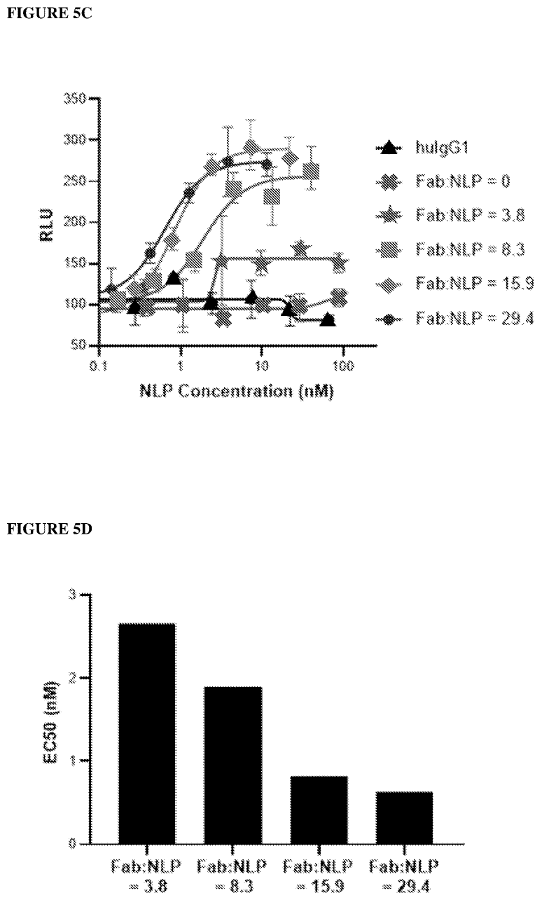

In some embodiments, the functional group is directly located on, or part of the chemical structure of, an amino acid residue of the antigen-binding polypeptide. In some embodiments, a spacer, as described above, connects the functional group to the antigen-binding polypeptide. In either format, the antigen-binding polypeptide, e.g., a Fab, conjugates to the NLP without loss, or without substantial loss, of antigen-binding activity, either in an inhibitory and agonist setting. See also Example 4, FIG. 5B, where site-specific Fab conjugation achieved about 30 Fab/NLP with minimal impact on NLP hydrodynamic radius, indicating that particle size is largely dictated by discoidal shape of the NLP.

Antigen-binding peptides are typically derived from an antibody molecule of one or more immunoglobulin classes. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, β, ε, γ, and μ, respectively. In some embodiments, the antigen-binding polypeptide is derived from an IgG molecule, e.g., a Fab of an IgG molecule.

In some aspects of the invention, the antigen-binding polypeptide is a therapeutic agent and the conjugate finds use as a delivery platform for the antigen-binding polypeptide. For example, the antigen-binding polypeptide (e.g., Fab) may function as the drug cargo or biologically active molecule, acting to neutralize a target, or block, agonize or antagonize a pathway. In some aspects of the invention, the antigen-binding polypeptide provides an antigen-targeted delivery platform. Attachment of a targeting Fab, e.g., can enrich drug delivery at the organ or tissue of interest via enhanced internalization (37, 38, 39). For example, the antigen-binding polypeptide (e.g., Fab) may recognize specific cell or disease markers, to direct the conjugates to specific targets. Conjugation of the antigen-binding polypeptide to an NLP can mediate targeting of the NLP. e.g., to facilitate delivery of the antigen-binding polypeptide, or an additional therapeutic or diagnostic agent, to the target. All of the references cited in this paragraph can be found below Example 5.

In certain embodiments, NLP-polypeptide conjugates of the present invention overcome drawbacks of previous nanolipoprotein approaches that have been developed for in vivo delivery of therapeutic drugs (9-12), diagnostic imaging agents (13), and vaccine and immunomodulation therapeutics (14-17). Over the past ten years, there have been efforts in the field of nanotechnology to address limitations of conventional drug delivery systems, including nonspecific biodistribution and targeting, poor aqueous solubility, limited oral bioavailability, and low therapeutic indices (1). Some approaches have included use of inorganic nanoparticles (2), polymeric based nanoparticles (3), polymeric micelles (4), dendrimers (5), liposomes (6), viral nanoparticles (7) and carbon nanotubes (6). Other strategies have included liposomes (33-35) and polymeric based nanoparticles (36). The NLP-conjugates of the present invention, however, offer several distinct advantages over other nanoparticle-based delivery technologies, including low toxicity and low immunogenicity (20). For example, in some embodiments, the NLP-polypeptide conjugates are free of non-biologically derived materials, comprising lipids, scaffold proteins, and antigen-binging polypeptides that are produced naturally and/or are derived from naturally produced materials, and thereby illicit lower immune response and/or are less toxic than previous delivery platforms requiring a non-biologically derived component. Other advantages, in certain embodiments, include good stability, e.g., without use of crosslinkers; good manufacturability, including feasible production of highly concentrated but low viscosity formulations, minimal to no inter-particle crosslinking, a relatively homogeneous NLP-polypeptide population, and/or enhanced antigen-binding potency, as discussed herein. All of the references cited in this paragraph can be found below Example 5.

In some embodiments, the antigen-binding polypeptide (e.g., a Fab) binds one or more tumor specific antigens, or tumor markers, directing the conjugates to a tumor. For example, epidermal growth factor receptor (EGFR) is overexpressed by certain tumor cells. In an in vivo xenograft tumor model that overexpresses EGFR, anti-EGFR immunoliposomes achieved cellular uptake that was 6 times greater than nontargeted liposomes after a single dose measured 24 hours post injection. Further, anti-EGFR immunoliposome-doxorubicin (a potent cytotoxin) showed significant tumor regression, greater than doxorubicin alone or untargeted liposome-doxorubicin.

Other examples of tumor markers that naturally occur on the surface of tumor cells comprise, but are not limited to, FAP (fibroblast activation protein), CEA (carcinoembryonic antigen), p95 (p95HTER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52Flt3, folate receptor 1 (FOLR1), human trophoblast cell-surface antigen 2 (Trop-2) cancer antigen 12-5 (CA-12-5), human leukocyte antigen-antigen D related (HLA-DR), MUC-1 (Mucin-1), A33-antigen, PSMA (prostate-specific membrane antigen), FMS-like tyrosine kinase 3 (FLT-3), PSMA (prostate specific membrane antigen), PSCA (prostate stem cell antigen), transferrin-receptor, TNC (tenascin), carbon anhydrase IX (CA-IX), and/or peptides bound to a molecule of the human major histocompatibility complex (MHC).

Many such tumor antigens are known in the art or readily obtained by the skilled artisan. For example, the sequence(s) of the (human) members of the A33-antigen, BCMA (B-cell maturation antigen), cancer antigen 12-5 (CA-12-5), carbon anhydrase IX (CA-IX), CD19, CD20, CD22, CD33, CD38, CEA (carcinoembryonic antigen), EpCAM (epithelial cell adhesion molecule), FAP (fibroblast activation protein), FMS-like tyrosine kinase 3 (FLT-3), folate receptor 1 (FOLR1), HER-1 (human epidermal growth factor 1), HER-2 (human epidermal growth factor 2), HER-3 (human epidermal growth factor 3), human leukocyte antigen-antigen D related (HLA-DR), MSLN (mesothelin), MCSP (melanoma chondroitin sulfate proteoglycan), MUC-1 (Mucin-1), PSMA (prostate specific membrane antigen), PSMA (prostate-specific membrane antigen), PSCA (prostate stem cell antigen), p95 (p95HER2), transferrin-receptor, TNC (tenascin), human trophoblast cell-surface antigen 2 (Trop-2) are available in the UniProtKB/Swiss-Prot database and can be retrieved from http://www.uniprot.org/uniprot/?query=reviewed %3Ayes. The skilled person may easily deduce the relevant coding region of these (protein) sequences in these databank entries, which may also comprise genomic DNA as well as mRNA/cDNA.

The sequence(s) of the (human) FAP (fibroblast activation protein) can be obtained from the Swiss-Prot database entry Q12884 (entry version 168, sequence version 5); the sequence(s) of the (human) CEA (carcinoembryonic antigen) can be obtained from the Swiss-Prot database entry P06731 (entry version 171, sequence version 3); the sequence(s) of the (human) EpCAM (Epithelial cell adhesion molecule) can be obtained from the Swiss-Prot database entry P16422 (entry version 117, sequence version 2); the sequence(s) of the (human) MSLN (mesothelin) can be obtained from the UniProt Entry number Q 13421 (version number 132; sequence version 2); the sequence(s) of the (human) FMS-like tyrosine kinase 3 (FLT-3) can be obtained from the Swiss-Prot database entry P36888 (primary citable accession number) or Q13414 (secondary accession number) with the version number 165 and the sequence version 2; the sequences of (human) MCSP (melanoma chondroitin sulfate proteoglycan) can be obtained from the UniProt Entry number Q6UVK1 (version number 118; sequence version 2); the sequence(s) of the (human) folate receptor 1 (FOLR1) can be obtained from the UniProt Entry number P15328 (primary citable accession number) or Q53EW2 (secondary accession number) with the version number 153 and the sequence version 3; the sequence(s) of the (human) trophoblast cell-surface antigen 2 (Trop-2) can be obtained from the UniProt Entry number P09758 (primary citable accession number) or Q15658 (secondary accession number) with the version number 172 and the sequence version 3; the sequence(s) of the (human) PSCA (prostate stem cell antigen) can be obtained from the UniProt Entry number 043653 (primary citable accession number) or Q6UW92 (secondary accession number) with the version number 134 and the sequence version 1; the sequence(s) of the (human) HER-1 (Epidermal growth factor receptor) can be obtained from the Swiss-Prot database entry P00533 (entry version 177, sequence version 2); the sequence(s) of the (human) HER-2 (Receptor tyrosine-protein kinase erbB-2) can be obtained from the Swiss-Prot database entry P04626 (entry version 161, sequence version 1); the sequence(s) of the (human) HER-3 (Receptor tyrosine-protein kinase erbB-3) can be obtained from the Swiss-Prot database entry P21860 (entry version 140, sequence version 1); the sequence(s) of the (human) CD20 (B-lymphocyte antigen CD20) can be obtained from the Swiss-Prot database entry P11836 (entry version 117, sequence version 1); the sequence(s) of the (human) CD22 (B-lymphocyte antigen CD22) can be obtained from the Swiss-Prot database entry P20273 (entry version 135, sequence version 2); the sequence(s) of the (human) CD33 (B-lymphocyte antigen CD33) can be obtained from the Swiss-Prot database entry P20138 (entry version 129, sequence version 2); the sequence(s) of the (human) CA-12-5 (Mucin 16) can be obtained from the Swiss-Prot database entry Q8WX17 (entry version 66, sequence version 2); the sequence(s) of the (human) HLA-DR can be obtained from the Swiss-Prot database entry Q29900 (entry version 59, sequence version 1); the sequence(s) of the (human) MUC-1 (Mucin-1) can be obtained from the Swiss-Prot database entry P15941 (entry version 135, sequence version 3); the sequence(s) of the (human) A33 (cell surface A33 antigen) can be obtained from the Swiss-Prot database entry Q99795 (entry version 104, sequence version 1); the sequence(s) of the (human) PSMA (Glutamate carboxypeptidase 2) can be obtained from the Swiss-Prot database entry Q04609 (entry version 133, sequence version 1), the sequence(s) of the (human) transferrin receptor can be obtained from the Swiss-Prot database entries Q9UP52 (entry version 99, sequence version 1) and P02786 (entry version 152, sequence version 2); the sequence of the (human) TNC (tenascin) can be obtained from the Swiss-Prot database entry P24821 (entry version 141, sequence version 3); or the sequence(s) of the (human) CA-IX (carbonic anhydrase IX) can be obtained from the Swiss-Prot database entry Q16790 (entry version 115, sequence version 2). The skilled person may easily deduce the relevant coding region of these (protein) sequences in these databank entries, which may also comprise genomic DNA as well as mRNA/cDNA.

Conjugates of the present invention can provide one or more antigen-binding polypeptides, e.g., as described herein or known in the art, in multimeric and/or multivalent formats, described in more detail below.

c. Multimeric Formats

Conjugation of a polypeptide (e.g., antigen-binding polypeptide) to NLPs, as described herein, can facilitate multimerization of the antigen-binding polypeptide, e.g., where conjugates provide two or more molecules of the antigen-binding polypeptide, that is, where multiple copies of a same molecule are attached to a NLP. This format can increase activity or avidity of the antigen-binding polypeptide, in a defined way. For example, in certain antibody therapeutic modalities, valency can be a primary limitation and there has been a renewed focus on known mAb targets that require valencies beyond two for a pathway to be potently agonized or antagonized. Examples of such targets include TNF family members, such as DR5 and OX40, which require receptor clustering for activation (41, 42). While various engineering strategies have been developed to expand valencies beyond two, e.g., using both IgG-like and non-IgG-like formats (41, 42), such as triabodies, tetrabodies, pentabodies and self-assembling hexameric IgG antibodies (43-45), these formats have several limitations. For example, previously used formats can negatively impact pharmacokinetics, as well as failing to achieve valencies beyond 3-6 (43) and thus, in many cases, fail to achieve sufficient agonizing/antagonizing activity. The present invention, in certain aspects, provides platforms for delivering a high density of antigen-binding polypeptides, e.g., Fabs, enabling in vivo potency needed to attain clinically relevant therapeutic index in connection with certain targets. All of the references cited in this paragraph can be found below Example 5.

Another aspect of the present invention relates to increasing avidity, activity, or potency of an antigen-binding polypeptide, using the conjugate platform described herein, where the antigen-binding polypeptide of an NLP-polypeptide conjugate of the present invention is directed to a target requiring valencies beyond two. For example, the target may be one or more selected from the group consisting of TNF family members, DR4, OX40, GITR, Tie2, factor D, VEGF, MerTK, CD3, and Lymphotoxin beta receptor. In a specific embodiment, the antigen-binding polypeptide is a Fab that binds OX40. In a specific embodiment, the antigen-binding polypeptide is a Fab that binds DR4. In some embodiments, the antigen-binding polypeptide is at least one Fab selected from anti-OX40, anti-GITR, anti-Tie2, anti-factor D, anti-VEGF, anti-MERTK, anti-CD3, anti-Lymphotoxin beta receptor, and anti-DR4 Fab.

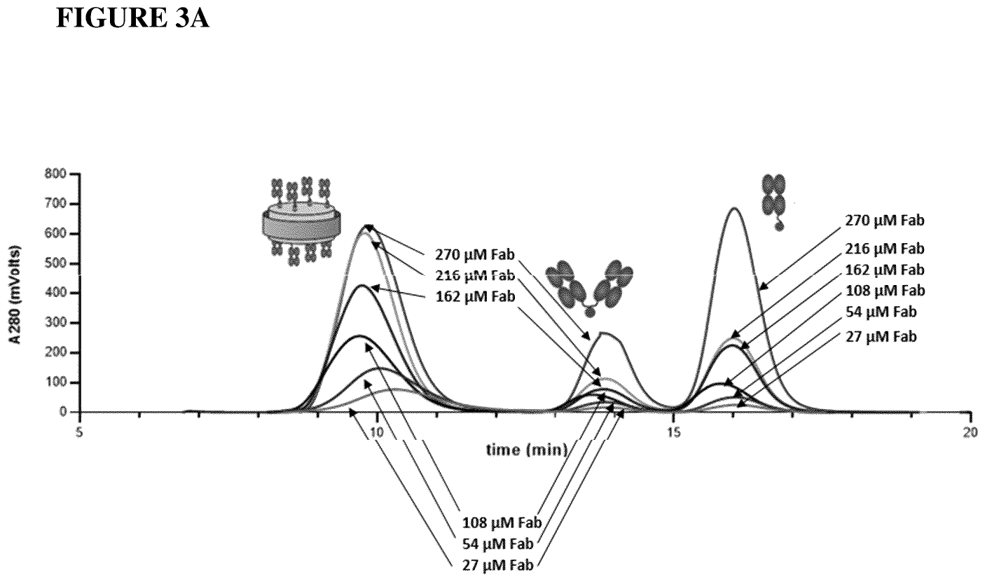

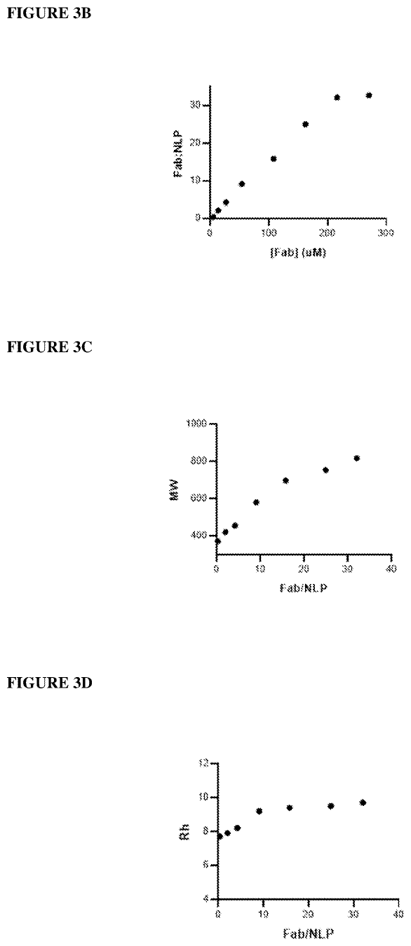

In some embodiments, an NLP-polypeptide conjugate of the present invention provides two or more molecules of an antigen-binding polypeptide, e.g., a Fab, such as 2-60 molecules per conjugate, to increase activity or avidity of the antigen-binding polypeptide. In some embodiments, the conjugate comprises 2-60, 3-50, 4-40, 5-35, 6-30, or 7-20 molecules of antigen-binding polypeptide e.g., a Fab. In particular embodiments, the conjugate comprises 2-32, 6-32, 10-30, 10-60, 15-30, or 20 molecules of antigen-binding polypeptide, e.g., a Fab (see, e.g., Example 2, FIGS. 3A-3D). In some specific embodiments, the conjugate comprises 3, 4, 5, 6, 7, 8, 9, 10, 11, or 12 molecules of antigen-binding polypeptide, e.g., a Fab. Such embodiments generally increase activity and/or avidity of the antigen-binding polypeptide. For example, a conjugate having 4-8 molecules of an anti-OX40 Fab shows increased anti-XO40 activity (see, e.g., Example 4, FIG. 5B).

In some embodiments, conjugation of a polypeptide (e.g., an antigen binding polypeptide, such as a Fab) to NLPs, as described herein, increases stability of the antigen-binding polypeptide and/or the NLP. For example, conjugation of an antigen-binding polypeptide (e.g., a Fab or Fab-like molecule to an NLP can improve stability of an antigen-binding polypeptide (e.g., a Fab or Fab-like molecule) in vitro, such as increasing shelf life; and/or in vivo, such as improving serum half-life. In some embodiments, conjugation of the NLP to an antigen-binding polypeptide (e.g., a Fab or Fab-like molecule) improves stability of the NLP particle in vitro, such as increasing shelf life; and/or in vivo, such as improving serum half-life. Accordingly, another aspect of the present invention relates increasing stability and/or half-life of an antigen-binding polypeptide (e.g., a Fab or Fab-like molecule) or of a nanolipoprotein particle, using the conjugate platform described herein.

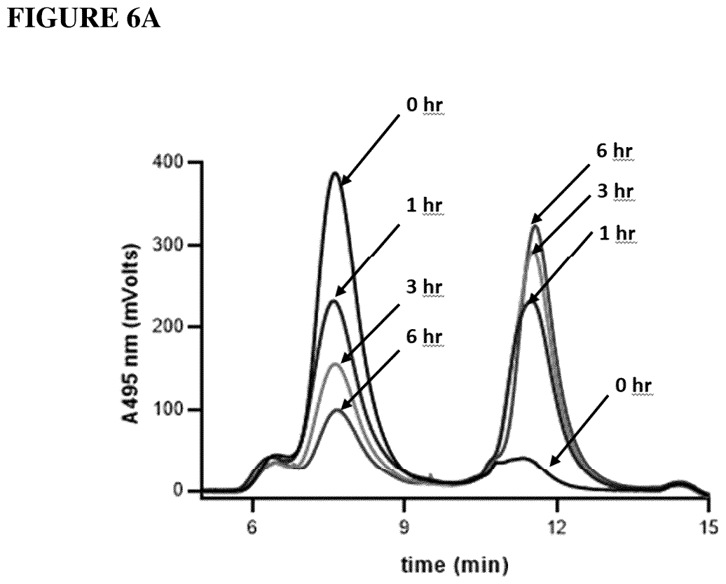

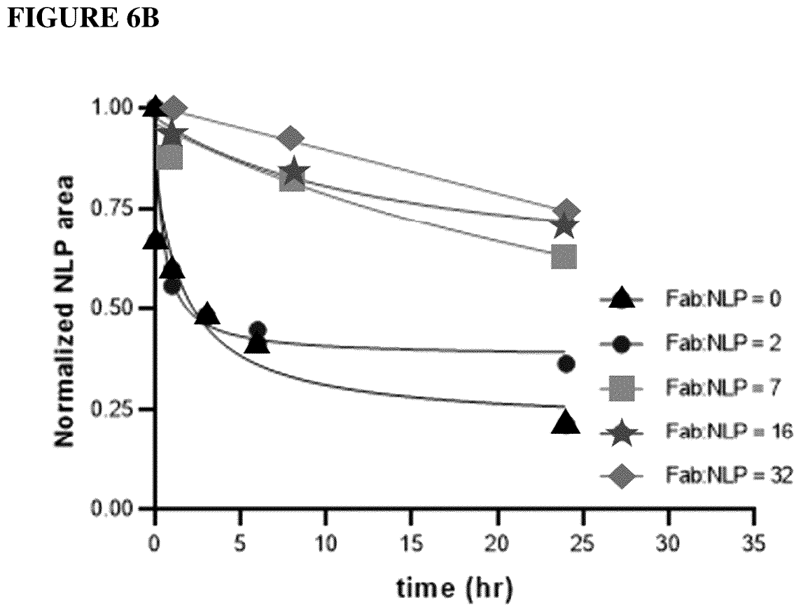

In some embodiments, conjugation of a polypeptide (e.g., an antigen binding polypeptide, such as a Fab or a Fab-like molecule) to NLPs, as described herein, increases stability of the polypeptide-NLP complex (e.g., the antigen-binding polypeptide-NLP complex). In some embodiments, stability of the polypeptide-NLP complex (e.g., the antigen-binding polypeptide-NLP complex) is increased by 30-40%, 40-50%, 50-60%, 60-70%, 70-80%, or 80-90% as compared to an NLP that is not conjugated to a polypeptide (e.g., an antigen binding polypeptide such as a Fab or Fab-like molecule). In some embodiments, stability of the polypeptide-NLP complex (e.g., the antigen-binding polypeptide-NLP complex) is increased by 30-40%, 40-50%, 50-60%, 60-70%, 70-80%, or 80-90% as compared to a polypeptide (e.g., an antigen binding polypeptide such as a Fab or Fab-like molecule) that is not conjugated to an NLP. In some embodiments, stability of a conjugate comprising an NLP and antigen-binding polypeptides (e.g., a Fab or Fab-like molecule) is increased by 30-40%, 40-50%, 50-60%, 60-70%, 70-80%, or 80-90%, e.g., as measured herein, as compared to an NLP that does not comprise antigen-binding polypeptides. Stable NLP-polypeptides conjugates generally include 5-60, 10-60, 15-30, or 20 molecules of antigen-binding polypeptide per conjugate, including 7-60, 7-40, 7-32, or 8-30 antigen-binding polypeptides (e.g., Fabs) per conjugate. In specific examples, the NLP-polypeptide conjugate comprises 6, 7, 8, 9, or 10 antigen-binding polypeptides (e.g., Fabs). In certain embodiments, the antigen-binding polypeptide is a Fab or Fab-like molecule. In a particular example, the NLP-polypeptide conjugate includes 7-32 Fab molecules per conjugate. (see, e.g., Example 5, FIGS. 6A-6B, demonstrating the effect of Fab loading on Fab-NLP stability in a complex biological matrix (50% Sera) at a physiologically relevant temperature (37° C.), measured by analytical SEC, where Fab-NLPs demonstrated increased stability, with >63% of Fab-NLP remaining intact after 24 hours at Fab per particle ratios of 7 or greater).

Accordingly, the present invention provides, in some embodiments, NLP-polypeptide conjugates that are surprisingly stable and help overcome pharmacokinetic problems of other nanoparticle delivery platforms. For example, the in vivo half-lives of nanoparticles are known to be about 1-3 days (see reference 40 below Example 5), and monoclonal antibody therapeutics often rely on Fc-mediated pharmacokinetics to extend half-live via FcRn recycling. NLP-polypeptide conjugates of the present invention, in some embodiments, surprising improve NLP stability, without Fc-mediated pharmacokinetics, based on incorporation of multiple copies of polypeptides, e.g., Fabs or Fab-Like Molecules onto the NLP Discoidal Surfaces.

d. Multispecific Formats

Another aspect of the invention provides multi-specific constructs, i.e., NLP conjugates comprising one or more antigen-binding polypeptides (e.g., Fabs or Fab-like molecules) that bind two or more targets. In some embodiments, the targets comprise different antigens. In some embodiments, the targets comprise the same antigen. In some embodiments, the targets comprise different epitopes on the same antigen. In some embodiments, the conjugate comprises 2, 3, 4, 5, 6, 7, or 8 different antigen-biding polypeptides, e.g., 2, 3, 4, 5, 6, 7, or 8 different Fabs that bind 2, 3, 4, 5, 6, 7, or 8 different antigens or epitopes. In some embodiments, the conjugate comprises 10-12, 10-15, 10-20, 15-20, 15-25, 15-30, or 20-30 different antigen-biding polypeptides, e.g., 10-12, 10-15, 10-20, 15-20, 15-25, 15-30, or 20-30 different Fabs that bind 10-12, 10-15, 10-20, 15-20, 15-25, 15-30, or 20-30 different antigens or epitopes. In some embodiments, the conjugate comprises 25-30, 25-50, 30-45, 30-50, 40-50, 30-60, or 40-60 different antigen-biding polypeptides, e.g., 25-30, 25-50, 30-45, 30-50, 40-50, 30-60, or 40-60 different Fabs that bind 25-30, 25-50, 30-45, 30-50, 40-50, 30-60, or 40-60 different antigens or epitopes.

In some embodiments, the antigen-binding polypeptides of an NLP-polypeptide conjugate of the invention are directed to two different targets to give a bi-specific conjugate. While the idea of bispecific antibodies is more than 60 years old, challenges associated with design, stability, and manufacturing originally delayed their development. More recently, advances in antibody engineering technologies have led to growing interest in bispecific modalities, resulting in recent FDA approval of three bispecific antibody therapeutics (Removab, targeting EpCAM and CD3; Blincyto, targeting CD19 and CD3; and Hemlibra (Chugai), targeting Factor IXa and Factor X), as well as over 100 multi-specific candidates in clinical trials (e.g., Faricimab, targeting VEGF-A and Ang-2). A bi-specific construct facilitates unique mechanisms of action, such as engaging immune cells to tumor cells, blocking signaling pathways, and/or delivering payloads specifically to tumors.

The NLP-polypeptide conjugates disclosed herein provide a novel and versatile bispecific platform, which can readily be expanded to multi-specific formats and/or high valencies. For example, in some embodiments, the NLP-polypeptide conjugate comprises one molecule of each of two different Fabs, providing a bivalent, bispecific conjugate. In some embodiments, the NLP-polypeptide conjugate comprises two molecules of each of two different Fabs, providing a tetravalent, bispecific conjugate. In some embodiments, the NLP-polypeptide conjugate comprises one molecule of a first Fab, and two molecules of a second Fab, providing a trivalent, bispecific conjugate. In some embodiments, the NLP-polypeptide conjugate comprises three copies of each of two different Fabs, providing a hexavalent, bispecific conjugate. In some embodiments, the NLP-polypeptide conjugate comprises two copies of each of three different Fabs, providing a hexavalent, trispecific conjugate. In some embodiments, the NLP-polypeptide conjugate comprises two copies of a first Fab, and three copies of a second Fab, providing a pentavalent, bispecific conjugate. In some embodiments, the NLP-polypeptide conjugate comprises four copies of each of two different Fabs, providing an octavalent, bispecific conjugate. Additional configurations for providing specific multi-valencies and multi-specificities will be apparent to those of skill in the art.

In some embodiments, the NLP-polypeptide conjugate comprises at least one molecule of each of two antigen-binding polypeptides that bind a pair of clinically relevant targets. A “pair of clinically relevant targets,” a “clinically relevant pair of target antigens,” a “clinically relevant pair,” or similar expressions herein, refer to two disease mediators (such as cell surface receptors, soluble ligands, or other proteins), that play a role in the pathophysiology of a particular disease, such that binding by a bi-specific antibody, or other bispecific construct, to the two targets can result in a beneficial effect in treating the disease. For example, when both a T cell and a tumor cell are bound, e.g., by a bispecific NLP-polypeptide conjugate of the present invention, a cytolytic synapse is formed, in which the T cells release pore-forming perforin and cytotoxic granzyme-B, in turn leading to death of the target cell. As another example, a bispecific NLP-polypeptide conjugate described herein can simultaneously block signaling pathways of two targets with one molecule, which in advantageous in inhibiting complex pathways such as angiogenesis.

In some embodiments, the clinically relevant antigen pair comprises a T cell marker and a tumor antigen, such as any one or more of the tumor antigens disclosed herein and/or known in the art. In some embodiments, the clinically relevant antigen pair comprises a co-stimulatory receptor and a tumor antigen, such as any one or more of the tumor antigens disclosed herein and/or known in the art. In some embodiments, the clinically relevant antigen pair comprises a natural killer (NK) cell marker and a tumor antigen, such as any one or more of the tumor antigens disclosed herein and/or known in the art.

A “T-cell marker” refers to any target that can be recognized by an antigen-binding moiety, and that preferentially directs the antigen-binding moiety to T-cells over other cells or tissues of a subject, following administration of the antigen-binding moiety to the subject. An exemplary T-cell marker comprises CD3. A “NK cell marker” refers to any target that can be recognized by an antigen-binding moiety, and that preferentially directs the antigen-binding moiety to T-cells over other cells or tissues of a subject, following administration of the antigen-binding moiety to the subject. An exemplary NK cell markers comprises CD316A.

Additional examples of molecules that may be targeted by a bispecific or multi-specific conjugate (e.g., a NLP-Fab conjugate or a NLP-Fab-like molecule conjugate) provided herein include, but are not limited to, cytokines, soluble serum proteins and their receptors, and other membrane bound proteins (e.g., adhesins). In specific embodiments, the bi- or multi-specific conjugate (e.g., a NLP-Fab conjugate or a NLP-Fab-like molecule conjugate) binds one, two, or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (PFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSILON), FEL1 (ZETA), IL 1A, IL 1B, IL2, IL3, IL4, IL5, IL6, IL7, IL8, IL9, IL10, IL11, IL 12A, IL 12B, IL 13, IL 14, IL 15, IL 16, IL 17, IL 17B, IL 18, IL 19, IL20, IL22, IL23, TL24, TL25, IL26, IL27, IL28A, IL28B, TL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, Il0RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.

In some embodiments, the target antigen is a chemokine, chemokine receptor, or a chemokine-related protein. In specific embodiments, the bi- or multi-specific conjugate binds one, two, or more chemokines, chemokine receptors, or chemokine-related proteins selected from the group consisting of CCL1 (1-309), CCL2 (MCP-1/MCAF), CCL3 (MIP-Iα), CCL4 (MIP-10), CCL5 (RANTES), CCL7 (MCP-3), CCL8 (mcp-2), CCL11 (cotaxin), CCL 13 (MCP-4), CCL 15 (MIP-1δ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2, eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-1β), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP 10, CKLFSF2, CKLFSF3, CKLFSF4, CKLFSF5, CKLFSF6, CKLFSF7, CKLFSF8, BDNF, C5R1, CSF3, GRCC10 (C10), EPO, FY (DARC), GDF5, HDF1, HDF1α, DL8, PRL, RGS3, RGS13, SDF2, SL1T2, TLR2, TLR4, TREM1, TREM2, and VHL.

In some embodiments, the target antigen is a CD protein. In specific embodiments, the bi- or multi-specific conjugate (e.g., a NLP-Fab conjugate or a NLP-Fab-like molecule conjugate) binds one, two, or more CD proteins selected from the group consisting of CD3, CD4, CD5, CD16, CD19, CD20, CD34; CD64, CD200 members of the ErbB receptor family such as the EGF receptor, HER2, HER3 or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-S, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mp1 receptor; CTLA-4; RANKL, RANK, RSV F protein, protein C, etc.

In particular embodiments, the multi-specific NLP-polypeptide conjugate (e.g., a NLP-Fab conjugate or a NLP-Fab-like molecule conjugate) binds low density lipoprotein receptor-related protein (LRP)-1 or LRP-8 or transferrin receptor, and at least one target selected from the group consisting of 1) beta-secretase (BACE1 or BACE2), 2) alpha-secretase, 3) gamma-secretase, 4) tau-secretase, 5) amyloid precursor protein (APP), 6) death receptor 6 (DR6), 7) amyloid beta peptide, 8) alpha-synuclein, 9) Parkin, 10) Huntingtin, 11) p75 NTR, and 12) caspase-6.

In particular embodiments, the multi-specific NLP-polypeptide conjugate (e.g., a NLP-Fab conjugate or a NLP-Fab-like molecule conjugate) binds one or more of the clinically relevant pairs selected from the group consisting of: IL-1 alpha and IL-1 beta: IL-12 and IL-1S; IL-13 and IL-9; IL-13 and IL-4; IL-13 and IL-5; IL-5 and IL-4; IL-13 and IL-1beta; IL-13 and IL-25; IL-13 and TARC; IL-13 and MDC; IL-13 and MEF; IL-13 and TGF; IL-13 and LHR agonist; IL-12 and TWEAK; IL-13 and CL25; IL-13 and SPRR2a; IL-13 and SPRR2b; IL-13 and ADAMS; IL-13 and PED2; IL17A and IL17F; CD3 and CD19; CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3; TNF-alpha and TGF-beta; TNF-alpha and IL-1 beta; TNF-alpha and IL-2; TNF-alpha and IL-3; TNF-alpha and IL-4; TNF-alpha and IL-5; TNF-alpha and IL-6; TNF-alpha and IL8; TNF-alpha and IL-9; TNF-alpha and IL-10, TNF-alpha and IL-11; TNF-alpha and IL-12; TNF-alpha and IL-13; TNF-alpha and IL-14; TNF-alpha and IL-15; TNF-alpha and IL-16; TNF-alpha and IL-17; TNF-alpha and IL-18; TNF-alpha and IL-19; TNF-alpha and IL-20; TNF-alpha and IL-23; TNF-alpha and IFN-alpha; TNF-alpha and CD4; TNF-alpha and VEGF; TNF-alpha and MIF; TNF-alpha and ICAM-1; TNF-alpha and PGE4; TNF-alpha and PEG2; TNF-alpha and RANK ligand; TNF-alpha and Te38; TNF-alpha and BAFF; TNF-alpha and CD22; TNF-alpha and CTLA-4; TNF-alpha and GP130; TNF-alpha and IL-12p40; VEGF and HER2; VEGF-A and HER2; VEGF-A and PDGF; HER1 and HER2; VEGFA and ANG2; VEGF-A and VEGF-C; VEGF-C and VEGF-D; HER2 and DR5; VEGF and IL-8; VEGF and MET; VEGFR and MET receptor; EGFR and MET; VEGFR and EGFR; HER2 and CD64; HER2 and CD3; HER2 and CD16; HER2 and HER3; EGFR (HER I) and HER2; EGFR and HER3; EGFR and HER4; IL-14 and IL-13; IL-13 and CD40L; IL4 and CD40L; TNFR1 and IL-1R; TNFR1 and IL-6R; TNFR1 and IL-18R; EpCAM and CD3; MAPG and CD28; EGFR and CD64; CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

Additional clinically relevant pairs that may be co-targeted by multi-specific NLP-polypeptide conjugates of the present invention (e.g., a NLP-Fab conjugate or a NLP-Fab-like molecule conjugate) include CD63 and CD95 (a death receptor); HER2 and CD63 (a receptor involved in lysosomal internalization); CD20 or CD19 and CD47 (interrupting the “Don't cat me” signal); LAG-3 and PD1; CTLA4 and PD1; and LAG-3 and PD-L1.

In a specific embodiment, the NLP-polypeptide conjugate comprises a first antigen-binding polypeptide (e.g., a first Fab) that binds CD3 (a general T-cell marker) and at a second antigen-binding polypeptide (e.g., a second Fab) that binds targeting CD19 (a tumor antigen). In another specific embodiment, the NLP-polypeptide conjugate comprises a first antigen-binding polypeptide (e.g., a first Fab) that binds CD3; and a second antigen-binding polypeptide (e.g., a second Fab) that binds EpCAM (a tumor antigen). In still another specific embodiment, the NLP-polypeptide conjugate comprises a first antigen-binding polypeptide (e.g., a first Fab) that binds CD3; and a second antigen-binding polypeptide (e.g., a second Fab) that binds CEA. In yet still another specific embodiment, the NLP-polypeptide conjugate comprises a first antigen-binding polypeptide (e.g., a first Fab) that binds CD16A (a NK cell marker) and a second antigen-binding polypeptide (e.g., a second Fab) that binds CD30 or CD33; and optionally a third antigen-binding polypeptide (e.g., a third Fab) that binds IL-15.

In one particular example, the NLP-polypeptide conjugate comprises a first antigen-binding polypeptide (e.g., a first Fab) that binds Ang-2; and a second antigen-binding polypeptide (e.g., a second Fab) that binds VEGF-A. In another particular example, the NLP-polypeptide conjugate comprises a first antigen-binding polypeptide (e.g., a first Fab) that binds delta-like ligand 4; and a second antigen-binding polypeptide (e.g., a second Fab) that binds VEGF. In still another particular example, the NLP-polypeptide conjugate comprises a first antigen-binding polypeptide (e.g., a first Fab) that binds DR5; and a second antigen-binding polypeptide (e.g., a second Fab) that binds FAP (fibroblast activation protein).

In some particular embodiments, the NLP-polypeptide conjugate comprises a first antigen-binding polypeptide (e.g., a first Fab) that binds CD3; and a second antigen-binding polypeptide (e.g., a second Fab) that binds at least one of CD33, gp100, HER2, glypican-3, and TROP-2; and optionally a third antigen-binding polypeptide (e.g., a third Fab) that binds at least one co-stimulatory molecule selected from IL-2, CD137, and CD28. In some particular embodiments, the NLP-polypeptide conjugate comprises a first antigen-binding polypeptide (e.g., a first Fab) that binds CD3; and a second antigen-binding polypeptide (e.g., a second Fab) that binds at least one of CD33, gp100, HER2, glypican-3, and TROP-2; and optionally a third antigen-binding polypeptide (e.g., a third Fab) that binds PD1 and/or PD-L1. In a particular embodiment, the NLP-polypeptide conjugate comprises a first antigen-binding polypeptide (e.g., a first Fab) that binds CD3; a second antigen-binding polypeptide (e.g., a second Fab) that binds CEA; and a third antigen-binding polypeptide (e.g., a third Fab) that binds at least one of PD1 and PD-L1.

In some particular embodiments, the NLP-polypeptide conjugate comprises a first antigen-binding polypeptide (e.g., a first Fab) that binds CD28; and a second antigen-binding polypeptide (e.g., a second Fab) that binds CD20. In some particular embodiments, the NLP-polypeptide conjugate comprises a first antigen-binding polypeptide (e.g., a first Fab) that binds PD1; and a second antigen-binding polypeptide (e.g., a second Fab) that binds CTLA4. In some particular embodiments, the NLP-polypeptide conjugate comprises two antigen-binding polypeptides that bind two different HER2 epitopes.

d. Short Peptide Components

In some embodiments, the NLP-polypeptide conjugate comprises (such as further comprises) at least one C₄₋₂₈ fatty-acyl covalently attached to a short peptide (e.g., a CKP or a CKP variant) comprising between 1-80, 10-70, or 20-60 amino acids.

In some embodiments, the C₄₋₂₈ fatty-acyl is a C₁₆ fatty-acyl. In some embodiments, the C₄₋₂₈ fatty-acyl comprises a functional group (e.g., a functional group described elsewhere herein). In some embodiments, the functional group is located directly on, or part of the chemical structure of the hydrocarbon tail of the C₄₋₂₈ fatty-acyl. In some embodiments, the functional group reacts with a corresponding or complementary functional group (e.g., as described elsewhere herein) on one or more amino acid residues of the short peptide. In some embodiments, the corresponding or complementary functional group is located C-terminally on the peptide (e.g., a CKP or a CKP variant). In some embodiments, the corresponding or complementary functional group is located N-terminally on the peptide (e.g., a CKP or a CKP variant). In some embodiments, the C₄₋₂₈ fatty-acyl conjugated to the epsilon amino group of a lysine side chain at the N terminus of the short peptide (e.g., a CKP or a CKP variant). In some embodiments, the conjugate comprises (such as further comprises) between 1-100, 10-90, 20-80, 30-70, 40-60, or 60 short peptides (e.g., 1-100, 10-90, 20-80, 30-70, 40-60, or 60 C₄₋₂₈ fatty-acyl molecules, each covalently attached to a short peptide, e.g., a CKP or a CKP variant). In some embodiments, the molar ratio of short peptide:NLP is between 1-100, 10-90, 20-80, 30-70, 40-60, or 60. In some embodiments, the molar ratio of short peptide:antigen-binding polypeptide in the NLP is between 1-100, 10-90, 20-80, 30-70, or 40-60. In some embodiments, the molar ratio of short peptide:antigen-binding polypeptide of an NLP that comprises both a short peptide and an antigen-binding polypeptide is between about 1:60 and 60:1, including any ratio within this range. In some embodiments, the molar ratio of short peptide:antigen-binding polypeptide of an NLP that comprises both a short peptide and an antigen-binding polypeptide is about 20. In some embodiments, the NLP comprises between 1-50 antigen-binding polypeptides (e.g., Fabs or Fab-like polypeptides) and between 1-100 (such as about 60) short peptides (e.g., CKPs or a CKP variants). In some embodiments, the peptide-NLP-polypeptide conjugate comprises 5-30, 10-25, 15-20, or 18 molecules of a short peptide (e.g., a CKP or a CKP variant), and 5-40, 10-35, 15-30, 20-25, or 23 molecules of an antigen-binding polypeptide (e.g., a Fab or Fab-like molecule). In some embodiments, the peptide-NLP-polypeptide conjugate comprises or 3-30, 5-20, 10-15, or 13 molecules of a short peptide (e.g., a CKP or a CKP variant) and 10-60 molecules of an antigen-binding polypeptide (e.g., Fab or Fab-like molecule). In some embodiments, the peptide-NLP-polypeptide conjugate comprises 20-80, 25-70, 30-60, 35-50, or 40 molecules of a short polypeptide (e.g., a CKP or a CKP variant) and 10-60 molecules of an antigen-binding polypeptide (e.g., Fab or Fab-like molecule). In some embodiments, the molar ratio of short peptide:antigen-binding polypeptide in the NLP is between 1-100, 10-90, 20-80, 30-70, or 40-60. In some embodiments, the molar ratio of short peptide:antigen-binding polypeptide of an NLP that comprises both a short peptide and an antigen-binding polypeptide is between about 1:60 and 60:1, including any ratio within this range. In some embodiments, the molar ratio of short peptide:antigen-binding polypeptide of an NLP that comprises both a short peptide and an antigen-binding polypeptide is about 20.

In some embodiments, incorporation of peptide-conjugated C₄₋₂₈ fatty-acyl into the peptide-NLP-polypeptide conjugate increases the avidity, activity, or potency of the peptide (e.g., the CKP or the CKP variant), e.g., as described above for antigen-binding polypeptides.

In some embodiments, the peptide-NLP-polypeptide conjugate comprises two or more different short peptides (e.g., CKPs or CKP variants) (such as 2, 3, 4, 5, 6, 7, 8, 9, or 10 different short peptides, such as CKPs or CKP variants) that, e.g., demonstrate two or more different activities, e.g., different target binding specificities and/or different therapeutic activities. The peptide-NLP-polypeptide conjugates disclosed herein provide a multispecific platform that can be expanded to multi-specific formats and/or high valencies not only with respect to the antigen-binding polypeptide (as described above), but also with respect to the short peptide (e.g., a CKP or a CKP variant). For example, in some embodiments, the peptide-NLP-polypeptide conjugate comprises 2, 3, 4, 5, 6, 7, or 8 different short peptides (e.g., CKPs or CKP variants), e.g., 2, 3, 4, 5, 6, 7, or 8 different peptides that bind 2, 3, 4, 5, 6, 7, or 8 different antigens or epitopes. In some embodiments, the peptide-NLP-polypeptide conjugate comprises 10-12, 10-15, 10-20, 15-20, 15-25, 15-30, or 20-30 different short peptides (e.g., CKPs or CKP variants), e.g., 10-12, 10-15, 10-20, 15-20, 15-25, 15-30, or 20-30 different peptides that bind 10-12, 10-15, 10-20, 15-20, 15-25, 15-30, or 20-30 different antigens or epitopes. In some embodiments, the peptide-NLP-polypeptide conjugate comprises 25-30, 25-50, 30-45, 30-50, 40-50, 30-60, or 40-60 different short peptides (e.g., CKPs or CKP variants), e.g., 25-30, 25-50, 30-45, 30-50, 40-50, 30-60, or 40-60 different peptides that bind 25-30, 25-50, 30-45, 30-50, 40-50, 30-60, or 40-60 different antigens or epitopes.

In some embodiments, the peptide-NLP-polypeptide conjugate comprises a short peptide (e.g., a CKP or a CKP variant) and an antigen-binding peptide (e.g., a Fab or Fab-like molecule). In some embodiments, the short peptide binds the same target as the antigen-binding polypeptide. In some embodiments, the short peptide binds a different target than the antigen-binding polypeptide. In some embodiments, the short peptide exhibits an activity (e.g., a therapeutic activity) that is complementary to or synergistic with the activity (e.g., therapeutic activity) of the antigen-binding polypeptide. For example, in some embodiments, the peptide-NLP-polypeptide conjugate comprises two short peptides that bind two different antigens, wherein the two different antigens are selected from the following pairs: CD3 and CD19; CD3 and EpCAM; CD3 and CEA; CD16 and CD30; CD16 and CD33; Ang-2 and VEGF-A; and Factor X and Factor IXa. In some embodiments, the conjugate comprises a short peptide (e.g., a CKP or CKP variant) and an antigen-binding polypeptide, wherein the short peptide and the antigen-binding polypeptide bind two different antigens, In some embodiments, the two different antigens are selected from the following pairs: CD3 and CD19; CD3 and EpCAM; CD3 and CEA; CD16 and CD30; CD16 and CD33; Ang-2 and VEGF-A; and Factor X and Factor IXa.

In some embodiments, a conjugate comprises a short peptide (e.g., a CKP or a CKP variant) that binds a target selected from OX40, DR4, GITR, Tie2, factor D, VEGF, MerTK, CD3, and Lymphotoxin beta receptor. In some embodiments, the conjugate comprises at least two different short peptides (e.g., CKPs or CKP variants), or a short peptide (e.g., a CKP or a CKP variant) and an antigen-binding peptide (e.g., a Fab or Fab-like molecule), that bind any combination of the preceding targets. In some embodiments, a conjugate comprises a short peptide (e.g., a CKP or CKP variant) that binds a target selected from factor D, VEGF, Tie2, and DR4. In some embodiments, the conjugate comprises at least two different short peptides (e.g., CKPs or CKP variants), or a short peptide (e.g., a CKP or a CKP variant) and an antigen-binding peptide (e.g., a Fab or Fab-like molecule), that bind any combination of the preceding targets.

In some embodiments, the short peptide is a cysteine-knot peptide (CKP). Cystine-knot peptides are typically between 20 and 60 amino acids and length and comprise six conserved cysteines that form three intramolecular disulfide bonds arranged in a knotted conformation known as the cystine-knot motif, i.e., a ring formed by two disulfide bonds that is threaded by the third disulfide bond (Craik et al. (2001) Toxicon 39(1): 43-60). Such structural motif, which is present in peptides and proteins from a variety of species, confers high stability to the peptide framework against thermal, chemical and enzymatic degradation (Colgrave et al. (2004) Biochemistry 43, 5965-5975). Exemplary CKPs include, but are not limited to, e.g., EETI-II (UniProt Accession No. P12071), cyclopsychotride A (UniProt Accession No. P56872), cycloviolacin O1 (Uniprot Accession No. P82230), cycloviolacin O12 (UniProt Accession No. P83836), a kalata (e.g., kalata B1 (Uniprot Accession No. P56254), kalata B8 (Uniprot Accession No. P85175), etc.), palicourein (Uniprot Accession No. P84645), tricyclon A (Uniprot Accession No. B6E617), a Momordica cochinchinensis trypsin inhibitor, e.g., MCoTI 1 (Uniprot Accession No. P82408), a Spinacia oleracea trypsin inhibitor, e.g., SOTI 1 (Uniprot Accession No. P84779), circulin A (Uniprot Accession No. P56871), circulin B (Uniprot Accession No. 56879), cyclopsychotride (Uniprot Accession No. P56872), and varv peptide A (Uniprot Accession No. P58446). In some embodiments, the CKP is EETI-II. The amino acids sequence of EETI-II is GCPRILMRCKQDSDCLAGCVCGPNGFCG (SEQ ID NO: 1).

The high amino acid sequence diversity present in the loop regions flanking the conserved cysteine residues in CKPs suggests that non-native sequences might be tolerated in these regions. CKP backbones have been used as frameworks for peptide grafting and affinity maturation to generate highly stable peptides with structures and/or biological activities that are significantly different from native CKPs. For Example, the Ecballium elaterium trypsin inhibitor II (EETI-II) has been used as a molecular scaffold by rationally substituting this trypsin binding loop (PRILMR) with grafted biologically-active peptides against targets such as elastase, thrombopoietin, and integrins. See, e.g., Kimura et al. (2009) Proteins 77, 359-369; Gunasekera et al. (2008) J. Med. Chem. 51: 7697-7704; Krause et al. (2007) Proteins 77, 359-369; Hilpert et al. (2003) J. Biol. Chem. 278, 24986-24993; and Kimura et al. (2011) PLoS One 6, e16112). Additionally, peptide derivatives of EETI-II that bind targets of interest, e.g., VEGFA and LRP6, have been generated via affinity maturation. See WO 2017/049009. In some embodiments, the peptide is a CKP variant. In some embodiments, the CKP variant comprises one or more amino acid insertions, deletions, and/or substitutions in one or more loop sequences relative to the corresponding one or more loop sequences of a wild type CKP. Additionally or alternatively, in some embodiments, the CKP variant comprises one or more amino acid insertions, deletions, and/or substitutions at the N terminus relative to a wild type CKP. Additionally or alternatively, in some embodiments, the CKP variant comprises one or more amino acid insertions, deletions, and/or substitutions at the C-terminus relative to a wild type CKP. Additionally or alternatively, in some embodiments, the CKP variant comprises a chemical modification at the N-terminus relative to a wild type CKP. Additionally or alternatively, in some embodiments, the CKP variant comprises a chemical modification at the C-terminus relative to a wild type CKP. In some embodiments, the CKP variant comprises a modification that permits the covalent conjugation of a C₄₋₂₈ fatty-acyl (e.g., C₁₆ fatty-acyl) to the CKP variant (e.g., at or near the N-terminus of the CKP variant, or at or near the C-terminus of the CKP variant). In some embodiments, the CKP variant further comprises (e.g., has been modified to further comprise) an additional lysine residue its N-terminus relative to the wild-type CKP from which it was derived. In some embodiments, the CKP variant binds to a target that is different from the target bound by the wild type CKP from which it is derived. In some embodiments, the CKP variant demonstrates an activity (e.g., a therapeutic activity) that is different from the target bound by the wild type CKP from which it is derived. In some embodiments, the CKP variant is derived from EETI-TT (SEQ ID NO: 1).

In some embodiments, the activity of the short peptide in a conjugate (e.g., peptide-NLP-polypeptide conjugate) comprising an antigen-binding polypeptide (e.g., Fab or Fab-like molecule) and the short peptide is higher, e.g., about any one of 2-, 3-, 4-, 5-, 6-, 7-, 8-, 9-, or 10-fold higher, than the activity of the short peptide in a conjugate (e.g., peptide-NLP conjugate) comprising the short peptide, i.e., without the antigen-binding polypeptide. In some embodiments, the activity of the short peptide is between about 2-fold to 20-fold higher, e.g., about 5-fold higher. In some embodiments, the short peptide is a CKP, e.g., EETI-II. In some embodiments, the short peptide is a CKP variant that is derived from EETI-II.

### III. Nanolipoprotein Conjugates Comprising a Short Peptide

Another aspect of the invention relates to nanolipoprotein particle conjugates comprising at least one short peptide comprising between 1-80, 10-70, or 20-60 amino acids (i.e., an NLP-incorporated peptide other than a scaffold protein). As discussed elsewhere herein, “nanolipoprotein particles” or NLPs refer to supramolecular complexes comprising lipid and scaffold proteins, in particular, comprising membrane-forming lipids and scaffold proteins, such as apolipoproteins. NLPs generally are formed through a self-assembly process, where the membrane-forming lipids form a lipid bilayer having a discoidal shape (see reference 19 below Example 5), and where the hydrophobic periphery of the disc is stabilized by binding to the scaffold protein, which encircles the bilayer disc. In some embodiments, the membrane forming lipids comprise a C₄₋₂₈ fatty-acyl (e.g., a C₁₆ fatty-acyl). In some embodiments, the membrane forming lipids comprise an alkylphosphocholine, ether lipid, glycolipid, lysosphingolipid, lysoglycerophospholipids, phospholipid, sphingolipid, and/or sterol. Additionally or alternatively, in some embodiments, the membrane-forming lipid is a phospholipid or a combination of different phospholipids. Exemplary phospholipids include, but are not limited to, e.g., dimyristoylphosphatidylcholine (DMPC), dioleoylphospho-ethanolamine (DOPE), dioleoylphosphatidylcholine (DOPC), dioleoylphosphoserine (DOPS), and dipalmitoyl-phosphatidyl-choline (DPPC), and combinations thereof. In a particular embodiment, the membrane-forming lipid is DOPE and/or DOPC. In some embodiments, the membrane-forming lipids are non-lipid amphipathic molecules, such as digylcerol tetraethers, cholesterol, ergosterol, and the like.

In some embodiments, the membrane-forming lipid is a biological molecule, i.e. a molecule produced by a living organism, e.g., bacteria, yeast, or a mammal.

In such peptide-NLP conjugates, the bilayer formed by membrane-forming lipids is stabilized by one or more scaffold proteins, i.e., a scaffold protein descried elsewhere herein. In some embodiments, scaffold protein is an apolipoprotein. In some embodiments, the scaffold protein is apolipoprotein A, apolipoprotein B, apolipoprotein C, apolipoprotein D, apolipoprotein H, apolipoprotein E, or a combination of any one or more of the preceding. In some embodiments, the scaffold protein is truncated version of any of the preceding apolipoproteins that is capable of stabilizing said bilayer. In some embodiments, the scaffold protein is a truncated version of apoE3 (e.g. apoE322k), a truncated version of apoE2 (e.g. apoE222k), or a truncated version of apoA1 (e.g. Δ49ApoA1, MSP1, MSP1T2, MSP1E3D1). In some embodiments, the scaffold protein and membrane-forming lipid components are both biological molecules produced by a living organism, and form NLPs free of non-biologically derived materials.

One of skill will understand that the scaffold protein and membrane-forming lipids can be provided in suitable molar ratios to facilitate peptide-NLP conjugate assembly. In some embodiments, the scaffold protein and membrane-forming lipids are in a molar ratio of from 1:50 to 1:100; from 1:60 to 1:90; or from 1:70 to 1:80. In some embodiments, the scaffold protein and membrane-forming lipids are in a molar ratio of 1:60, 1:70, 1:75, 1:80, 1:85, 1:90, or 1:100. In a specific embodiment, the scaffold protein and membrane-forming lipids are in a molar ratio of 1:80. In some embodiments, a ratio is used such that most or all of the membrane-forming lipids arrange as bilayers, and none or few remain unassembled.

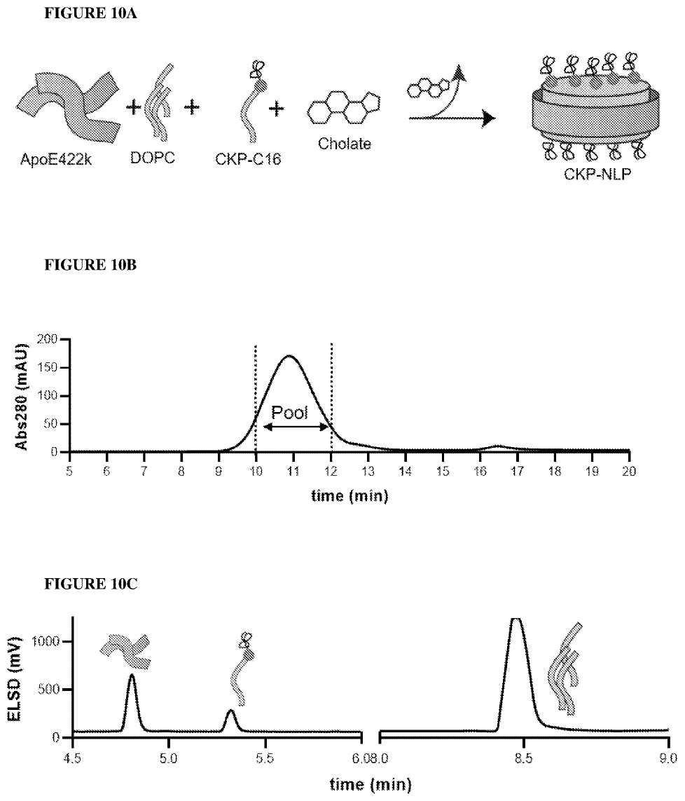

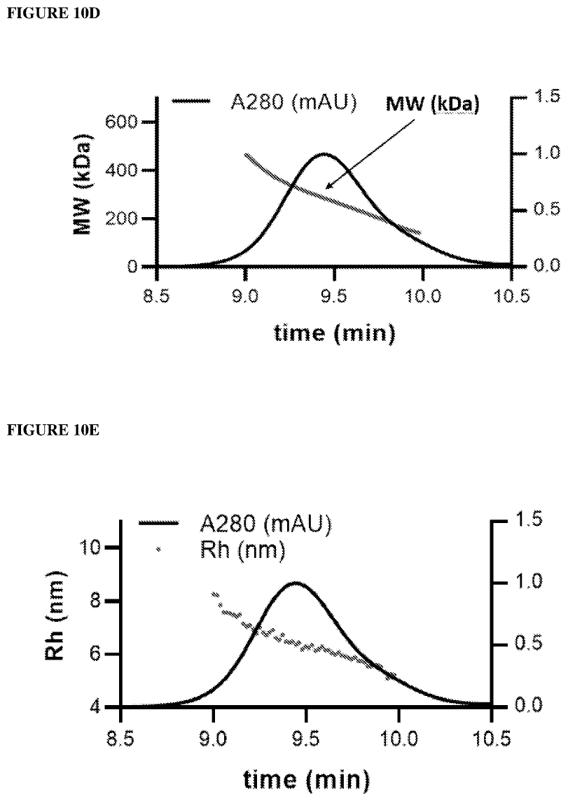

In some embodiments, the peptide-NLP conjugate comprises at least one C₄₋₂₈ fatty-acyl membrane forming lipid covalently attached to a short peptide comprising between 1-80, 10-70, or 20-60 amino acids. In some embodiments, the C₄₋₂₈ fatty-acyl is a C₁₆ fatty-acyl. In some embodiments, the C₄₋₂₈ fatty-acyl comprises a functional group (e.g., a functional group described elsewhere herein). In some embodiments, the functional group is located directly on, or part of the chemical structure of the hydrocarbon tail of the C₄₋₂₈ fatty-acyl. In some embodiments, the functional group reacts with a corresponding or complementary functional group (e.g., as described elsewhere herein) on one or more amino acid residues of the short peptide. In some embodiments, the corresponding or complementary functional group is located C-terminally on the peptide. In some embodiments, the corresponding or complementary functional group is located N-terminally on the peptide. In some embodiments, the C₄₋₂₈ fatty-acyl conjugated to the epsilon amino group of a lysine side chain at the N terminus of the short peptide. In some embodiments, the peptide-NLP conjugate comprises between 1-100, 10-90, 20-80, 30-70, 40-60, or 60 short peptides (e.g., 1-100, 10-90, 20-80, 30-70, 40-60, or 60 C₄₋₂₈ fatty-acyl (e.g., C₁₆ fatty-acyl) membrane forming lipids covalently attached to a short peptide). In some embodiments, the molar ratio of short peptide:NLP is between 1-100, 10-90, 20-80, 30-70, 40-60, or 60. Successful incorporation of a short peptide into an NLP, as described herein, can be confirmed and/or quantitated using techniques described in Examples 6 and 7 below, e.g., liquid chromatography-mass spectrometry, gel electrophoresis, high-performance liquid chromatography (HPLC), and nuclear magnetic resonance (NMR). FIG. 10A depicts a schematic of the assembly of an exemplary NLP comprising a short peptide.

In some embodiments, incorporation of peptide-conjugated C₄₋₂₈ fatty-acyl membrane forming lipids facilitates multimerization of the short peptide, e.g., where the conjugates provides two or more molecules of the short peptide, that is, where multiple copies of the same peptide are incorporated into the peptide-NLP conjugate. In this manner, the avidity, activity, or potency of the peptide is increased, e.g., as described elsewhere herein for the antigen-binding polypeptide.

In some embodiments, the peptide-NLP conjugate comprises two or more different short peptides (such as 2, 3, 4, 5, 6, 7, 8, 9, or 10 different short peptides) that, e.g., demonstrate two or more different activities, e.g., different target binding specificities and/or different therapeutic activities. The peptide-NLP conjugates disclosed herein provide a multispecific platform that can be expanded to multi-specific formats and/or high valencies with respect to the short peptide. For example, in some embodiments, the peptide-NLP conjugate comprises 2, 3, 4, 5, 6, 7, or 8 different short peptides, e.g., 2, 3, 4, 5, 6, 7, or 8 different peptides that bind 2, 3, 4, 5, 6, 7, or 8 different antigens or epitopes. In some embodiments, the peptide-NLP conjugate comprises 10-12, 10-15, 10-20, 15-20, 15-25, 15-30, or 20-30 different short peptides, e.g., 10-12, 10-15, 10-20, 15-20, 15-25, 15-30, or 20-30 different peptides that bind 10-12, 10-15, 10-20, 15-20, 15-25, 15-30, or 20-30 different antigens or epitopes. In some embodiments, the peptide-NLP conjugate comprises 25-30, 25-50, 30-45, 30-50, 40-50, 30-60, or 40-60 different short peptides, e.g., 25-30, 25-50, 30-45, 30-50, 40-50, 30-60, or 40-60 different peptides that bind 25-30, 25-50, 30-45, 30-50, 40-50, 30-60, or 40-60 different antigens or epitopes.

Configurations for providing specific multi-valencies and multi-specificities with respect to the short peptide will be apparent to those of skill in the art. For example, in some embodiments, the peptide-NLP conjugate comprises two short peptides that bind two different antigens, wherein the two different antigens are selected from the following pairs: CD3 and CD19; CD3 and EpCAM; CD3 and CEA; CD16 and CD30; CD16 and CD33; Ang-2 and VEGF-A; and Factor X and Factor IXa.

In some embodiments, a peptide-NLP conjugate comprises a short peptide that binds a target selected from OX40, DR4, GITR, Tie2, factor D, VEGF, MerTK, CD3, and Lymphotoxin beta receptor. In some embodiments, the peptide-NLP conjugate comprises at least two different short peptides that bind any combination of the preceding targets. In some embodiments, a peptide-NLP conjugate comprises a short peptide that binds a target selected from factor D, VEGF, Tie2, and DR4. In some embodiments, the peptide-NLP conjugate comprises at least two different short peptides that bind any combination of the preceding targets.

In some embodiments, the short peptide is a cysteine-knot peptide (CKP). Exemplary CKPs include, but are not limited to, e.g., EETI-II (UniProt Accession No. P12071), cyclopsychotride A (UniProt Accession No. P56872), cycloviolacin 01 (Uniprot Accession No. P82230), cycloviolacin 012 (UniProt Accession No. P83836), a kalata (e.g., kalata B1 (Uniprot Accession No. P56254), kalata 88 (Uniprot Accession No. P85175), etc.), palicourein (Uniprot Accession No. P84645), tricyclon A (Uniprot Accession No. B6E617), a Momordica cochinchinensis trypsin inhibitor, e.g., MCoTI 1 (Uniprot Accession No. P82408), a Spinacia oleracea trypsin inhibitor, e.g., SOTI 1 (Uniprot Accession No. P84779), circulin A (Uniprot Accession No. P56871), circulin B (Uniprot Accession No. 56879), cyclopsychotride (Uniprot Accession No. P56872), and varv peptide A (Uniprot Accession No. P58446). In some embodiments, the CKP is EETI-II. The amino acids sequence of EETI-II is GCPRILMRCKQDSDCLAGCVCGPNGFCG (SEQ ID NO: 1).

In some embodiments, the peptide is a CKP variant. In some embodiments, the CKP variant comprises one or more amino acid insertions, deletions, and/or substitutions in one or more loop sequences relative to the corresponding one or more loop sequences of a wild type CKP.

Additionally or alternatively, in some embodiments, the CKP variant comprises one or more amino acid insertions, deletions, and/or substitutions at the N terminus relative to a wild type CKP. Additionally or alternatively, in some embodiments, the CKP variant comprises one or more amino acid insertions, deletions, and/or substitutions at the C-terminus relative to a wild type CKP. Additionally or alternatively, in some embodiments, the CKP variant comprises a chemical modification at the N-terminus relative to a wild type CKP. Additionally or alternatively, in some embodiments, the CKP variant comprises a chemical modification at the C-terminus relative to a wild type CKP. In some embodiments, the CKP variant comprises a modification that permits the covalent conjugation of a C₄₋₂₈ fatty-acyl (e.g., C₁₆ fatty-acyl) to the CKP variant (e.g., at or near the N-terminus of the CKP variant, or at or near the C-terminus of the CKP variant). In some embodiments, the CKP variant binds to a target that is different from the target bound by the wild type CKP from which it is derived. In some embodiments, the CKP variant demonstrates an activity (e.g., a therapeutic activity) that is different from the target bound by the wild type CKP from which it is derived. In some embodiments, the CKP variant is derived from EETI-TT (SEQ ID NO: 1).

In some embodiments, the activity (e.g., biological activity) of a short peptide in a peptide-NLP conjugate (e.g., NLP conjugate) comprising the short peptide is lower, e.g., about any one of 2-, 3-, 4-, 5-, 6-, 7-, 8-, 9-, or 10-fold lower, than the activity of the free short peptide, i.e., peptide that has not been incorporated into the peptide-NLP conjugate. In some embodiments, the activity of the short peptide is about 5-fold lower. In some embodiments, the activity (e.g., biological activity) of a short peptide in a peptide-NLP conjugate comprising the short peptide is 80-90%, 90-95%, or 95-99% of the activity of the free peptide. In some embodiments, the short peptide is a CKP, e.g., EETI-II. In some embodiments, the short peptide is a CKP variant that is derived from EETI-JI.

Methods of increasing the stability (e.g., in vitro stability or in vivo half-life) of NLPs are known in the art and can be used to increase the stability of peptide-NKP conjugates. See, e.g., Gilmore S F, Carpenter T S, Ingolfsson H I, Peters S K G, Henderson P T, Blanchette C D, Fischer NO “Lipid composition dictates serum stability of reconstituted high-density lipoproteins: implications for in vivo applications” Nanoscale, 2018, 10 (16):7420-7430; and Gilmore S F, Blanchette C D, Scharadin T M, Hura G L, Rasley A, Corzett M, Pan C X, Fischer N O, Henderson P T. “Lipid Cross-Linking of Nanolipoprotein Particles Substantially Enhances Serum Stability and Cellular Uptake” ACS Applied Material Interfaces, 2016, 8:32.

The conjugates of the present invention, e.g., peptide-NLP conjugates, peptide-NLP-polypeptide conjugates, and NLP-polypeptide conjugates, may be administered to a subject in need thereof on their own or as part of a pharmaceutical composition. Such pharmaceutical compositions are discussed below.

### IV. Pharmaceutical Compositions and Administration

In another aspect, the present invention provides pharmaceutical compositions, e.g., a pharmaceutical composition comprising at least one conjugate (e.g., a peptide-NLP conjugate, a peptide-NLP-polypeptide conjugate, and/or an NLP-polypeptide conjugate) of the invention and a pharmaceutically acceptable vehicle. A pharmaceutical composition may also be referred to as a “medicament” or as a “formulation.” Conjugates (e.g., peptide-NLP conjugates, peptide-NLP-polypeptide conjugates, and NLP-polypeptide conjugates) of the present invention, particularly an NLP-Fab conjugate or a peptide-NLP-4ab conjugate, can be prepared in a physiologically acceptable formulation comprising a pharmaceutically acceptable vehicle based on known techniques. For example, the conjugate is combined with a pharmaceutically acceptable vehicle or carrier to form a pharmaceutical composition.

A “pharmaceutically acceptable vehicle” or “pharmaceutically acceptable carrier” refers to an ingredient in a pharmaceutical composition, other than an active ingredient, which is nontoxic to a subject, and that does not interact in a deleterious way with other ingredients of the composition. Generally, the pharmaceutically acceptable vehicle is incorporated into a pharmaceutical composition and administered to a subject without causing undesirable biological effects that outweigh intended beneficial effects of the active ingredient(s). Pharmaceutically acceptable vehicles include, but are not limited to, buffers, solvents, excipients, stabilizers, diluents, preservatives, and the like. The vehicle used typically depends on the form of the pharmaceutical composition and/or the intended route of administration. Pharmaceutically acceptable carriers or excipients have preferably met the required standards of toxicological and manufacturing testing and/or are included on the Inactive Ingredient Guide prepared by the U.S. Food and Drug Administration.

The pharmaceutical composition may be administered to a subject by intravenous administration, e.g., as a bolus, or by continuous infusion over a period of time. The pharmaceutical composition may be administered by intravenous, intradermic, intramuscular, intraperitoneal, intracerobrospinal, intra-articular, intrasynovial, or subcutaneous administration. In some embodiments, the pharmaceutical compositions is administered by intratracheal vaginal, oral, sublingual, or ocular route of administration. In some embodiments, the pharmaceutical compositions is administered to the brain or central nervous system.

The compositions of the present invention may be administered to a subject in the form of a solid, liquid or aerosol at a suitable dose. Examples of solid compositions include pills, creams, and implantable dosage units. Pills may be administered orally, Therapeutic creams may be administered topically. Implantable dosage units may be administered locally, for example, at a tumor site, or may be implanted for systematic release of the pharmaceutical composition, for example, subcutaneously. Examples of liquid compositions include formulations adapted for injection intramuscularly, subcutaneously, intravenously, intra-arterially, and formulations for topical and intraocular administration. Examples of aerosol formulations include inhaler formulations for administration to the lungs.

In certain embodiments, the pharmaceutical composition is administered systemically. The pharmaceutical composition may be administered parenterally or non-parenterally. For parenteral administration, the pharmaceutical composition will generally comprise sterile aqueous or non-aqueous solutions, suspensions and emulsions, In some embodiments, the solution or suspension may be prepared at the time of use, e.g., by dissolving a powdered composition, e.g., provided in lyophilized form, in a suitable aqueous liquid (e.g., distilled water) or non-aqueous solvent. Non-aqueous solvents include without being limited to it, propylene glycol, polyethylene glycol, vegetable oil such as olive oil, and injectable organic esters such as ethyl oleate. Aqueous solvents may be chosen from water, alcohol/aqueous solutions, emulsions, or suspensions, including saline and buffered media. Parenteral vehicles include sodium chloride solution, Ringer's dextrose, dextrose and sodium chloride, lactated Ringer's, or fixed oils. Preservatives may also be present such as, for example, antimicrobials, anti-oxidants, chelating agents, inert gases, etc.

In some embodiments, the pharmaceutical composition is administered to the brain or central nervous system, e.g., in the treatment of neurological disease. In particular embodiments, the conjugate (e.g., a NLP-Fab conjugate, a NLP-Fab-like molecule conjugate, a peptide-NIP-Fah conjugate, or a peptide-NLP-Fab-like molecule conjugate) can cross the blood-brain barrier, allowing delivery. e.g. targeted delivery, to the brain (brain parenchyma) or central nervous system following administration either locally or systematically. In specific embodiments for use in delivery to the brain or central nervous system, the scaffold protein is an apolipoprotein, such as apolipoprotein E, in particular, apoE2 and/or apoE4, and the conjugate shows biodistribution to the brain parenchyma following systemic, e.g., intravenous, delivery.

a. Subcutaneous and Ocular Preparations

In some embodiments, the present invention provides conjugates of the invention in highly concentrated but low viscosity formulations, as described in more detail below. Such formulations are advantageous, e.g., where the route of administration limits the volume of formulation that can be delivered, such as in the case of subcutaneous and ocular routes of administration.

Pharmaceutical compositions for subcutaneous delivery generally include excipients comprising saccharide stabilizers (e.g. sucrose or trehalose) and/or surfactants (e.g. polysorbate 20 or polysorbate 80) and/or amino acids (e.g. histidine, arginine, glycine, and/or alanine). For subcutaneous delivery, the formulation may be delivered via syringe (e.g. pre-filled syringe); autoinjector; injection device (e.g. the INJECT-EASE™ and GENJECT™ device); injector pen (such as the GENPEN™); needleless devices (e.g. MediJector™ and BioJector™); subcutaneous patch delivery systems, or other device suitable for administering a solution or suspension formulation subcutaneously.

For ocular delivery, the pharmaceutical composition may be formulated to contain, buffers, stabilizers, preservatives and/or bulking agents, to render the composition suitable for ocular administration to a patient. Pharmaceutical compositions for ocular delivery generally comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues polypeptides; proteins, such as serum albumin, gelatin, or immunoglobulins; hydrophilic polymers such as polyvinylpyrrolidone: amino acids such as glycine, glutamine, asparagine, histidine, arginine, or lysine; monosaccharides, disaccharides, and other carbohydrates including glucose, mannose, or dextrins: chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Sustained-release preparations may be prepared. Suitable examples of sustained-release preparations include sustained-recast matrices of solid hydrophobic polymers containing the conjugate, which matrices arm in the form of shaped articles, e.g. films, or microcapsules. A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. Non-limiting examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

In certain embodiments, the pharmaceutical composition comprises one or more permeability enhancers that permit a conjugate of the present invention to penetrate the cornea. Examples of such permeability enhancers include, e.g., surfactants, bile acids, chelating agents, preservatives, cyclodextrins (i.e., cylindrical oligonucleotides with a hydrophilic outer surface and a lipophilic inner surface that form complexes with lipophilic drugs), etc. Such permeability enhancers increase chemical stability and bioavailability and decrease local irritation. In certain embodiments, a pharmaceutical composition provided herein additionally comprises agents that increase the absorption and distribution of a conjugate of the invention in various ocular compartments.

In certain embodiments, a pharmaceutical composition provided herein is formulated as an in-situ gelling system, e.g., a viscous polymer-based liquid that exhibits sol-to-gel phase transition on the ocular surface due to change in a specific physicochemical parameter (ionic strength, temperature, pH, or solvent exchange) when the composition comes into contact with tear fluid. In certain embodiments, a pharmaceutical composition provided herein is formulated as an eye spray. In certain embodiments, a pharmaceutical composition provided herein is formulated as a microemulsion. Further details regarding various ophthalmic pharmaceutical formulations are provided in, e.g., Gaikwad et al. (2013) Indo Amer J Pharm Res. 3, 3216-3232; Achouri et al. (2012) Drug Dev Indust Pharm. 39, 1599-1617; Lu (2010) Recent Pat Drug Deliv Formul. 4, 49-57; Baranowski et al. (2014) Sci World J. doi.org/10.1155/2014/861904; Lang (1995) Adv Drug Deliv Rev. 16, 39-43; Short (2008) Toxicologic Path. 36, 49-62; and others.

For ocular delivery, the conjugate may be administered by injection, e.g., subconjunctival injection, intracorneal injection, or intravitreal injection; or is administered topically, e.g. in the form of eye drops; or by an intravitreal device that continuously delivers the conjugates into the eye.

b. Dosages Generally, the pharmaceutical composition will include an effective amount of an NLP conjugate described herein. An “effective amount” of an agent (e.g., an antigen-binding polypeptide or other drug) refers to the amount necessary to result in a physiological change in the cell or tissue to which the agent is administered, typically to produce a beneficial effect for the subject receiving the pharmaceutical composition.

The dosage of the pharmaceutical composition will depend on various factors such as, e.g. the condition of being treated, the severity and course of the condition, whether the conjugate is administered for preventive or therapeutic purposes, other clinical factors such as weight, size, sex and general health condition of the subject, the particular NLP conjugate to be administered, other drugs being administered concurrently, the route of administration, previous therapy, the subject's clinical history, and the discretion of the attending physician.

Generally, the regimen for regular administration of the pharmaceutical composition should be in the range of 1 μg to 5 g units per day. Progress can be monitored by periodic assessment. The conjugate may be suitably administered at one time or over a series of treatments and may be administered to the subject at any time from diagnosis onwards. The conjugate may be administered as the sole treatment or in combination with other therapeutic agents or therapy approaches useful in treating the condition in question.

c. Low Viscosity Formulations

Another aspect of the invention relates to low viscosity liquid formulations, comprising one or more conjugates described herein. “Viscosity” refers to the measure of resistance of a fluid being deformed by either shear stress or tensile stress; it can be evaluated using a viscometer or rheometer. Unless indicated otherwise, the viscosity measurement (centipoise, cP) is performed at 25° C.

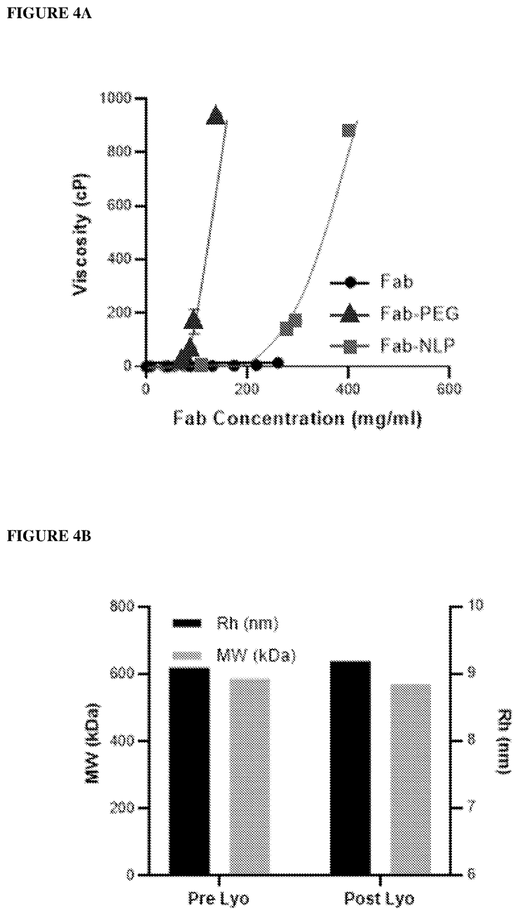

Surprisingly, in some embodiments, conjugates (e.g., NLP-polypeptide conjugates and/or peptide-NLP-polypeptide conjugates) described herein can be provided in high concentration liquid formulations that maintain relatively low viscosity. Typically, at high protein concentrations, protein-protein interactions lead to increases in viscosity. In contrast, the present NLP conjugates (e.g., NLP-polypeptide conjugates and/or peptide-NLP-polypeptide conjugates) allow unexpectedly high protein concentrations before there is a substantial increase in viscosity (see, e.g., Example 3, FIG. 4A, where the impact of Fab loading on the concentration/viscosity relationship was evaluated). This relationship is an important Chemistry, Manufacturing and Controls (CMC) consideration and, in certain embodiments, NLP conjugates of the present invention can be provided in unexpectedly high concentrations in low viscosity liquid formulations. Indeed, significantly higher Fab concentrations were achieved with the Fab-NLP conjugates relative to an alternative multivalent format (Fab-PEG conjugates).

A “low viscosity formulation” (or “low viscosity liquid formulation”) typically has a viscosity below 50 cP, e.g., 40-50 cP; or below 40 cP, e.g., 30-40 cP; or below 30 cP, e.g., 20-30 cP; or below 20 cP, e.g., 10-20 cP. In some embodiments, the viscosity is 10-50 cP, 15-45 cP, 20-40 cP, 25-35 cP, or 30 cP. In some embodiments, the low viscosity formulation has a concentration of over 50 mg of conjugate/mL, e.g., 50-75 mg/mL; over 75 mg of conjugate/mL, e.g., 75-100 mg/mL; over 100 mg of conjugate/mL, e.g., 100-150 mg/mL; over 150 mg of conjugate/mL, e.g., 150-200 mg/mL; over 200 mg of conjugate/mL, e.g., 200-250 mg/mL; over 250 mg of conjugate/mL, e.g., 250-300 mg/mL; over 300 mg of conjugate/mL, e.g., 300-350 mg/mL: over 350 mg of conjugate/mL, e.g., 350-400 mg/mL; or over 400 mg of conjugate/mL, e.g., 400-450 mg/mL. In some embodiments, the concentration of the low viscosity formulation is 100-200, 200-300, or 100-300 mg of conjugate/mL. In some embodiments, the conjugate is an NLP-polypeptide conjugate or a peptide-NLP-polypeptide conjugate. In particular embodiments, the antigen-binding polypeptide of the low viscosity formulation is a Fab, or Fab-like molecule.

In some embodiments, the low viscosity formulation comprises a conjugate having 20-40, 25-35, 20-30, 25-40, 25-30, or 30 antigen-binding polypeptides (e.g., Fabs) per conjugate. In certain embodiments, the antigen-binding polypeptide is a Fab or Fab-like molecule. In certain embodiments, the conjugate comprises 25, 27, 28, 29, 30, 31, 32, 33, 34, or 35 molecules of the antigen-binding polypeptide, e.g., a Fab or Fab-like molecule, per conjugate. In some embodiments, conjugate in the low viscosity formulation further comprises between 1-100, 10-90, 20-80, 30-70, 40-60, or 60 short peptides. Exemplary short peptides are discussed in further detail elsewhere herein.

One of skill in the art will recognize that providing high concentrations of an antigen-binding polypeptide in a low viscosity formulation facilitates delivery of effective amounts of the antigen-binding polypeptide via routes where the volume that can be administered is restrictive. For example, for ocular delivery, injection volume typically is limited to 100 μl (58); for subcutaneous delivery, injection volume typically is limited to 1-2 ml (59, 60). Accordingly, conjugates (e.g., NLP-polypeptide conjugates and/or peptide-NLP-polypeptide conjugates) of the present invention (e.g., find use in high concentration, low viscosity formulations for administration via volume-restricted routes, such as via ocular delivery and subcutaneous administration. Accordingly, in some embodiments, the present invention provides subcutaneous and ocular formulations, that is, pharmaceutical compositions of the invention for administration subcutaneously or via ocular delivery, respectively, to a subject in need thereof.

The subcutaneous or ocular formulations advantageously comprise a high concentration of conjugate (e.g., NLP-polypeptide conjugate and/or peptide-NLP-polypeptide conjugate) in a low viscosity liquid, in accordance with the low viscosity formulations described herein. All of the references cited in this paragraph can be found below Example 5.

d. Lyophilized Formulations

In some embodiments, the pharmaceutical compositions, formulations, and any one of the conjugates of the invention can be lyophilized, and reconstituted. “Lyophilization” refers to a freeze-drying process that helps preserve a perishable material and/or makes it easier to transport. Freeze-drying involves freezing a material and then reducing surrounding pressure while increasing the temperature to allow frozen water in the material to sublime directly into the gas phase. Generally, lyophilization results in a product in which the moisture content thereof is less than 5%, e.g., 3-5%, or less than 3%, e.g., 2-3%, or less than 2%, e.g., 1-2%. A lyophilized product can be subsequently reconstituted prior to use, often in the same containers or vials in which it was lyophilized and stored. Typically, the product is reconstituted (rehydrated) by adding an aqueous liquid, e.g., distilled water or aqueous buffer. Moreover, stability of a therapeutic agent during lyophilization, and its ability to retain activity following reconstitution, are important Chemistry, Manufacturing and Controls (CMC) considerations. See, e.g., Bjelosevic et al. “Excipients in Freeze-Dried Biopharmaceuticals: Contributions Toward Formulation Stability and Lyophilisation Cycle Optimisation” Int J Pharm 2020 Feb. 25; 576.

Surprisingly, in some embodiments, the NLP conjugate (e.g., an NLP-antigen-binding polypeptide conjugate and/or a peptide-NLP-antigen-binding polypeptide conjugate) can be lyophilized and reconstituted without loss of antigen-binding activity (see, e.g., Example 4, FIG. 4B and FIGS. 5A-5D, where Fab-NLP stability upon lyophilization was evaluated as an assessment of the manufacturability of the Fab-NLP). In some embodiments, at least 80%, e.g., 80-90%, at least 90%, e.g., 90-95%, or at least 95%, e.g., 95-99% of antigen-binding activity is retained following lyophilization and reconstitution. In some embodiments, 98%, 99%, or 100% of antigen-binding activity is retained.

For lyophilization, the conjugate (e.g., an NLP-antigen-binding polypeptide conjugate and/or a peptide-NLP-antigen-binding polypeptide conjugate) is provided in a pre-lyophilized formulation that typically is a pH-buffered solution comprising the conjugate. The pH may be from 4-8, or from 5-7. Exemplary buffers include histidine, phosphate, Tris, citrate, succinate and other organic acids. In some embodiments, a lyoprotectant is added to the pre-lyophilized formulation. Non-limited examples of lyoprotectants include non-reducing sugars, such as sucrose or trehalose. In particular embodiments, trehalose is added to the solution containing a conjugate of the invention to be lyophilized. The amount of lyoprotectant in the pre-lyophilized formulation is generally such that, upon reconstitution, the resulting formulation will be isotonic. However, hypertonic reconstituted formulations may also be suitable.

In some embodiments, lyophilization occurs in the presence of trehalose, e.g., where the conjugate is in the presence of 40-200 mM trehalose. In particular embodiments, trehalose is used at concentrations of 50-150 mM, 60-100 mM, or 70-90 mM during lyophylization of a conjugate of the invention, e.g., a conjugate comprising a Fab as the antigen-binding polypeptide. In some embodiments, lyophilization occurs in the presence of 60 mM, 65 mM, 70 mM, 75 mM, 80 mM, 85 mM, 90 mM, 95 mM, or 100 mM trehalose, e.g., where the conjugate comprises a Fab as the antigen-binding polypeptide. In some embodiments, lyophilization occurs in the presence of 80 mM trehalose.

In some embodiments, lyophilization occurs at a concentration of 1-10 mg conjugate/mL. In particular embodiments, the conjugate comprises a Fab as the antigen-binding, and the conjugate is lyophilized at 2-8 mg conjugate/mL, 3-6 mg conjugate/mL, or 5 mg conjugate/mL. In some embodiments, the conjugate comprises a Fab, and lyophilization occurs in the presence of 70 mM, 75 mM, 80 mM, 85 mM, or 90 mM trehalose; and at 2-8 mg conjugate/mL, 3-6 mg conjugate/mL, or 5 mg conjugate/mL. In a particular embodiment, the conjugate comprises a Fab, and lyophilization occurs in the presence of 80 mM trehalose, and at 5 mg conjugate/mL.

In some embodiments, it has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68 etc.). The amount of surfactant added is such that it reduces aggregation of the reconstituted conjugate and minimizes the formation of particulates after reconstitution. For example, the surfactant may be present in the pre-lyophilized formulation in an amount from 0.001-0.5%, and preferably from 0.005-0.05%.

In certain embodiments, a mixture of the lyoprotectant (e.g., trehalose) and a bulking agent (e.g. mannitol or glycine) is used in the preparation of the pre-lyophilization formulation. The bulking agent may allow for the production of a uniform lyophilized cake without excessive pockets therein etc.

After the conjugate, buffer, lyoprotectant, and other optional components are mixed together, the formulation is lyophilized. Many different freeze-dryers are available for this purpose such as Hull50™ (Hull, USA) or GT20™ (Leybold-Heraeus, Germany) freeze-dryers. In some embodiments, it may be desirable to lyophilize the conjugate formulation in the container in which reconstitution of the conjugate is to be carried out in order to avoid a transfer step. The container may be, e.g., a 3, 5, 10, 20, 50 or 100 cc vial.

At a desired later time, typically when it is time to administer the conjugate to a patient, the lyophilized formulation may be reconstituted with a diluent to provide a desired conjugate concentration in the reconstituted formulation, such as any of the conjugate concentrations provided herein. For example, the desired concentration may be 50-75 mg/mL, 75-100 mg/mL, 100-150 mg/mL, 1500-200 mg/mL, 200-250 mg/mL; 250-300 mg/mL; 300-350 mg/mL; 350-400 mg/mL; or 400-450 mg/mL. As noted above, such high conjugate concentrations in the reconstituted formulation are particularly useful where subcutaneous or ocular delivery of the reconstituted formulation is intended. However, for other routes of administration, such as intravenous administration, lower concentrations of the conjugate in the reconstituted formulation may be desired (e.g., 5-50 mg/mL, 10-40 mg/mL).

Reconstitution generally takes place at a temperature of 25° C., facilitating hydration, although other temperatures may be employed as desired. The time required for reconstitution will depend. e.g., on the type of diluent, amount of excipient(s) and conjugate components. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives include, but are not limited to, aromatic alcohols (such as benzyl or phenol alcohol). The amount of preservative employed can be determined by assessing different preservative concentrations for compatibility with the conjugate.

### V. Methods of Preparation

a. Methods of Preparing Nanolipoprotein Conjugates Comprising an Antigen-Binding Polypeptide

Another aspect of the present invention relates to methods of preparing conjugates comprising a polypeptide (e.g., an antigen-binding polypeptide). The methods generally involve a) providing a scaffold protein and one or more membrane-forming lipids under conditions allowing assembly (e.g., self-assembly) of a nanolipoprotein particle comprising a lipid bilayer of said membrane-forming lipids encircled by said scaffold protein, wherein one or more of the membrane-forming lipids presents a functionalized group on one or both surfaces of the particle; b) allowing the particle to contact an antigen-binding polypeptide having a C-terminally located complementary functional group that conjugates to said functionalized group at low pH; and c) optionally purifying the conjugate of said particle and said antigen-binding polypeptide.

In some embodiments, the membrane-forming lipids comprise a C₄₋₂₈ fatty-acyl (e.g., a C₁₆ fatty-acyl). In some embodiments, the C₄₋₂₈ fatty-acyl is covalently attached to a short peptide (e.g., a short peptide described herein). Such peptide-conjugated C₄₋₂₈ fatty-acyl can be produced as described in Example 7. Other methods of producing a peptide-conjugated C₄₋₂₈ fatty-acyl are known in the art.

In some embodiments, NLPs (including peptide-NLPs) are assembled by an in vitro translation method, where self-assembly of the NLPs is achieved while the scaffold protein is being translated from mRNA. In this process, expression system lysates are mixed with membrane-forming lipids and plasmid DNA encoding the scaffold protein. The reaction is allowed to proceed (e.g. for approximately 4-24 hours) until assembly occurs during expression of the scaffold protein.

In some embodiments, the method is performed at a predefined scaffold protein:lipid ratio, and/or conditions to increase the yield or to control the size and composition of the resulting NLP (or peptide-NLP). For example, scaffold protein:lipid ratios may be selected to provide NLPs (or peptide-NLPs) having ratios described above. In some embodiments, the method is performed at a predefined molar ratio of peptide conjugated C₄₋₂₈ fatty-acyl:other membrane forming lipids, e.g., a molar ratio of 10% (i.e., 10% peptide conjugated C₄₋₂₈ fatty-acyl and 90% other membrane forming lipids).

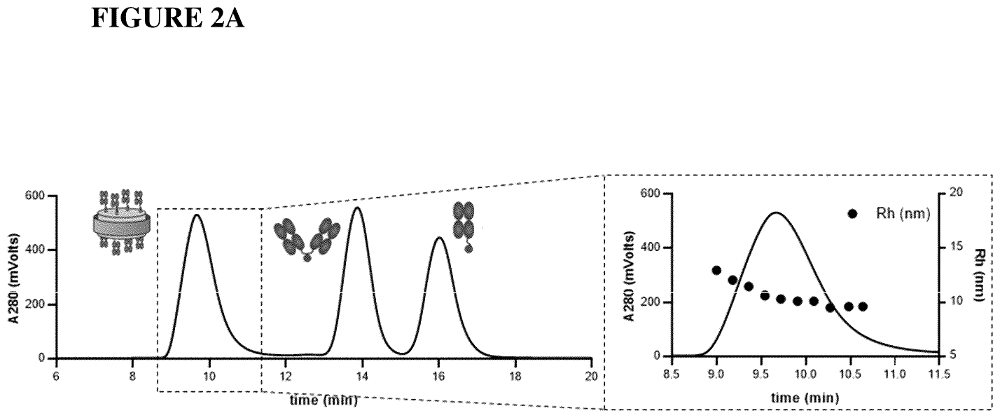

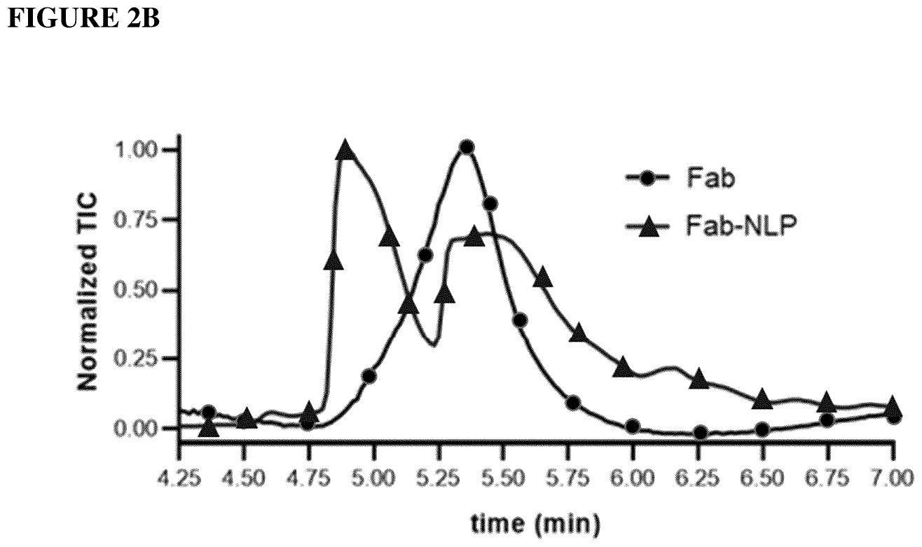

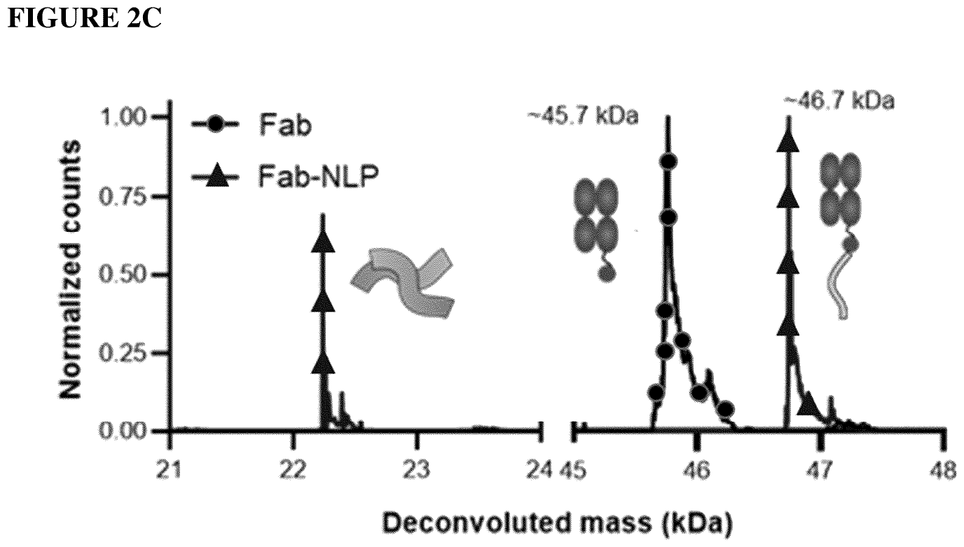

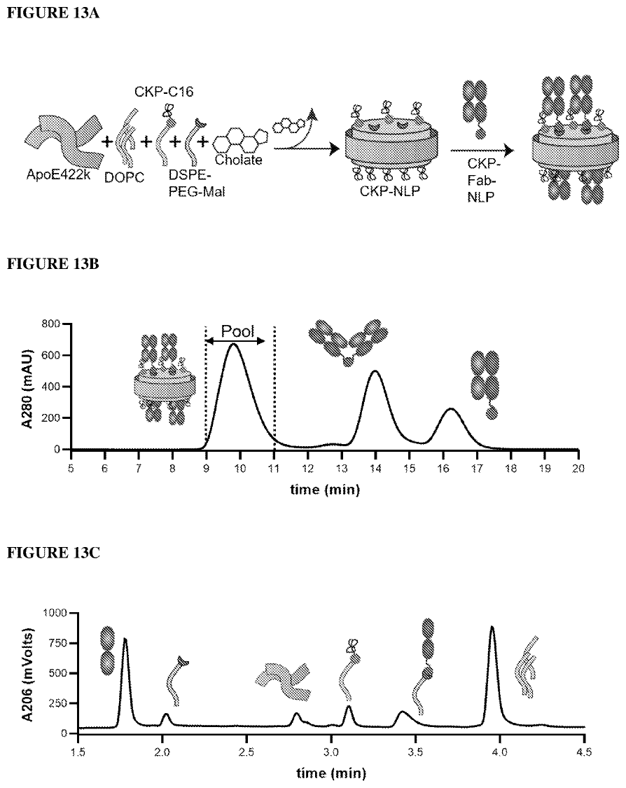

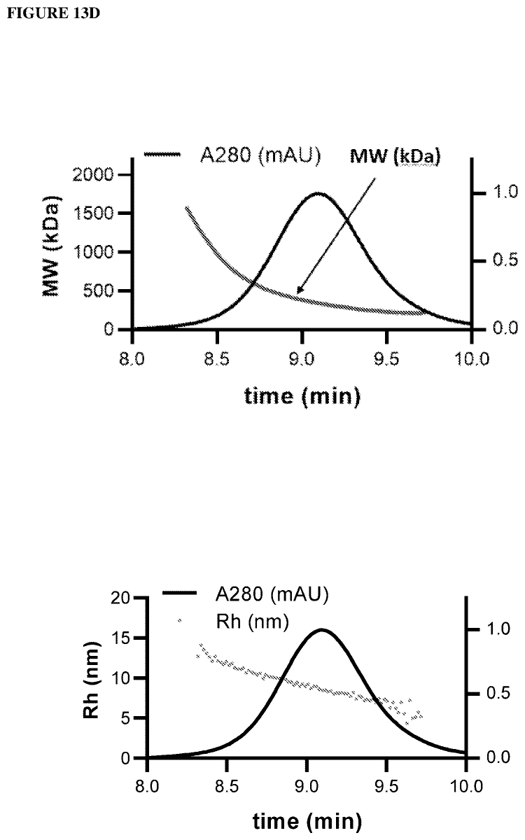

To form the conjugate of the present invention, the NLP (e.g., an NLP that is conjugated to a short peptide or an NLP that not conjugated to a short peptide) and antigen-binding polypeptide are contacted for a time and under conditions to allow conjugation between functionalized lipids and antigen-binding polypeptide functional groups. In a particular embodiment, NLPs assemble with a maleimide functionalized lipid (1,2-dioleoyl-sn-glycero-3-phospho-ethanolamine-N-[4-(p-maleimidomethyl)cyclohexane-carboxamide] (sodium salt)) (DOPE-MCC) were conjugated to Fabs containing a C-terminal cysteine (see, e.g., Examples 1-2, FIG. 2A-2C, and FIGS. 8A-8B). In another embodiment, NLPs comprising a peptide-conjugated C₄₋₂₈ fatty-acyl (e.g., a peptide conjugated C₁₆ fatty-acyl) assemble with a maleimide functionalized lipid (1,2-dioleoyl-sn-glycero-3-phospho-ethanolamine-N-[4-(p-maleimidomethyl)cyclohexane-carboxamide] (sodium salt)) (DOPE-MCC) were conjugated to Fabs containing a C-terminal cysteine. See Example 10 and FIG. 13A.

Typically, maleimide (or a maleimide derivative) reacts with a sulfhydryl in thiol-free buffered aqueous solutions, e.g., at a pH between 6.5 and 0.5 for 1-24 hours, to form a covalent thioether linkage. Maleimides are then quenched, e.g., at the completion of the reaction by the addition of free thiol. Surprisingly, in some embodiments, the present methods utilize a low pH for conjugating antigen-binding polypeptides comprising free thiols to maleimide functionalized lipids. The low pH may be 4.5-6.5, 5.5-6.5, 5-6, or 6. The low pH facilitates conjugation of the functionalized lipids to the antigen-binging polypeptides, rather than to the scaffold protein of the NLP (see, e.g., Example 2, FIGS. 1C-1D, triangles). Indeed, surprisingly maleimide reactive lipids conjugate to the apolipoprotein when NLPs are assembled at pH 7.4 (see, e.g., Example 2, FIG. 1B, FIGS. 1C-1D (circles) and FIG. 8A). However, this undesirable reaction was not observed when assembled at pH 6. These assembly conditions limit crosslinking between free lysines on scaffold proteins (e.g., apoE422k protein) and the maleimide functionalized lipids (e.g., DOPE-MCC).

Accordingly, in some embodiments as noted above, the functionalized group reacts with a complementary functional group on the polypeptide (e.g., antigen-binding polypeptide) and does not conjugate to other components of the NLP, such as not conjugating to the scaffold protein(s). For example, using low pH (e.g., pH 4.5-6.5, 5.5-6.5, 5-6, or 6) as described in the present methods, less than 25% or 20-25%, less than 20% or 10-20%, less than 15% or 10-15%, less than 10% or 5-10%, less than 5% or 3-5%, or 0% of the functionalized groups conjugate to scaffold protein in forming a conjugate of the invention. In some embodiments, 5%, 4%, 3%, 2%, 1%, or 0% of the functionalized groups conjugate to scaffold protein in forming a conjugate of the invention. In particular embodiments, the functionalized group is maleimide or a maleimide derivative and the functional group is free thiol group.

Accordingly, in some aspects, the invention provides a method of reducing lipid-scaffold protein conjugation when preparing a conjugate of a nanolipoprotein particle and an antigen-binding polypeptide.

In some embodiments, the methods do not require separation of the NLPs from unreacted components before conjugation to an antigen-binding polypeptide. That is, step b) (allowing the particle to contact an antigen-binding polypeptide having a C-terminally located complementary functional group that conjugates to said functionalized group at low pH) can follow step a) (providing a scaffold protein and a membrane-forming lipid under conditions allowing assembly of a nanolipoprotein particle comprising a lipid bilayer of said membrane-forming lipids encircled by said scaffold protein, wherein one or more of the membrane-forming lipids presents a functionalized group on one or both surfaces of the particle), without an intervening step to remove some or all unassembled membrane-forming lipids or some or all the unassembled scaffold protein. Surprisingly as described herein when particular ratios of functionalized membrane-forming lipids to scaffold protein are used, few functionalized membrane-forming lipids remain unassembled into NLPs, and those few that remain unassembled become stuck to reaction vessel walls, leaving none, few, or very few available to react with functional groups of the antigen-binding polypeptide. Accordingly, in some embodiments as noted above, the scaffold protein and membrane-forming lipids are combined in a molar ratio of from 1:50 to 1:100; from 1:60 to 1:90; or from 1:70 to 1:80, and no step of removing unassembled lipid is needed or performed before conjugation to an antigen-biding polypeptide (e.g., a Fab). In some embodiments, the scaffold protein and membrane-forming lipids are in a molar ratio of 1:60, 1:70, 1:75, 1:80, 1:85, 1:90, or 1:100, and no step of removing unassembled lipid is needed or performed before conjugation to an antigen-biding polypeptide (e.g., a Fab). In a specific embodiment, the scaffold protein and membrane-forming lipids are in a molar ratio of 1:80, and no step of removing unassembled lipid is needed or performed before conjugation to an antigen-biding polypeptide (e.g., a Fab).

In some embodiments, wherein the NLP comprises a peptide-conjugated C₄₋₂₈ fatty-acyl, the NLP-peptide conjugate is purified, e.g., via size-exclusion chromatography, following self-assembly and prior to conjugation to an antigen-biding polypeptide (e.g., a Fab), as described above.

b. Methods of Preparing Nanolipoprotein Conjugates Comprising a Short Peptide

In a related aspect, provided are methods of preparing conjugates comprising a short peptide (i.e., peptide-NLP conjugates). Also provided are methods of increasing avidity, activity, and/or potency of a short peptide of 20-60 amino acids. In some embodiments, the method comprises providing a scaffold protein and a membrane-forming lipid under conditions allowing assembly (e.g., self-assembly) of a nanolipoprotein particle comprising a lipid bilayer of said membrane-forming lipids encircled by said scaffold protein; wherein one or more of said membrane-forming lipids comprises a C₄₋₂₈ fatty-acyl (e.g., a C₁₆ fatty-acyl) conjugated to said short peptide (e.g., a peptide between 20 and 60 amino acids in length). In some embodiments, the method further comprises purifying the peptide-NLP conjugate (e.g., via size exclusion chromatography). In some embodiments, the short peptide is a cystine-knot peptide (CKP), e.g., a CKP described elsewhere herein. In some embodiments, short peptide is CKP variant. In some embodiments, the CKP variant comprises one or more amino acid insertions, deletions, and/or substitutions in one or more loop sequences relative to the corresponding one or more loop sequences of a wild type CKP. Additionally or alternatively, in some embodiments, the CKP variant one or more amino acid insertions, deletions, and/or substitutions at the N terminus relative to a wild type CKP. Additionally or alternatively, in some embodiments, the CKP variant one or more amino acid insertions, deletions, and/or substitutions at the C-terminus relative to a wild type CKP. Additionally or alternatively, in some embodiments, the CKP variant comprises a chemical modification at the N-terminus relative to a wild type CKP. Additionally or alternatively, in some embodiments, the CKP variant comprises a chemical modification at the C-terminus relative to a wild type CKP. In some embodiments, the membrane-forming lipids comprise a peptide-C₄₋₂₈ fatty-acyl conjugate and at least one more of DMPC, DOPC, DOPS, DOPE, DPPC (e.g., a peptide-C₁₆ fatty-acyl conjugate and DOPC) in the molar ratio of 1:3 to 1:15, or 1:6 to 1:12, or 1:9. In some embodiments, said conjugate comprises 1-100, 10-90, 20-80, 30-70, 40-60, or 60 molecules of said short peptide. In some embodiments, the conjugate comprises at least two different peptides (e.g., as described in further detail elsewhere herein). FIG. 10A depicts a schematic of the assembly of an exemplary NLP comprising a short peptide.

In some embodiments, one or more of said membrane-forming lipids presents a functionalized group on one or both surfaces of said peptide conjugate, and the method further comprises the step of allowing said peptide-NLP conjugate to contact peptide (e.g., an antigen-binding polypeptide, such as a Fab or Fab-like molecule) having a C-terminally located complementary functional group that conjugates to said functionalized group at low pH in the range of 4.5 to 6.5 pH. In some embodiments, the conjugation of the polypeptide (e.g., antigen-binding polypeptide) to the peptide-NLP conjugate follows assembly (e.g., self-assembly) of the peptide-NLP conjugate without an intervening step to remove some or all unassembled membrane-forming lipids. In some embodiments the conjugation of the antigen-binding polypeptide to the peptide conjugate follows assembly (e.g., self-assembly) of the peptide conjugate without an intervening step to enrich the peptide conjugate. In some embodiments, the functionalized group is a maleimide derivative and wherein said functional group is a cysteine thiol group, optionally wherein said cysteine amino acid residue forms a hinge disulfide bond in the antibody from which the antigen-binding polypeptide is derived (such as Cys-226 or Cys-227). In some embodiments, the functionalized group does not conjugate to said scaffold protein at said low pH. In some embodiments, said low pH is a pH of 5.5 to 6.5, a pH of 5-6, or a pH of 6. In some embodiments, a spacer connects said functionalized group to said membrane-forming lipid and/or a spacer connects said complementary functional group to said antigen-binding polypeptide. In some embodiments, the spacer is a PEG spacer that connects said functionalized group to said membrane-forming lipid. In some embodiments, the PEG spacer has a MW of 1000-3000, 1500-2500, 1900-2200, or 2000.

The present methods herein find industrial applicability in the production of the conjugates, as described herein (e.g., peptide-NLP conjugates, peptide-NLP-polypeptide conjugates, and NLP-polypeptide conjugates). Such conjugates find use in, for example, in vitro, ex vivo, and in vivo therapeutic methods. Provided herein are various methods based on using one or more of the conjugates of the invention.

### VI. Treatment Methods and Uses

Another aspect of the invention relates to use of the conjugates described herein (e.g., peptide-NLP conjugates, peptide-NLP-polypeptide conjugates, and NLP-polypeptide conjugates) for treatment. The disease, disorder, pathological condition to be treated will inform the selection of antigen-binding polypeptide(s) and/or short peptide for use in the conjugate. More particularly, in certain diseases, disorders, or conditions, it is necessary or desirable to use multivalent and/or multi-specific antigen-binding constructs conjugate (e.g., a NLP-Fab conjugate, a NLP-Fab-like molecule conjugate, a peptide-NLP-Fab conjugate, or a peptide-NLP-Fab-like molecule conjugate). The conjugates of the present invention (e.g., peptide-NLP conjugates, peptide-NLP-polypeptide conjugates, and NLP-polypeptide conjugates) provide a versatile platform for multivalent and/or multi-specific antigen-binding polypeptides and/or short peptide, as described above, which can be used for a variety of purposes, e.g., as therapeutics, prophylactics, delivery, and diagnostics.

As used herein, “treatment” (and grammatical variations thereof such as “treat” or “treating”) refers to clinical intervention in an attempt to alter the natural course of a disease in a subject being treated to bring about a beneficial effect, and can be performed either prophylactically or during the course of a disease. Beneficial effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, remission, improved prognosis, and the like. In some embodiments, an NLP conjugate comprising an antigen-binding polypeptide (including, e.g., a Fab or Fab-like molecule) and/or a short peptide (e.g., a CKP or a CKP variant) is used to delay development of a disease or to slow the progression of a disease, or to reduce the likelihood, frequency, and/or severity of relapse of a disease.

Generally, the conjugate (e.g., peptide-NLP conjugate, peptide-NLP-polypeptide conjugate, and/or NLP-polypeptide conjugate) is provided in a therapeutically effective amount to the subject requiring benefit therefrom. A “therapeutically effective amount” of an agent e.g., in a pharmaceutical composition, refers to an amount that, upon administration to a subject, provides an effective amount of the agent (e.g., an antigen-binding polypeptide (e.g., a Fab or Fab-like molecule), and/or a short peptide (e.g., CKP or CKP variant), and/or other therapeutic or diagnostic agent) to a target cell or tissue in order to result in a physiological change in the cell or tissue, thereby producing a beneficial effect to the subject receiving the pharmaceutical composition. A pharmaceutical composition generally will be administered at dosages and for periods of time to achieve a desired therapeutic or prophylactic result.

a. Treatment of Malignant Disease

In some embodiments, the antigen-binding polypeptide (e.g., a Fab or Fab-like molecule) binds a target that plays a role in angiogenesis and/or tumor growth; and the conjugate (e.g., an NLP-polypeptide conjugate) finds use in inhibiting angiogenesis and/or tumor growth to treat a malignant disease. In some embodiments, a conjugate that finds use in inhibiting angiogenesis and/or tumor growth further comprises a short peptide that binds a target that plays a role in angiogenesis and/or tumor growth (i.e., a peptide-NLP-polypeptide conjugate). In some embodiments, the short peptides binds the same target as the antigen-binding polypeptide. In some embodiments, the short peptide binds a different target than the antigen-binding polypeptide. In some embodiments, the short peptide exhibits an activity (e.g., a therapeutic activity) that is complementary to or synergistic with the activity (e.g., therapeutic activity) of the antigen-binding polypeptide. In particular embodiments, the malignant disease is selected from cancer of epithelial, endothelial or mesothelial origin and cancer of the blood. Conjugates of the invention can be used for the treatment of tumors, including pre-cancerous, non-metastatic, metastatic, and cancerous tumors (e.g., early stage cancer), or for the treatment of a subject at risk for developing cancer. In further aspects, the antigen-binding polypeptide of the conjugate (e.g., a Fab) plays a targeting or stabilizing role. e.g., targeting a stabilized NLP-Fab conjugate or a stabilized peptide-NLP-Fab conjugate to antigens associated with a disease, discussed in more detail below.

In particular embodiments, the cancer or carcinoma is selected from the group consisting of gastrointestinal cancer, pancreatic cancer, cholangiocellular cancer, lung cancer, breast cancer, ovarian cancer, skin cancer, oral cancer, gastric cancer, cervical cancer, B and T cell lymphoma, myeloid leukemia, ovarian cancer, leukemia, lymphatic leukemia, nasopharyngeal carcinoma, colon cancer, prostate cancer, renal cell cancer, head and neck cancer, skin cancer (melanoma), cancers of the genitourinary tract, e.g., testis cancer, ovarian cancer, endothelial cancer, cervix cancer and kidney cancer, cancer of the bile duct, esophagus cancer, cancer of the salivary glands and cancer of the thyroid gland or other tumorous diseases like haematological tumors, gliomas, sarcomas or osteosarcomas.

In the case of tumor (e.g., a cancerous tumor), the therapeutically effective amount of an agent may reduce the number of cancer cells; reduce the primary tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the disorder. For cancer therapy, efficacy in vivo can, for example, be measured by assessing the duration of survival, time to disease progression (TTP), the response rates (RR), duration of response, and/or quality of life.

By “reduce or inhibit” is meant the ability to cause an overall decrease, e.g., of 20%, 50%, 75%, 85%, 90%, or 95%. Reduce or inhibit can refer to the effect on one or more symptoms of the disorder being treated, the presence or size of metastases, the size of the primary tumor, or the size or number of the blood vessels in angiogenic disorders.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia), or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further separated into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors.

In some embodiments, the NLP conjugate comprises at least one molecule of at least two antigen-binding polypeptides (including e.g., one or more Fabs or Fab-like molecules) that bind a pair of clinically relevant targets, as described above. For example, the clinically relevant pair may comprise at least one of a T cell marker, a co-stimulatory receptor, or NK cell marker; and a tumor antigen, such as any one or more of the tumor antigens disclosed herein and/or known in the art. In some embodiments, the NLP conjugate comprises (such as further comprises) a short peptide (e.g., a CKP or a CKP variant). In some embodiments the short peptide exhibits an activity (e.g., a binding activity and/or a therapeutic activity) that is the same as that of an antigen-binding polypeptide in the conjugate. In some embodiments the short peptide exhibits an activity (e.g., a binding activity and/or a therapeutic activity) that is the different from (e.g., complementary to or synergistic with) that of an antigen-binding polypeptide in the conjugate. In some embodiments, the conjugate comprises at least one molecule each of at least two different short peptides.

In a specific example, the NLP conjugate comprises a first antigen-binding polypeptide (e.g., a first Fab) that binds CD3; and at a second antigen-binding polypeptide (e.g., a second Fab) that binds targeting CD19 (a tumor antigen), for use in treatment of acute lymphoblastic leukemia. In another specific example, the NLP conjugate comprises a first antigen-binding polypeptide (e.g., a first Fab) that binds CD3; and a second antigen-binding polypeptide (e.g., a second Fab) that binds EpCAM (a tumor antigen), for use in the treatment of carcinomas of various origins including colon, rectum, ovarian, gastric, esophagus, lung, pancreas, breast and head and neck.

Particular embodiments provide an NLP conjugate of the invention comprising a first antigen-binding polypeptide (e.g., a Fab) that binds HER1, preferably human HER1, and optionally a second antigen-binding polypeptide (e.g., a second Fab) that binds at least one of a NK cell marker, a T-cell marker, or a co-stimulatory receptor, for use in the treatment of gastrointestinal cancer, pancreatic cancer, cholangiocellular cancer, lung cancer, breast cancer, ovarian cancer, skin cancer and/or oral cancer.

Particular embodiments provide an NLP conjugate of the invention comprising a first antigen-binding polypeptide (e.g., a Fab) that binds HER2, preferably human HER2, and optionally a second antigen-binding polypeptide (e.g., a second Fab) that binds at least one of a NK cell marker, a T-cell marker, or a co-stimulatory receptor, for use in the treatment of gastric cancer, breast cancer and/or cervical cancer.

Particular embodiments provide an NLP conjugate of the invention comprising a first antigen-binding polypeptide (e.g., a Fab) that binds HER3, preferably human HER3, and optionally a second antigen-binding polypeptide (e.g., a second Fab) that binds at least one of a NK cell marker, a T-cell marker, or a co-stimulatory receptor, for use in the treatment of gastric cancer and/or lung cancer.

Particular embodiments provide an NLP conjugate of the invention comprising a first antigen-binding polypeptide (e.g., a Fab) that binds CEA, preferably human CEA, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds p95, preferably human p95, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds BCMA, preferably human BCMA, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds MSLN, preferably human MSLN, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds MCSP, preferably human MCSP, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds CD19, preferably human CD19, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds CD20, preferably human CD20, and optionally a second antigen-binding polypeptide (e.g., a second Fab) that binds at least one of a NK cell marker, a T-cell marker, or a co-stimulatory receptor, for use in the treatment of B-cell lymphoma and/or T cell lymphoma.

Particular embodiments provide an NLP conjugate of the invention comprising a first antigen-binding polypeptide (e.g., a Fab) that binds CD22, preferably human CD22, and optionally a second antigen-binding polypeptide (e.g., a second Fab) that binds at least one of a NK cell marker, a T-cell marker, or a co-stimulatory receptor, for use in the treatment of B-cell lymphoma and/or T cell lymphoma.

Particular embodiments provide an NLP conjugate of the invention comprising a first antigen-binding polypeptide (e.g., a Fab) that binds CD38, preferably human CD38, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds CD52Flt3, preferably human CD52Flt3, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds FolR1, preferably human FolR1, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds Trop-2, preferably human Trop-2, and optionally a second antigen-binding polypeptide (e.g., a second Fab) that binds at least one of a NK cell marker, a T-cell marker, or a co-stimulatory receptor, for use in the treatment of gastrointestinal cancer, pancreatic cancer, cholangiocellular cancer, lung cancer, breast cancer, ovarian cancer, skin cancer, glioblastoma and/or oral cancer.

Particular embodiments provide an NLP conjugate of the invention comprising a first antigen-binding polypeptide (e.g., a Fab) that binds CA-12-5, preferably human CA-12-5, and optionally a second antigen-binding polypeptide (e.g., a second Fab) that binds at least one of a NK cell marker, a T-cell marker, or a co-stimulatory receptor, for use in the treatment of ovarian cancer, lung cancer, breast cancer and/or gastrointestinal cancer.

Particular embodiments provide an NLP conjugate of the invention comprising a first antigen-binding polypeptide (e.g., a Fab) that binds HLA-DR, preferably human HLA-DR, and optionally a second antigen-binding polypeptide (e.g., a second Fab) that binds at least one of a NK cell marker, a T-cell marker, or a co-stimulatory receptor, for use in the treatment of gastrointestinal cancer, leukemia and/or nasopharyngeal carcinoma.

Particular embodiments provide an NLP conjugate of the invention comprising a first antigen-binding polypeptide (e.g., a Fab) that binds MUC-1, preferably human MUC-1, and optionally a second antigen-binding polypeptide (e.g., a second Fab) that binds at least one of a NK cell marker, a T-cell marker, or a co-stimulatory receptor, for use in the treatment cancer of colon cancer, breast cancer, ovarian cancer, lung cancer and/or pancreatic cancer.

Particular embodiments provide an NLP conjugate of the invention comprising a first antigen-binding polypeptide (e.g., a Fab) that binds A33, preferably human A33, and optionally a second antigen-binding polypeptide (e.g., a second Fab) that binds at least one of a NK cell marker, a T-cell marker, or a co-stimulatory receptor, for use in the treatment of colon cancer.

Particular embodiments provide an NLP conjugate of the invention comprising a first antigen-binding polypeptide (e.g., a Fab) that binds PSMA, preferably human PSMA, and optionally a second antigen-binding polypeptide (e.g., a second Fab) that binds at least one of a NK cell marker, a T-cell marker, or a co-stimulatory receptor, for use in the treatment of prostate cancer.

Particular embodiments provide an NLP conjugate of the invention comprising a first antigen-binding polypeptide (e.g., a Fab) that binds PSCA, preferably human PSCA, and optionally a second antigen-binding polypeptide (e.g., a second Fab) that binds at least one of a NK cell marker, a T-cell marker, or a co-stimulatory receptor, for use in the treatment cancer of epithelial, endothelial or mesothelial origin and cancer of the blood.

Particular embodiments provide an NLP conjugate of the invention comprising a first antigen-binding polypeptide (e.g., a Fab) that binds transferring-receptor, preferably human transferring-receptor, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds tenascin, preferably human tenascin, and optionally a second antigen-binding polypeptide (e.g., a second Fab) that binds at least one of a NK cell marker, a T-cell marker, or a co-stimulatory receptor, for use in the treatment of cancer of epithelial, endothelial or mesothelial origin and cancer of the blood.

Particular embodiments provide an NLP conjugate of the invention comprising a first antigen-binding polypeptide (e.g., a Fab) that binds CA-IX, preferably human CA-IX, and optionally a second antigen-binding polypeptide (e.g., a second Fab) that binds at least one of a NK cell marker, a T-cell marker, or a co-stimulatory receptor, for use in the treatment of renal cancer.

Conjugates (e.g., peptide-NLP conjugates, peptide-NLP-polypeptide conjugates, and/or NLP-polypeptide conjugates), pharmaceutical compositions, or formulations of the present invention may be administered alone or in combination with another therapeutic agent or detectable agent/label. The term “in combination” does not restrict the order in which the components of the treatment regimen are administered to the subject. For example, the pharmaceutical composition or medicament may be administered before, at the same time, or after administration of the other therapy. “In combination” also does not restrict the timing between the administrations. Thus, when the two components are not administered simultaneously or concurrently, the administrations may be separated by 1 minute, 5 minutes, 15 minutes, 30 minutes, 45 minutes, 1 hour, 2 hours, 4 hours, 6 hours, 12 hours, 24 hours, 48 hours or 72 hours or by any suitable time differential readily determined by one of skill in art and/or described herein.

The additional therapy may include one or more treatment protocols suitable for the treatment or prevention of a disease, or a symptom thereof, as described herein or known in the art. Examples of such treatment protocols include but are not limited to, administration of pain medications, administration of chemotherapeutics, surgical handling of the disease or a symptom thereof. Examples of additional therapeutics for administration in combination with a conjugate of the invention, for example in the context of treating a malignant disease, include drugs acting on the gastro-intestinal system, drugs acting as cytostatics, drugs preventing hyperuricemia, drugs inhibiting immune reactions (e.g. corticosteroids), drugs acting on the circulatory system and/or agents such as T cell co-stimulatory molecules or cytokines known in the art.

The invention further envisages the co-administration protocols with molecules capable of providing an activation signal for immune effector cells, for cell proliferation, or for cell stimulation. Said molecule may be, e.g., a primary activation signal for T cells (e.g. a costimulatory molecule: molecules of B7 family, Ox40L, 4.1 BBL, CD40L, anti-CTLA-4, anti-PD-1), or a cytokine interleukin (e.g., IL-2).

b. Treatment of Ocular Diseases

In certain embodiments, the conjugates (e.g., peptide-NLP conjugates, peptide-NLP-polypeptide conjugates, and/or NLP-polypeptide conjugates) of the present invention find use in treating an ocular disease or disorder in a subject. In certain embodiments, the subject is suspected of having or at risk for having an ocular disease or disorder characterized by abnormal angiogenesis and/or abnormal vascular permeability. In certain embodiments, the subject has been diagnosed with an ocular disease or disorder characterized by abnormal angiogenesis and/or abnormal vascular permeability.

In some embodiments, the ocular disease or disorder is an ocular vascular proliferative disease, such as an ocular vascular proliferative disease selected from the group consisting of diabetic blindness, retinopathies, primarily diabetic retinopathy, age-related macular degeneration (AMD), proliferative diabetic retinopathy (PDR), retinopathy of prematurity (ROP), choroidal neovascularization (CNV), diabetic macular edema, pathological myopia, von Rippel-Lindau disease, histoplasmosis of the eye, retinal vein occlusion (both branched retinal vein occlusion (BRVO) and central retinal vein occlusion (CRVO), corneal neovascularization, retinal neovascularization, and rubeosis. In certain embodiments, the corneal neovascularization results in infection of the eye, inflammation in the eye, trauma to the eye (including chemical burns), or loss of the limbal stern cell barrier. In specific embodiments, the corneal neovascularization results from herpetic keratitis, trachoma, or onchocerciasis.

In particular embodiments, conjugates for treatment of an ocular disease comprise an antigen-binding polypeptide (e.g., a Fab) that binds at least one of VEGF (preferably human VEGF), Factor D (preferably human Factor D), Tie2 (preferably human Tie2), and DR4 (preferably human DR4). In more particular embodiments, conjugates for treatment of an ocular disease comprise an antigen-binding polypeptide (e.g., a Fab) that binds VEGF, e.g., VEGF-A, preferably human VEGF-A. In even more particular embodiments, conjugates for treatment of an ocular disease comprise a first antigen-binding polypeptide (e.g., a first Fab) that binds VEGF (e.g., VEGF-A, preferably human VEGF-A); and a second antigen-binding polypeptide (e.g., a second Fab) that binds one or more of Factor D (preferably human Factor D), Tie2 (preferably human Tie2), and DR4 (preferably human DR4). In a specific example, the conjugate comprises a first antigen-binding polypeptide (e.g., a first Fab) that binds VEGF-A; and a second antigen-binding polypeptide (e.g., a second Fab) that binds Ang-2, e.g., for treatment of diabetic macular edema. In some embodiments, such conjugate comprises (e.g., further comprises) at least one short peptide (e.g., a CKP or CKP variant) that binds a target listed above. In some embodiments, the Fab and the short peptide bind to the same target. In some embodiments, the Fab and the short peptide bind to different targets. In some embodiments, the Fab and the short peptide exhibit complementary or synergistic therapeutic activities.

In certain embodiments, conjugates of the present invention for treating an ocular disease disorder are administered in combination with a second therapeutic agent. For patients where the ocular disease or disorder is triggered by an inflammatory response, combination therapy with an antiinflammatory agent can be considered. Patients who suffer from an ocular disease or disorder secondary to bacterial, viral, fungal or acanthamoebal infection, combination therapy may comprise administration of an antimicrobial agent and optionally an antiinflammatory agent.

In certain embodiments, conjugates of the present invention for treating an ocular disease disorder are administered in combination with a second therapy, such as laser photocoagulation therapy (LPT) and/or photodynamic therapy (PDT), where a light-activated molecule (e.g., verteporfin) can be made to locally damage neovascular endothelium by light activation (see, e.g., WO 2014/033184), In certain embodiments, the second therapy is diathermy and cautery, wherein vessels are occluded either by applying a coagulating current through a unipolar diathermy unit or by thermal cautery using an electrolysis needle.

c. Treatment of Other Diseases

Conjugates of the present invention may also find use in treatment of other diseases, e.g., where therapeutic benefit requires or is enhanced using multimeric and/or multi-specific formats of antigen-binding polypeptides (and, in some embodiments short peptides), particularly stable, high avidity formats, and/or where high concentrations of antigen-binding polypeptides (and, in some embodiments short peptides) are to be administered in small volumes, as described herein.

For example, conjugates of the invention can be used for the treatment of allergic or inflammatory disorders, or for the treatment of autoimmune disease, or for the treatment of a subject at risk for developing an allergic or inflammatory disorder, or an autoimmune disease.

Other subjects that are candidates for receiving compositions provided herein have, or are at risk for developing, abnormal proliferation of fibrovascular tissue, acne rosacea, acquired immune deficiency syndrome, artery occlusion, atopic keratitis, bacterial ulcers, Bechets disease, blood borne tumors, carotid obstructive disease, choroidal neovascularization, chronic inflammation, chronic retinal detachment, chronic uveitis, chronic vitritis, contact lens overwear, corneal graft rejection, corneal neovascularization, corneal graft neovascularization, Crohn's disease, Eales disease, epidemic keratoconjunctivitis, fungal ulcers, Herpes simplex infections, Herpes zoster infections, hyperviscosity syndromes, Kaposi's sarcoma, leukemia, lipid degeneration, Lyme's disease, marginal keratolysis, Mooren ulcer, Mycobacteria infections other than leprosy, myopia, ocular neovascular disease, optic pits, Osler-Weber syndrome (Osler-Weber-Rendu), osteoarthritis, Paget's disease, pars planitis, pemphigoid, phylectenulosis, polyarteritis, post-laser complications, protozoan infections, pseudoxanthoma elasticum, pterygium keratitis sicca, radial keratotomy, retinal neovascularization, retinopathy of prematurity, retrolental fibroplasias, sarcoid, scleritis, sickle cell anemia, Sogren's syndrome, solid tumors, Stargart's disease, Steven's Johnson disease, superior limbic keratitis, syphilis, systemic lupus, Terrien's marginal degeneration, toxoplasmosis, tumors of Ewing sarcoma, tumors of neuroblastoma, tumors of osteosarcoma, tumors of retinoblastoma, tumors of rhabdomyosarcoma, ulcerative colitis, vein occlusion, Vitamin A deficiency, Wegener's sarcoidosis, undesired angiogenesis associated with diabetes, parasitic diseases, abnormal wound healing, hypertrophy following surgery, injury or trauma (e.g., acute lung injury/ARDS), inhibition of hair growth, inhibition of ovulation and corpus luteum formation, inhibition of implantation, and inhibition of embryo development in the uterus.

Particular embodiments provide a conjugate of the invention comprising a first antigen-binding polypeptide (e.g., a Fab) that binds factor IXa, preferably human factor IXa, and a second antigen-binding polypeptide (e.g., a second Fab) that binds Factor X, for use for use in treating hemophilia, e.g., hemophilia A. Hemophilia A is a severe inherited bleeding disorder, where the patient suffers extensive bleeding due to a deficiency or dysfunction of the procoagulant cofactor protein, factor VIII. In some embodiments, binding of a bispecific conjugate of the invention to both factor IXa and factor X mimics some of the function of activated factor VIII, and can find use in treatment of the disease. See. e.g., Nogami et al., New therapies using nonfactor products for patients with hemophilia and inhibitors; Blood; 2019; 133(5):399-406. In some embodiments, such conjugate comprises (e.g., further comprises) a short peptide (e.g., a CKP or CKP variant) that binds a target listed above. In some embodiments, the Fab and the short peptide bind to the same target. In some embodiments, the Fab and the short peptide bind to different targets. In some embodiments, the Fab and the short peptide exhibit complementary or synergistic therapeutic activities.

### VII. Delivery Methods and Uses

Another aspect of the invention provides conjugates (e.g., peptide-NLP conjugates, peptide-NLP-polypeptide conjugates, and/or NLP-polypeptide conjugates) for use in the delivery of one or more additional therapeutic or diagnostic agents. The conjugate may be delivered in a composition, e.g., as a pharmaceutical composition as described herein. In some embodiments, the conjugate is provided in a low viscosity formulation, providing a high concentration of the NLP-polypeptide conjugate or the peptide-NLP-polypeptide conjugate e.g., as described above. In some embodiments, the antigen-binding polypeptide serves as a stabilizing agent, stabilizing the conjugate comprising, e.g., a short peptide, and/or one or more other therapeutic agents or diagnostic agents, e.g., to increase shelf life and/or serum half-life, in particular, e.g., facilitating lyophilization of the conjugate (without substantial loss of activity); and/or facilitating manufacture of low viscosity formulations comprising high conjugate concentrations, again, e.g., as discussed above. In a specific embodiment, a conjugate of the invention allows specific or targeted delivery across the blood-brain barrier, allowing its cargo (e.g. one or more short peptides and/or one or more additional therapeutic or diagnostic agents) to access targets in the brain or central nervous system, again, e.g., as discussed above. Use of conjugates of the invention, in certain embodiments, to deliver specific therapeutic or diagnostic agents, as well to effect translocation of blood-brain barrier, is described in more detail below

a. Delivery of Therapeutic Agents

In some embodiments, the antigen-binding polypeptide comprises the therapeutic agent, as described above. In some embodiments, the antigen-binding polypeptide serves more as a targeting moiety, directing the conjugate to certain cells or tissues based on affinity to specific antigens, and the conjugate further comprises one or more therapeutic agents or diagnostic agents, e.g., a biologically active agent or a detectable agent, respectively, in addition to the antigen-binding polypeptide. While NLPs have been explored for select applications and drug cargo, including solubilizing membrane proteins (21-26), generation of protein pore complexes (27), hydrophobic drugs (14, 28, 29), proteins (14, 20, 30, 31), cancer neoantigens (32) and immune modulation drugs (17), the present disclosure provides conjugates with antigen-binding polypeptides (in particular, Fabs) with surprising characteristics including, as discussed herein, stability under physiological conditions, good manufacturability, ability to be lyophilized and reconstituted without loss (or without significant loss) of activity, and ability to be concentrated in low viscosity formulations. In some embodiments, the conjugate comprises (such as further comprises) a short peptide (e.g., a CKP or a CKP variant). In some embodiments the short peptide exhibits an activity (e.g., a binding activity and/or a therapeutic activity) that is the same as that of an antigen-binding polypeptide in the conjugate. In some embodiments the short peptide exhibits an activity (e.g., a binding activity and/or a therapeutic activity) that is the different from (e.g., complementary to or synergistic with) that of an antigen-binding polypeptide in the conjugate. In some embodiments, the conjugate comprises at least one molecule each of at least two different short peptides. All of the references cited in this paragraph can be found below Example 5.

Accordingly, in some aspects the invention provides a method of delivering a biologically active agent or a detectable agent to an individual in need thereof, in a stable and/or low viscosity liquid formulation, comprising administering to an individual a liquid formulation comprising a conjugate of the invention that further comprises biologically active agent and/or a detectable agent. In some embodiments, the antigen-binding polypeptide (e.g., a Fab) serves one or more of the functions recited herein, e.g., functioning as both a targeting agent and a stabilizing agent to a conjugate comprising the antigen-binding polypeptide along with another therapeutic or diagnostic agent. In some embodiments, the antigen-binding polypeptide is a Fab. In some embodiments, the antigen-binding polypeptide is a Fab-like molecule, that is, a polypeptide having a similar size and/or conformational shape as a Fab though not necessarily capable of binding to an antigen. In some embodiments, the conjugate comprises (such as further comprises) a short peptide (e.g., a CKP or a CKP variant). In some embodiments the short peptide exhibits an activity (e.g., a binding activity and/or a therapeutic activity) that is the same as that of an antigen-binding polypeptide in the conjugate. In some embodiments the short peptide exhibits an activity (e.g., a binding activity and/or a therapeutic activity) that is the different from (e.g., complementary to or synergistic with) that of an antigen-binding polypeptide in the conjugate. In some embodiments, the conjugate comprises at least one molecule each of at least two different short peptides.

The additional therapeutic or diagnostic agent will associate with the conjugates described herein by covalent or noncovalent association. For example, a hydrophobic therapeutic or diagnostic agent may associate with the nonpolar region of the lipid bilayer, while any hydrophilic portions extend to either or both the interior and exterior regions. In some embodiments, the therapeutic or diagnostic agent is functionalized to interact with the same or different functionalized groups on the membrane-forming lipids of the NLPs, either directly and/or via a spacer on the agent and/or the lipid, e.g., as described herein (see also, e.g., US 2009/0311276A1, US 2019/0142752A1, US 2018/0318218A1, US 2019/0307692A1, US 2010/0092567A1, and US 2011/0059549A1). In some embodiments, the therapeutic or diagnostic agent may associate with the antigen-binding polypeptide (e.g., Fab) of the conjugate, either directly and/or via a spacer, e.g., as described herein. Alternatively, or in addition, the conjugate may further comprise one or more antigen-binding polypeptides (e.g., Fabs) that bind the therapeutic or diagnostic agent (or bind a hapten of the therapeutic or diagnostic agent), allowing for pre-targeted delivery. In pre-targeted delivery, a conjugate of the invention is administered to a subject, where the conjugate comprises an antigen-binding polypeptide that binds a tumor marker (e.g., CEA, CD38), followed by the therapeutic or diagnostic agent. In some embodiments, wherein the conjugate comprises a short peptide, the therapeutic or diagnostic agent may associate with the short peptide of the conjugate.

In particular embodiments, a conjugate of the invention (e.g., a peptide-NLP conjugate, peptide-NLP-polypeptide conjugate, and/or NLP-polypeptide conjugate) targets a specific type of cancer. For example, gastrointestinal cancer, pancreatic cancer, cholangiocellular cancer, lung cancer, breast cancer, ovarian cancer, skin cancer and/or oral cancer may be targeted with a conjugate comprising one or more antigen-binding polypeptides (e.g., a Fabs) and/or one or more short peptides that bind (human) EpCAM (as the tumor-specific antigen naturally occurring on the surface of a tumor cell). Gastrointestinal cancer, pancreatic cancer, cholangiocellular cancer, lung cancer, breast cancer, ovarian cancer, skin cancer and/or oral cancer may be targeted with a conjugate comprising one or more antigen-binding polypeptides (e.g., Fabs) and/or one or more short peptides that bind HER1, preferably human HER1 or Trop-2, preferably human Trop-2. Furthermore, gastrointestinal cancer, pancreatic cancer, cholangiocellular cancer, lung cancer, breast cancer, ovarian cancer, skin cancer, glioblastoma and/or oral cancer may be targeted with a conjugate comprising one or more antigen-binding polypeptides (e.g., Fabs) that bind MCSP, preferably human MCSP; FOLR1, preferably human FOLR1; PSCA, preferably human PSCA; EGFRvIII, preferably human EGFRvIII; or MSLN, preferably human MSLN. In some embodiments, such conjugate comprises (e.g., further comprises) a short peptide (e.g., a CKP or CKP variant) that binds a target listed above. In some embodiments, the Fab and the short peptide bind to the same target. In some embodiments, the Fab and the short peptide bind to different targets. In some embodiments, the Fab and the short peptide exhibit complementary or synergistic therapeutic activities.

Gastric cancer, breast cancer and/or cervical cancer may be targeted with a conjugate comprising one or more antigen-binding polypeptides (e.g., Fabs) that bind HER2, preferably human HER2. Gastric cancer and/or lung cancer may be targeted with a conjugate comprising one or more antigen-binding polypeptides (e.g., Fabs) that bind HER3, preferably human HER3. B-cell lymphoma and/or T cell lymphoma may be targeted with a conjugate comprising one or more antigen-binding polypeptides (e.g., Fabs) that bind CD20, preferably human CD20 and/or CD22, preferably human CD22. Myeloid leukemia may be targeted with a conjugate comprising one or more antigen-binding polypeptides (e.g., Fabs) that bind CD33, preferably human CD33. Ovarian cancer, lung cancer, breast cancer and/or gastrointestinal cancer may be targeted with a conjugate comprising one or more antigen-binding polypeptides (e.g., Fabs) that bind CA12-5, preferably human CA12-5. Gastrointestinal cancer, leukemia and/or nasopharyngeal carcinoma may be targeted with a conjugate comprising one or more antigen-binding polypeptides (e.g., Fabs) that bind HLA-DR, preferably human HLA-DR. In some embodiments, such conjugate comprises (e.g., further comprises) a short peptide (e.g., a CKP or CKP variant) that binds a target listed above. In some embodiments, the Fab and the short peptide bind to the same target. In some embodiments, the Fab and the short peptide bind to different targets. In some embodiments, the Fab and the short peptide exhibit complementary or synergistic therapeutic activities.

Colon cancer, breast cancer, ovarian cancer, lung cancer and/or pancreatic cancer may be targeted with a conjugate comprising one or more antigen-binding polypeptides (e.g., Fabs) that bind MUC-1, preferably human MUC-1. Colon cancer may be targeted with a conjugate comprising one or more antigen-binding polypeptides (e.g., Fabs) that bind A33, preferably human A33. Prostate cancer may be targeted with a conjugate comprising one or more antigen-binding polypeptides (e.g., Fabs) that bind PSMA, preferably human PSMA. Gastrointestinal cancer, pancreatic cancer, cholangiocellular cancer, lung cancer, breast cancer, ovarian cancer, skin cancer and/or oral cancer may be targeted with a conjugate comprising one or more antigen-binding polypeptides (e.g., Fabs) that bind the transferrin receptor, preferably the human transferring receptor. Pancreatic cancer, lung cancer and/or breast cancer may be targeted with a conjugate comprising one or more antigen-binding polypeptides (e.g., Fabs) that bind the transferrin receptor, preferably the human transferring receptor. Renal cancer may be targeted with a conjugate comprising one or more antigen-binding polypeptides (e.g., Fabs) that bind CA-IX, preferably human CA-IX. In some embodiments, such conjugate comprises (e.g., further comprises) a short peptide (e.g., a CKP or CKP variant) that binds a target listed above. In some embodiments, the Fab and the short peptide bind to the same target. In some embodiments, the Fab and the short peptide bind to different targets. In some embodiments, the Fab and the short peptide exhibit complementary or synergistic therapeutic activities.

The therapeutic agent for delivery to target tissues, e.g., as provided above, may be a cytotoxic or cytostatic agent, which kills or inhibits tumor cells (Syrigos and Epenetos, Anticancer Research 19:605-614 (1999); Niculescu-Duvaz and Springer, Adv. Drg. Del. Rev. 26:151-172 (1997); U.S. Pat. No. 4,975,278), whereas systemic administration of these agents may result in unacceptable levels of toxicity to normal cells as well as the tumor cells sought to be eliminated. The cytotoxic agent may be one or more selected from daunomycin, doxorubicin, methotrexate, and vindesine, radionuclides, bacterial toxins such as diphtheria toxin, plant toxins such as ricin, small molecule toxins such as geldanamycin, maytansinoids, and calicheamicin. The toxins may affect their cytotoxic and cytostatic effects by mechanisms including tubulin binding, DNA binding, or topoisomerase inhibition.

Cytotoxic or cytostatic agents may also include enzymatically active toxins and fragments thereof including diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232. Examples of radionuclides that can be used include ²¹²Bi, ¹²⁵I, ¹³¹I, ¹³¹In, ⁹⁰Y, ²¹At, ¹⁸⁶Re, ¹⁸⁸Re, ¹⁵³Sm, ³²P, ²¹²Pb, radioactive isotopes of Lu, and the like.

In a specific example, the conjugate of the invention comprises a first antigen-binding polypeptide (e.g., a Fab) that binds CEA; and a second antigen-binding polypeptide (e.g., a second Fab) that binds an indium-111-labeled peptide, e.g., for pre-targeted delivery of indium-111. In another specific example, the conjugate of the invention comprises a first antigen-binding polypeptide (e.g., a Fab) that binds CD38; and a second antigen-binding polypeptide (e.g., a second Fab) that binds a ⁹⁰Y complex, for pre-targeted delivery of ⁹⁰Y. In still another specific example, the conjugate of the invention comprises a first antigen-binding polypeptide (e.g., a Fab) that binds CD22; and a second antigen-binding polypeptide (e.g., a second Fab) that binds CD19; and further comprises diphtheria directly attached to the first and/or second antigen-binding polypeptides. In some embodiments, such conjugate comprises (e.g., further comprises) a short peptide (e.g., a CKP or CKP variant). In some embodiments, the Fab and the short peptide bind to the same target. In some embodiments, the Fab and the short peptide bind to different targets. In some embodiments, the Fab and the short peptide exhibit complementary or synergistic therapeutic activities.

In some embodiments, the cytotoxic agent is attached to one or more of the antigen-biding polypeptide (or, if present, one or more short peptides) of conjugates of the invention, e.g., using known protein-coupling agents. Example of protein-coupling agents include, without limitation, N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (pazidobenzoyl) hexanediamine), bis-diazonium derivatives (such as bis-(pdiazoniumbenzoyl)-ethylenediamine), diisocyanates (such as toluene 2,6-diisocyanate), and bis-active fluorine compounds (such as 1,5-difluoro-2,4-dinitrobenzene). In one embodiment, carbon-14-labeled 1-isothiocyanatobenzyl-3-methyl-diethylene triaminepentaacetic acid (MX-DTPA) is an exemplary chelating agent for conjugation of radionuclide to an antigen-binding polypeptide. See, e.g., WO 94/11026.

In particular embodiments, the cytotoxic agent is selected from the group consisting of calicheamicin, maytansinoids, dolastatins, aurostatins, a trichothecene, and CC1065, and the derivatives of these toxins that have toxin activity. Other antitumor agents that can be used in the conjugates of the present invention include BCNU, streptozotocin, vincristine and 5-fluorouracil, the family of agents known collectively LL-E33288 complex described in U.S. Pat. Nos. 5,053,394 and 5,770,710, as well as esperamicins (U.S. Pat. No. 5,877,296).

In some embodiments, the therapeutic agent is a nucleotide, e.g., siRNA, interfering RNA, a CRISPR RNA, an shRNA, an aptamer, or a ribozyme.

b. Delivery of Diagnostic Agents

A conjugate of the invention (e.g., a peptide-NLP conjugate, peptide-NLP-polypeptide conjugate, and/or NLP-polypeptide conjugate) may comprise a detectable agent, e.g. for use in diagnoses or basic research, for monitoring the progress of a disease and/or efficacy of a therapeutic agent, for identifying certain subpopulations of patients, e.g., patients more likely to respond to a therapeutic agent, and the like.

A detectable agent is a label that can be visualized or otherwise detected as being localized at cells or tissues to which the agent has been delivered, generally via a signal emitted from the label. The signal may be, e.g., radioactivity, fluorescence, chemiluminescence, production of a compound in outcome of an enzymatic reaction, and the like. Examples of detectable agents for use with conjugates of this invention include, without limitation, radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. In some embodiments, radioactive atoms are used as the detectable agent, such as for scintigraphic studies. Examples of radioactive atoms include, e.g., tc99m or I¹²³. In some embodiments, a spin label is used as the detectable agent, such as for nuclear magnetic resonance (NMR) imaging or magnetic resonance imaging (MRI)), examples of which include iodine-123 again, iodine-131, indium-111, fluorine-19, carbon-13, nitrogen-15, oxygen-17, gadolinium, manganese or iron.

In some embodiments, the detectable agent is a fluorophore. The term “fluorophore” refers to a substance or a portion thereof which is capable of exhibiting fluorescence in a detectable image. For example, in some embodiment, a membrane-lipid can be a fluorescently labeled, such as (1-Olcoyl-2-[12-[(7-nitro-2-1,3-benzoxadiazol-4-yl)amino]dodecanoyl]-sn-Glycero-3-Phosphocholine). One of skill in the art will recognize that, in such embodiments, the signal from a detectable agent can be enhanced by the amount of fluorescently labeled lipid used in assembling the NLP conjugates.

The detectable agent may be incorporated into the conjugate as described above, or in other ways known in the art. For example, an antigen-binding polypeptide (e.g., a Fab) may be biosynthesized or synthesized by chemical amino acid synthesis using amino acid precursors comprising, e.g., fluorine-19 in place of hydrogen. Detectable agents such as tc99m, ¹²³I, ¹⁸⁶Re, ¹⁸⁸Re, and ¹¹¹In can be attached via a cysteine residue in the peptide. Yttrium-90 can be attached via a lysine residue. The IODOGEN method (Fraker et al. Biochem. Biophys. Res. Commun. 80:49-57 (1978)) can be used to incorporate iodine-123. “Monoclonal Antibodies in Immunoscintigraphy” (Chatal, CRC Press 1989) describes other methods in detail.

Additionally or alternatively, such modifications can be made to the short peptide, wherein the conjugate comprises a short peptide.

c. Delivery across the Blood-Brain Barrier

Another aspect of the present invention relates to methods of delivering a biologically active agent or a detectable agent to the brain or central nervous system, for example in treating neurological disease. Treatment modalities targeting the brain and CNS face particular challenges, in particular the blood-brain barrier (BBB) that regulates molecular trafficking and may keep drugs from reaching the brain parenchyma. Accordingly, the ability of drugs to cross the BBB is of paramount importance in pharmacological treatment of brain diseases (Dal Margo, et al., “Artificial apolipoprotein corona enables nanoparticle brain targeting” Nanomedicine: Nanotechnology, Biology, and Medicine, 14 (2018) 429-438).

In some embodiments, the present invention provides conjugates, pharmaceutical compositions, formulation, methods, systems and kits, for delivering a biologically active agent or a detectable agent to the brain or central nervous system. Generally, a conjugate of the present invention is administered systemically to an individual, e.g., by an intravenous route, allowing the conjugate to circulate and eventually translocate the BBB. For example, a liquid formulation may be used, comprising a pharmaceutically acceptable vehicle and a conjugate of an antigen-binding polypeptide (e.g., a Fab or Fab-like molecule), a nanolipoprotein particle, and the biologically active agent or detectable agent. In some embodiments, the conjugate further comprises a short peptide (e.g., a CKP or a CKP variant). The formulation may be administered by intravenous injection, or other appropriate systemic or local route.

For brain/CNS delivery, the nanolipoprotein of the conjugate will comprises a scaffold protein that allows translocation of the conjugate across the BBB. In some embodiments, the scaffold protein will be an apolipoprotein, in particular, apoE4, apoE2, a truncated version of either, or a combination of one or more of apoE2, a truncated version of apoE2, apoE4, and a truncated version of apoE4. ApoE, for example, has been shown to play a prominent role in delivery of nanoparticle-bound drugs across the BBB (Dal Margo, et al., “Artificial apolipoprotein corona enables nanoparticle brain targeting” Nanomedicine: Nanotechnology, Biology, and Medicine, 14 (2018) 429-438). In some embodiments, the pharmaceutical composition further comprises a surfactant, e.g., polysorbate 80. The conjugates may show increased translocation across the BBB and increased (e.g., significantly increased) delivery of the biologically active agent or detectable agent to the brain or central nervous system, compared the case when not bound to a conjugate of the invention (e.g., when not associated with/covalently bound to a NLP-Fab conjugate or an NLP-Fab-short peptide conjugate).

Neurological disease that may be treated according to the invention include any disease of the central and peripheral nervous systems. Non-limiting examples include diseases of the brain, spinal cord, cranial nerves, peripheral nerves, nerve roots, autonomic nervous system, neuromuscular junction, and muscles. In particular embodiments, conjugates of the present invention may be used in treating neurological diseases of the brain and central nervous system.

In specific examples, conjugates of the present invention find used in treating, e.g., Alzheimer disease, other dementias, brain tumors, cerebrovascular disease, epilepsy, migraine, other headache disorders, multiple sclerosis, neuro-infections, neurological disorders due to malnutrition, Parkinson's disease, stroke, traumatic disorders of the nervous system due to head trauma, and the like.

### VIII. Systems and Kits

Another aspect of the present invention relates to systems and/or kits, such as systems and kits designed to facilitate performing one or more of the processes or methods of use described herein. Generally, the system or kit comprises a conjugate (e.g., an peptide-NLP conjugate, peptide-NLP-polypeptide conjugate, and/or NLP-polypeptide conjugate), pharmaceutical composition, or formulation of the invention, and/or related components, where the conjugate, composition, and components are provided in different compartments or containers. Suitable containers include, for example, bottles, vials (e.g. dual chamber vials), syringes (such as dual chamber syringes) and test tubes. The container may be formed from a variety of materials such as glass or plastic. A compartment refers to a division within a container, e.g., to keep separate different components within a given container, and may comprise the same or different materials as the container.

In some embodiments, the membrane-forming lipids, scaffold protein, antigen-binding polypeptide (e.g., a Fab), and in some embodiments, a peptide-conjugated C₄₋₂₈ fatty-acyl are provided in one or more different compartments or containers of a kit, along with instructions for preparing a conjugate of the invention. Instructions may be included in a package insert. The package insert may also contain information about the indications, usage, dosage, administration, combination therapy, contraindications, and/or warnings concerning the conjugate of the invention, e.g., for use in therapeutic, prophylactic, or diagnostic indications.

In some embodiments, the NLP and antigen-binding polypeptides (e.g., Fabs), and in some embodiments, peptide-conjugated C₄₋₂₈ fatty-acyls, are provided in separate compartments or containers of a kit. In some embodiments, the kit provides peptide-NLP conjugates, peptide-NLP-polypeptide conjugates, and/or NLP-polypeptide conjugates and a pharmaceutically acceptable carrier in separate compartments or containers of a kit, for preparing a pharmaceutical composition, as described herein. For example, the kit may include instructions for making low viscosity formulations of the conjugates, e.g., for subcutaneous or ocular delivery of high concentrations of the conjugates in low volumes. In some embodiments, the conjugates further comprise an additional therapeutic agent or detectable agent (label), or the kit provides the additional therapeutic agent and/or the detectable agent (label) in one or more further compartments or containers, optionally with instructions for use with the conjugates.

In some embodiments, conjugates of the invention are provided in a lyophilized formulation in a kit, optionally including instructions for reconstituting the material and/or use. For example, a container may hold the lyophilized formulation and have a label on, or associated with, the container that indicates directions for reconstitution and/or use. The label may indicate that the lyophilized formulation is reconstituted to conjugate concentrations, as described above. The label may further indicate that the formulation is useful or intended for subcutaneous or ocular administration. The container holding the formulation may be a multi-use vial, which allows for repeat administrations (e.g. from 2-6 administrations) of the reconstituted formulation. The kit may further comprise a second container or compartment comprising a suitable diluent (e.g. BWFI). The kit may further include other materials desirable from a commercial and user standpoint, including other buffers, diluents, filters, needles, syringes, and package inserts with instructions for use.

### IX. Methods of Increasing the Biological Activity of a Short Peptide Attached to the Surface of a Nanoparticle

Also provided herein is a method of increasing the activity (e.g., biological activity) of a short peptide (e.g., a peptide between 20-60 amino acids in length) attached to the surface of a lipid-based nanoparticle. In some embodiments, the method comprises providing a lipid-based nanoparticle comprising a short peptide attached (e.g., covalently attached) to the surface of the nanoparticle, wherein one or more lipids of the nanoparticle presents a functionalized group, and allowing the nanoparticle to contact a polypeptide (e.g., a Fab) having a functional group under conditions that favor conjugation of said functionalized group to said functional group. In some embodiments, the method further comprises purifying the conjugate comprising the lipid-based nanoparticle, the short peptide, and the polypeptide. In some embodiments, the nanoparticle is a liposome. In some embodiments, the nanoparticle comprises a lipid bilayer. In some embodiments, the lipid-based nanoparticle is a solid lipid nanoparticle (SLN). In some embodiments, the lipid-based nanoparticle is a nanostructured lipid carrier (NLC). In some embodiments, a spacer connects said functionalized group to a lipid on the surface of the lipid-based nanoparticle. In some embodiments, a spacer connects said complementary functional group to said polypeptide. Exemplary functionalized groups and complementary functional groups for conjugating the polypeptide to the lipid are described in detail elsewhere herein. In some embodiments, the polypeptide is an antigen-binding polypeptide. Exemplary antigen-binding polypeptides for conjugation to a lipid in a liposome or nanoparticle comprising a lipid bilayer are described in detail elsewhere herein. In some embodiments, the peptide is a CKP. In some embodiments, the peptide is a CKP variant. In some embodiments, conjugation of the polypeptide to the nanoparticle increases the activity (e.g., biological activity) of the short peptide by any one of about 2-, 3-, 4-, 5-, 6-, 7-, 8-, 9-, 10-, 20-, 50-, 100-, or 150-fold as compared to the activity (e.g., biological activity) of the short peptide on a lipid-based nanoparticle without the polypeptide.

All references, publications, and patent applications, e.g., disclosed herein, are hereby incorporated by reference in each of their entireties.

### EXAMPLES

The following are examples of methods and compositions of the invention. It is understood that various other embodiments may be practiced, given the general description provided above.

### Example 1: Production of NLP-Fab Conjugates

### Materials

1,2-dioleoyl-sn-glycero-3-phosphocholine (DOPC) and DOPE-MCC were purchased from Avanti Polar Lipids (Alabaster, AL). Human serum, Alexa Fluor 488 NHS Ester (AF488) were obtained from Thermo Fisher (Carlsbad, CA). All other reagents were ordered from Sigma-Aldrich (St. Louis, MO).

Protein Expression and Purification of apoE422k and Fabs

ApoE422k was produced in E. coli cells under shake flask conditions using established expression plasmids and methods (46, 47). Once harvested, cells were ruptured, spun down and supernatant collected. ApoE422k was purified over a Ni-NTA column (XK16/20 3 ml) followed by SEC (Superdex 75 16/60). For the Ni-NTA purification, the column was washed and protein bound in 50 mM Phosphate buffer, 200 mM NaCl, 10 mM imidazole, pH 8 (Buffer A). The protein was washed extensively (20 column volumes) with Buffer A+0.2% Triton X114+0.2% Triton X100 and eluted with 50 mM Phosphate buffer, 200 mM NaCl, 400 mM imidazole, pH 8. The pooled fractions were filtered and concentrated with a 3 kDa molecular weight cutoff spin concentrators. The His tag was then removed through Tobacco Etch Virus nuclear-inclusion-a endopeptidase (TEV protease) digestion (TEV tag was added on N-terminus between the His tag and protein sequence). The TEV protease was added to the purified protein at a protein weight to TEV weight ratio of 100 to 1. The cleaved protein was purified from the TEV protease, which contained a His tag, by passing the reaction mixture over a Ni-NTA column (XK 16/20 3 ml). The pooled protein was concentration and run on SEC (Superdex 75 16/60) in PBS. SEC Fractions were collected and analyzed for identity using mass spectrometry and for aggregation by size-exclusion chromatography (SEC). Fractions with the correct molecular weight (MW) and aggregate level <5% were pooled and protein concentration was determined by absorbance at 280.

In this experiment, Fab constructs were designed with a C-terminal cysteine (referred to as Fab for simplicity) to enable site specific conjugation to NLP. Experiments to evaluate Fab conjugation and effect of conjugation on Fab-NLP stability were performed using a species matched non-targeting Rabbit Fab, specific to an irrelevant cytosolic antigen (cMET). Expression and purification of Rabbit Fab was performed as previously described (47, 48). Experiments to evaluate Fab activity after conjugation to the NLP platform were performed with human anti-OX40 Fab and human anti-factor D (AFD) Fab, which were purified as described previously (45). All purified Fabs were de-cysteinylated using 20 mM DTT to reduce cysteine conjugates and re-oxidized with 6.5 mM glutathione (GSH). Samples were then buffer exchanged and washed and stored in 200 mM Arginine Succinate pH 5 to limit further cysteinylation.

### NLP Assembly

NLPs were assembled according to a previously reported procedure (20). For all NLP assemblies, the total lipid to apoE422k molar ratio was 80:1, which was previously shown to result in a relatively homogeneous NLP population (16, 19, 31, 46). NLPs were assembled with a combination of DOPC and DOPE-MCC lipids as described in the results section. These lipids were either prepared or obtained in chloroform and aliquoted into glass vials. Chloroform was then removed using a stream of N2 under agitation to form a thin lipid film. Lipids were solubilized in either PBS buffer (137 mM sodium chloride, 2.7 mM potassium chloride, 10 mM phosphate buffer, pH 7.4) or 50 mM Na Phosphate buffer, pH 6.0, 150 mM NaCl using 80 mM sodium cholate. After addition of apoE422k (150 μM in final assembly volume), samples were incubated at 22° C. for at least 1 hour. For cholate removal, the samples were incubated with detergent removing biobeads (Sigma-Aldrich) for 2 h with rocking in 500 μL costar 0.22 spin filters. After two hours of rocking, the sample was centrifuged at 200 g for 5 min and the filtrate containing the NLPs was collected. Samples that were not going to subsequently be used for Fab conjugation were purified over SEC using an AKTA Avant system and S200 10/300 Increase column. In contrast, samples that were going to be conjugated to Fabs were not purified at this stage but rather immediately conjugated as described below.

### Fab-NLP Conjugation and Purification

After the dialysis step described above, apoE422k concentration in NLPs containing DOPE-MCC was determined and the NLPs were incubated with Fab in 50 mM Na Phosphate buffer, pH 6.0, 150 mM NaCl at Fab:NLP molar ratios ranging from 0-60. Conjugation was performed the same day the NLPs were assembled to limit hydrolysis of the maleimides. The sample was placed on a rocker and incubated for 2-4 hours. After the 2-4 hour reaction incubation period, n-acetylcysteine (NAC) was added at a 2-fold molar access over DOPE-MCC to quench any unreacted maleimide. The resulting Fab-NLP conjugates were purified using an AKTA Avant system and S200 10/300 Increase column. Each fraction across the Fab-NLP peak was analyzed by SEC-MALS as described below and fractions were pooled based on MW and hydrodynamic radius (Rₕ) analysis to yield a homogeneous Fab-NLP sample.

### Results and Discussion for Example 1

### Optimizing Fab-NLP Conjugation Conditions

This study established the suitability of utilizing the NLP as a Fab delivery vehicle. FIG. 1A shows a schematic overview of the strategy developed for generating Fab-NLP conjugates. Broadly, this approach involved assembling NLPs with a bilayer-forming lipid (helper lipid) and functionalized lipids (lipids bearing a functionalized head group) for conjugation with a Fab molecule containing an orthogonal reactive group. In these experiments, DOPC was selected as the helper lipid because it was previously shown to form more stable particles relative to DMPC containing NLPs (20).

An important consideration in selecting the appropriate functionalized lipid for conjugating Fabs to the NLP was the choice of a chemoselective reaction pair to avoid reactivity with native functional groups present on the apoE422k scaffold protein (e.g. amino and carboxyl groups). Since the apoE422k protein contains no cysteines (thiol reactive groups), we selected thiol-maleimide chemistry employing a lipid containing a maleimide head group, DOPE-MCC, for Fab conjugation. Site-specific conjugation of the Fab was realized by retaining Cys-227, a residue normally involved in the interchain hinge disulfide bond in a full-length IgG, as the Fab C-terminal residue (referred to as Fab for simplicity in the Examples). The resultant Fab contained this unpaired Cys with a free, reactive thiol (48). This strategy allowed for assembly of the NLPs with maleimide reactive groups decorating the NLP surface followed by site-specific conjugation to thiol reactive Fabs (FIG. 1A).

### Example 2: Analysis of NLP-Fab Conjugates

### SEC-MALS/QELS Analysis of NLP and Fab-NLP Conjugates

MW and Rₕ were determined using an Acclaim SEC-1000 analytical SEC column (Thermo Fisher Scientific), with isocratic gradient of phosphate buffered saline (PBS) (with an additional 150 mM NaCl spike in), coupled to a multi-angle light scattering system (MALS) (Wyatt Instruments). In addition, diffusion coefficients (D) were measured using quasi elastic light scattering (QELS) where fluctuations in intensity of laser light scattered were captured using a single photon counting module detecting at a 99.0° angle. Assuming a spherical shape, the Stokes-Einstein relationship was used to calculate Rₕ from D.

### LCMS Analysis of NLP and Fab-NLP Conjugates

LCMS analysis of the NLP and NLP-Fab conjugate was performed using an Agilent 6230 ESI-TOF LC/MS. A Kinetex 2.6 μm XB-C18 column (Phenomenex), heated to 80° C., was used to analyze the injected NLP samples. The solvent was run as a gradient from a mixture of 30% methanol and 70% water to 100% 2-propanol. All solvents had 0.05% trifluoroacetic acid. ApoE422k and Fab alone were injected to provide baseline masses.

### HPLC Analysis of NLP, Fab-NLP and Fab Loading

ApoE422k and Fab concentration in the Fab-NLP conjugate were analyzed using a combination of HPLC analysis and absorbance at 280 nm. The apoE422k concentration was determined on an Agilent 1290 Infinity Bio-inert HPLC using the same column and gradient described above in the LCMS analysis. A sample A280 HPLC chromatogram of the NLP-Fab conjugate is shown in FIG. 7A. Two peaks were observed corresponding to apoE422k (4.95 min) and Fab conjugated to DOPE-MCC (˜5.15 min). The apoE422k concentration was determined based on standard curves generated by injecting 1-8 μg of apoE422k and integrating the area under the curve (FIG. 7B). Given the challenges with generating a Fab-DOPE-MCC reagent, which was primarily due to incompatible solubility of lipids and proteins, apoE422k concentration and A280 total absorbance was used to quantify the Fab-DOPE-MCC conjugate rather than an HPLC standard curve using the following equation:

[Fab]=(Abs280−[apoE422k]·ε_{apoE422k})/ε_{Fab}

Where [Fab], Abs280, [apoE422k], ε_{apoE422k} and ε_{Fab} is the Fab concentration, absorbance at 280 nm, apoE422k concentration determined by HPLC analysis, extinction coefficient of apoE422k and extinction coefficient of the Fab, respectively.

### Results and Discussion for Example 2

### Further Optimizing Fab-NLP Conjugation Conditions

To evaluate the impact of incorporating maleimide-thiol reactivity, the effect of DOPE-MCC on NLP assembly was assessed. NLPs were assembled at a range of DOPE-MCC concentrations (0, 10, 20 and 30 mol %) in PBS pH 7.4 and analyzed by SEC-MALS/QELS (FIGS. 1B and 8A). The resulting SEC chromatograms across the different DOPE-MCC concentrations (FIG. 8A), along with measured MWs and Rₕs (FIG. 1B; black dots MW, gray squares Rₕ), all yielded nearly identical results indicating that the functional lipid did not have a considerable impact on overall NLP shape and size. However, further analysis by LCMS revealed product complexity (FIGS. 1C-1D) not captured by the native analysis. Whereas a single TIC peak at an elution time of ˜4.9 min was observed for apoE422k alone (FIG. 1C circles), with deconvoluted mass (˜22 kDa) (FIG. 1D circles) in-line with the expected mass of apoE422k, no dominant apoE4222k peak was observed for the NLP assembled in PBS pH 7.4. Instead, multiple TIC peaks with retention times ranging from 4.8-6.5 min (FIG. 1C plain line), were present at all MCC concentrations (data not shown), where a pattern of three major peaks at ˜23 kDa, 24 kDa and 25 kDa were observed upon deconvolution (FIG. 1D plain line).

Interestingly, these mass additions correspond to the MW of DOPE-MCC lipid (960 kDa), suggesting that during the assembly and purification process in PBS buffer at pH 7.4, DOPE-MCC was conjugating to and lapidating the apoE422k scaffold protein and the 23 kDa, 24 kDa and 25 kDa correspond to apoE422k conjugated to 1, 2 and 3 lipids, respectively. Although maleimide reactivity for thiols is highly specific at pH values between 6.5-7.5, there have been reports of reactivity with amino groups on proteins at alkaline pH values (50). The high concentration of maleimides in close proximity to exposed lysine residues, as well as the absence of sulfhydryls at this step, increases the opportunity for this side reaction to proceed as has been observed previously (51). To address this liability, the reaction was performed at pH 6.0. These conditions are less optimal for maleimide-thiol chemistry and requires an extended reaction time for Fab conjugation, but reduces the likelihood of unprotonated, nucleophilic amines. Assembly at pH 6.0 may also help to minimize maleimide hydrolysis, which occurs faster at higher pH values and results in an irreversible and unreactive maleic acid (52). Thusly, NLP were assembled with 20 mol % DOPE-MCC in 50 mM Na Phosphate buffer, 150 mM NaCl at pH 6.0 and analyzed by LCMS following SEC purification. Using these assembly conditions, only a single peak in the LC chromatogram (FIG. 1C triangles) was observed and this peak deconvoluted to the expected mass of apoE422k (˜22 kDa) (FIG. 1D triangles). These combined findings strongly suggest that DOPE-MCC lipids were conjugating to exposed lysines on apoE422k.

Interestingly, no aggregation was present in any of the assemblies where significant apoE422k-DOPE-MCC conjugation was detected (FIG. 8A), suggesting that crosslinking may be an intraparticle event rather than interparticle. To further assess the effect of apoE422k-DOPE-MCC conjugation on NLP aggregation, NLPs of identical composition (20 mol % DOPE-MCC) were prepared at pH 7.4 and 6.0 and analyzed by SEC-MALS. In line with previous observations, MW and Rₕ of both samples were identical (FIG. 8B), despite extensive apoE422k lipidation of the sample prepared at pH 7.4. These data provide further evidence that the DOPE-MCC:apoE422k conjugation is not resulting in interparticle crosslinking but rather intraparticle crosslinking.

We hypothesize that lysine residues residing on the scaffold protein interface with the lipid core experience a shift in their pKa rendering them deprotonated at pH 7.4 and able to react with adjacent maleimides. Of the 8 lysines in the apoE422k protein, 7 are located on the helical domains that encase the lipid bilayer (53) and are readily accessible to DOPE-MCC in the lipid bilayer. In addition, a decrease in lysine side chain pKa in hydrophobic environments has been previously reported with lysine residues both buried in proteins and DOPC bilayers (54, 55). In these studies, water inaccessible lysine residues exhibited depressed pKa values below the normal pKa≥10 to as low as 5.2 and 6, respectively. The selective decrease in pKa values of only lysine residues facing the NLP lipid bilayer may explain the lack of observed intermolecular aggregates as their nucleophilic amines are likely unable to interact with headgroups on nearby NLPs. The detection of apoE422K lipidation by DOPE-MCC underscores the importance of using advanced analytical techniques such as LCMS to evaluate new therapeutic delivery platforms. Early detection of process-related impurities and identification of platform concerns facilitates successful development of novel platform technology. This crosslinking event would not have been detected using traditional analytical SEC methods, and while it is unclear if this side-product results in meaningful differences in stability or activity, the variability cannot be controlled and could otherwise cause manufacturing difficulties.

### Fab-NLP Conjugate Purification and Fab Loading

Once assembly conditions were established, with minimal to no DOPE-MCC:apoE422k crosslinking and a relatively homogeneous NLP population, Fab-NLP conjugation methods were further optimized. A Rabbit Fab molecule that was specific for an irrelevant cytosolic target (cMET) was used as a surrogate reagent for these experiments. Production and purification of Fab-NLP conjugates were performed using sequential NLP assembly and conjugation steps, with Fab addition happening directly after biobead-based cholate removal from the NLP, without a NLP purification step. This protocol was selected to minimize the potential for maleimide hydrolysis prior to conjugation. 20 mol % DOPE-MCC was chosen for scale up based on previous studies that reported maximal binding efficiency for functional lipid compositions ranging from 20-35 mol % (31). Purification protocols were initially developed at a Fab:NLP molar reaction ratio of 20. Fabs were incubated with the NLP for 2-3 hours and NAC was added at 2-fold molar concentration over DOPE-MCC to quench unreacted maleimides and prevent DOPE-MCC:apoE422k crosslinking.

The Fab-NLPs were then purified over an S200 SEC column and three main peaks were observed in the SEC profile; NLP-Fab conjugate (rₜ˜10 min), Fab dimer (rₜ˜14 min), unconjugated Fab (rₜ˜16 min) (FIG. 2A). The appearance of the Fab dimer was caused by dimerization via disulfide formation at the C-terminal thiols of the Fabs during the conjugation step. Interestingly, the starting material did not contain any Fab dimer (data not shown) so this appears to have occurred during the conjugation step and was not consistently observed across other Fabs, suggesting that there might be unique properties (local PI) inducing this phenomenon. Regardless, we were still able to demonstrate successful conjugation of Fab to the NLP.

In depth analysis by SEC-MALS across the NLP peak guided the pooling strategy (FIG. 2A inset). In general, the NLP peak was relatively symmetrical and Rₕ values varied only slightly across the peak with the largest variation (12.9-10.5 nm) at the front end of the peak (9-9.5 min retention time) and the smallest variation (10.3-9.6 nm) across the middle and end of the peak (9.5-10.25 min retention time) (FIG. 2A inset). Therefore, to generate the most homogeneous NLP-Fab formulation, sample fractions between 9.5-10.25 min were pooled and subject to LCMS analysis. LCMS analysis of Fab alone yielded a single peak in the TIC chromatogram with a retention time of ˜5.4 min (FIG. 2B circles). The deconvoluted mass of this peak was ˜45.7 kDa, which corresponds to the expected mass of the Fab. Two peaks were observed in the TIC chromatogram of the NLP-Fab conjugate pool within this retention time (FIG. 2B triangles) where the deconvoluted mass of these peaks corresponded to the DOPE-MCC:Fab conjugate (˜46.7 kDa) and apoE422k (FIG. 2C triangles). Combined, these results suggest the Fab was successfully conjugated to the NLP via the DOPE-MCC lipid.

To further explore the Fab loading capacity of the NLP platform, NLPs were conjugated with increasing Fab concentrations and the number of Fabs per NLP was measured following a purification step, as described in the materials and methods. In these experiments, the Fab reaction concentration was varied from 0-270 μM, which corresponds to 0-50 Fabs/NLP. As shown in FIG. 3A, the NLP peak gradually increased with an increase in Fab concentration up to a saturation level of 216 μM. A gradual increase in the Fab dimer and unconjugated Fab peak was also observed up to the saturation point of 216 μM, followed by a dramatic increase at higher concentration (270 μM), indicating that conjugation to the NLP was saturated and all additional reagent added beyond 216 μM was not conjugating to the NLP.

The amount of Fab conjugated under each condition was quantified as described above (FIG. 3B). At lower Fab concentrations (0-200 μM), a linear increase in Fab loading from 0-30 Fab/NLP was observed. However, this trend flattened out at higher Fab concentrations (>200 μM) and no additional Fab could be loaded onto the NLP beyond ˜30 Fabs/NLP. Based on published crystal structures, the cross-sectional diameter of a typical Fab is ˜4 nm (56), which corresponds to a surface area ˜12.6 nm², while based on our SEC-QELS analysis, the Rₕ of the NLP was determined to be ˜7.5 nm, corresponding to a total binding surface area of 350 nm², resulting in ˜30 Fabs/NLP. This maximal loading capacity corroborates our results and is consistent with the theoretical maximal loading capacity when considering the dimension of the Fab and NLP binding surface.

To further characterize the effect of Fab loading on NLP structure, the MW and Rₕ of Fab-NLP conjugates were measured as a function of Fab loading. Based on the MALS analysis across the Fab-NLP conjugate peak, the MW of the Fab-NLP conjugate increased at higher Fab loading, where the MW increased from ˜225 kDa at 0 Fab loading up to ˜900 kDa at a Fab:NLP ratio of 30 (FIG. 3C). Interestingly, this increase in MW implies a lower loading capacity of ˜16 Fabs/NLP compared with the HPLC analysis. This discrepancy may be due to a change in the dn/dc values for the Fab-NLP conjugate relative to free Fab, which could have affected the calculations. Given that the HPLC method is an independent measure of apoE422k and Fab concentrations, we believe these values are a more accurate reflection of the Fab loading.

Despite this discrepancy, the MALS analysis clearly indicates that there is a linear increase in MW with Fab loading as expected. In contrast to the MW analysis, the Rₕ did not change appreciably with increasing Fab loading (FIG. 3D). These results are consistent with the discoidal nature of the NLPs and indicate that NLP surface area is the dominant factor controlling Fab valency. This phenomenon has been observed in two previous studies where globular proteins were conjugated to NLPs (20, 31). In both examples, His-tagged proteins were attached to NLPs containing nickel-chelating lipids and the maximum loading achieved were at numbers consistent with the theoretical binding capacity based on the protein size and NLP surface area. It is worth noting that the Rh analysis assumes a spherical shape, which may not be completely accurate for these presumed discoidal nanoparticles. However, it was previously reported that NLP shape in solution is dynamic and NLPs can adopt a variety of different conformations beyond a static discoidal shape that would be more reflective of a spherical shaped particle (57); thus, we are reasonably confident in the MALS analysis described above.

### Example 3: Manufacturability of NLP-Fab Conjugates

### Rheology Analysis of Fab-NLP Conjugate

Viscosity was measured using the Anton Paar Physica MCR 501 rotary rheometer, with the CP20-0.5° cone and plate configuration. The CP20-0.5 geometry has a diameter of 20-mm and an angle of 0.5°. The measurements were performed at 25° C. using the rheometer temperature controller (Peltier plate with circulating fluid from water bath). Approximately 20 μL of each sample was loaded onto the bottom plate for measurement, after which the cone was lowered slowly to the desired gap width. The measured torque determined the shear stress, from which the viscosity was calculated, as previously described.

### Lyophilization of NLP and Fab-NLP Conjugate

Fab-NLP conjugates were lyophilized using 80 mM trehalose as an excipient. This is a commonly used excipient for both protein and liposome formulations. In these experiments, Fab-NLP conjugates were purified in PBS. Trehalose was added to a final concentration of 80 mM. The Fab-NLP protein concentration prior to freezing was 2 mg/ml. 300 μL of the Fab-NLP sample was frozen by incubation on dry ice for 60 min. The samples were then lyophilized on a Labconco lyophilizer overnight. After lyophilization was complete, samples were reconstituted in water and analyzed by SEC.

### Results and Discussion for Example 3

### Fab-NLP Formulation

A known challenge with the production of nanoparticle-protein conjugates is formulation during manufacturing. In particular, the relationship between protein concentration and viscosity can have a significant impact on critical parameters for drug manufacturing. This becomes particularly important when high concentration drug or protein formulations are required due to volume restrictions, such as with ocular delivery and sub-cutaneous administration where injection volume is limited to 100 μl (58) and 1-2 ml (59, 60), respectively. At high protein concentrations, protein-protein interactions may lead to increases in viscosity that can be exacerbated by the presence of other conjugated moieties due to an increase in the excluded volume (61). Therefore, to assess the effect of NLP based delivery on viscosity, the relationship between Fab concentration and viscosity was compared between the naked Fab, Fab-NLP conjugate and a Fab-PEG conjugate (47) (FIG. 4A). These experiments were performed using a biologically active human anti-factor D Fab for improved translatability. To permit better direct comparison in terms of Fab valency, a commercially available PEG scaffold was selected, where the scaffold had eight PEG arms containing maleimide-reactive termini that allowed for conjugation of eight Fabs/PEG. The details of generating and characterizing the eight arm PEG conjugate have been described elsewhere (47). For the Fab-NLP conjugate, the Fab loading was ˜30 Fab/NLP, which was selected to maximize the Fab density on the NLP.

In these experiments, the viscosity of Fab alone remained relatively constant across the concentration range tested (FIG. 4A circles). In contrast, the viscosity profile of the Fab-PEG conjugate abruptly increased to ˜1000 cP when Fab concentration reached between 80-100 mg/ml (FIG. 4A triangles). Though a similar profile was observed for the Fab-NLP conjugate, the Fab concentration at which an exponential increase in viscosity occurred was much higher (>300 mg/ml) (FIG. 4A squares). These results indicate that much higher Fab formulation concentrations can be achieved using the NLP platform relative to the more traditional PEG-based approach. These findings were surprising, given that Fab constitutes only ˜30% of the overall drug product by mass in the NLP formulation, whereas the Fab content for the Fab-PEG conjugate is ˜90% by mass. One possible reason for this observed phenomenon is ordering of Fabs on the NLP surface. Since viscosity at high protein concentrations is driven by intramolecular protein interactions and the protein's ability to pack, it is plausible that conjugation of the Fabs at high density to the NLP surface induces an ordering not achievable in the PEG octamer format.

In addition to being compatible with high concentration formulation, Fab-NLP conjugate displayed robust behavior upon lyophilization. In these experiments, Fab-NLP conjugates were generated at a Fab loading of ˜18 Fab/NLP, lyophilized in the presence of 80 mM trehalose at a concentration of 5 mg/ml and analyzed by SEC-MALS. Trehalose was selected as an excipient because it has been widely used for the lyophilization of liposomes as well as red blood cells (62, 63). As shown in FIG. 4B, MW and Rₕ prior and post lyophilization exhibited little change, supporting the notion that the Fab-NLP conjugate is highly stable. Stability upon lyophilization is a desired formulation property due to compatibility with established manufacturing processes, in addition to a prolonged shelf-life (64). These results are consistent with previous reports that trehalose is an excellent excipient for lipid-based nanoparticle formulation and confirms that Fab-NLP conjugates can be compatible with a manufacturing process that requires lyophilization based formulation.

### Example 4: Activity of NLP-Fab Conjugates

### Anti-Factor D Fab Blocking Activity Assay

Activities of Fab-NLP conjugates were determined with a Factor B cleavage TR-FRET assay as described previously (47, 49). The conjugate molarity was calculated using the Fab MW instead of the multimer MW given the expected 1:1 stoichiometry of Fab inhibiting Factor D.

### Anti-OX40 Fab Agonist Activity Assay

The OX40 agonist assay was performed as previously described (45). In short, OX40 overexpressing Jurkat cells engineered with an NFκB luciferase reporter were seeded at 80,000 cells/well in 20 μl RPMI 1640 media containing 10% FBS in a 384-well tissue culture plate (Corning Inc., cat #3985BC). Anti-OX40 formats were serially diluted in media at 4× concentration and 10 μl of the concentrated antibodies were added to each well. All well volumes were brought to 40 μl and incubated for 16-18 h in CO₂ incubator with 37° C., 5% Co₂. 40 μl Bright Glo (Promega cat #E2610) was then added to each well and incubated with shaking at room temperature for 10 min. Luminescence was detected using a Perkin Elmer Envision plate reader.

### Results and Discussion for Example 4

### Inhibitory and Agonist Activity of Fab-NLP Conjugates

Conjugation and immobilization of proteins to nanoparticles in some instances can negatively affect protein activity (65, 66). These negative effects can be attributed to a variety of different factors, including, but not limited to, conformational changes upon conjugation, loss of freedom of motion, and protein denaturation. For Fab-NLP conjugation, we aimed to mitigate these issues by using site-specific chemistry to prevent conjugation in random orientations and by placing the conjugation handle on the opposite end (C-terminal region of the heavy chain) of the active region of the Fab (variable domains of the heavy and light chains). However, this does not guarantee that conjugation of the Fab to the NLP will not have any impact on activity. Therefore, to assess the effect of Fab conjugation, the inhibitory activity of the anti-Factor D Fab was measured using a time-resolved fluorescence energy transfer (TR-FRET) assay of complement factor D-dependent Factor B cleavage. The maximal TR-FRET signal occurs when factor B remains intact in the absence of factor D activity. In this assay, we measured factor D inhibition by anti-factor D Fab alone, Fab-NLP conjugate, and the Fab-NLP conjugate that was lyophilized and reconstituted (FIG. 5A). No significant difference in inhibitory activity was observed across all three reagents, demonstrating that there was no loss in inhibition due to conjugation and lyophilization. These findings indicate that in general conjugation of Fab through a C-terminal cysteine tag will not have a significant impact on the activity of the CDR regions of the heavy and light chains.

In addition to evaluating the effect of conjugation on the inhibitory activity of Fab, we also assessed the potential of using this platform to enhance agonist Fab activity through higher valency. Several clinically valuable agonist pathways, including the TNF family members, require valencies beyond two for potent activity (41, 42). The mAb field has attempted to address this limitation using a variety of engineering strategies, including the development of triabodies, tetrabodies, pentabodies as well as self-assembling hexamer IgG1 (43-45). However, these engineering strategies have been reported to negatively impact PK and it is difficult to achieve valencies beyond 3-6 (43), which may not be sufficient to agonize/antagonize the pathway of interest.

Therefore, we next wanted to test the potential of using the Fab-NLP conjugate platform to agonize a pathway known to require higher valency. The OX40 TNF family receptor has been reported to require higher valency to induce agonist activity. In a recent publication, moderate agonizing activity was reported for a valency of 4 and highly potent activity was only observed for a hexameric IgG format, which had a valency of 12 (45). Therefore, the OX40 receptor was used as a model system to evaluate the effect of Fab-NLP valency on agonist activity. Fab-NLP conjugates were generated at OX40 Fab to NLP molar ratios ranging from 0 to 30 and the agonist activity was measured in a Jurkat NFκB luciferase reporter assay overexpressing OX40 (FIG. 5B). Full-length anti-OX40 huIgG1 mAb (valency of 2) was used as the negative control. As expected, no activity was seen for either the anti-OX40 huIgG or the NLP alone over the entire concentration range tested (FIG. 5B—triangles and crosses, respectively). For the Fab-NLP conjugates, a low level of agonist activity was observed at a Fab valency of 3.8 (FIG. 5B—stars). In contrast, highly potent activity was observed when the valency was increased to 8.3 Fabs/NLP (FIG. 5B—squares) and only a marginal increase in activity was observed at valencies higher than that. These results suggest that the minimum valency for strong potency is around 8 and are consistent with the aforementioned study comparing the agonist activity of molecules with valencies of 4 and 12 (45). It is worth noting that the data in FIG. 5B was displayed relative to the Fab concentration and not the NLP concentration. When we analyze the data based on NLP concentration, the curve (FIG. 5C) and corresponding EC50 (FIG. 5D) of the Fab/NLP complex decreased by a factor of ˜4 as the Fab valency was increased from 3.8 to 29.4. These findings suggest that the overall potency of the Fab-NLP conjugate increased with increasing valency. In conclusion, these findings demonstrate successful use of this platform to potently activate pathways requiring high valencies, which may not be achieved using engineered mAb platforms.

### Example 5: Serum Stability of NLP-Fab Conjugates

Labeling NLP and Fab-NLP with AF488

NLPs and Fab-NLP conjugates were generated as described above. Prior to SEC purification of the naked NLP or Fab-NLP conjugate, samples were labeled with AF488 via free lysines by incubation with NHS activated AF488 for 2-4 hours at a NHS-AF488: total protein ratio of 5. After this incubation period, unreacted NHS-AF488 was quenched with the addition of 2× molar excess of Tris relative to total NHS-AF488 in the reaction. The AF488 labeled NLPs were than purified from unreacted AF488 by SEC on an AKTA Avant system using an S200 10/300 Increase column.

### SEC Analysis of NLP and Fab-NLP Conjugates in 50% Serum

AF488 labeled NLP and Fab-NLP conjugates were incubated in PBS buffer containing 50% serum and analyzed by SEC (Acclaim SEC 1000, Thermo Fisher Scientific) at various time points post incubation in PBS buffer. The absorbance of the labeled NLP samples was monitored at a wavelength of 495 nm to avoid interfering absorbance from serum proteins and constituents. The peak observed between 6-9 minutes was attributed to the NLP and NLP-Fab conjugate and the peak between 10-13 minutes was attributed to free unconjugated Fab or apoE422k that had disassociated from the NLP. The area under the NLP peak at the various time points tested was normalized to the peak area at time 0 hr and these normalized values were used to determine the kinetics of NLP degradation.

### Results and Discussion for Example 5

### Fab-NLP Serum Stability

The NLP platform here can be used an in vivo delivery vehicle, despite reports of other NLP structures having low stability in complex biological matrices (20, 67, 68). In one previous study, the half-life of DOPC NLPs in 50% serum at 37° C. was reported to be between 3-6 hours (20), which is significantly lower than the systemic half-life of nanoparticles (24-48 hours). To address this limitation, a previous study evaluated the effect of a crosslinkable lipid component on overall NLP stability and demonstrated that crosslinking the bilayer core significantly improved stability (68). More specifically, no NLP degradation was observed over a 48-hour time period in 100% serum, whereas very rapid degradation (˜10 min) was observed in the absence of crosslinking (68). Although promising, one of the drawbacks of this method is an increased safety risk due to the addition of the non-natural crosslinked lipid, which has no known natural degradation or biotransformation pathway.

Given the importance of NLP stability for its application as an in vivo delivery vehicle, NLP-Fab conjugate stability was assessed in 50% serum, which was selected to mimic in vivo conditions, using the Rabbit Fab surrogate molecule. To evaluate NLP stability and integrity, Fab-NLP conjugates were labeled with AF488 and analyzed by SEC at an absorbance of 495 nm, which was chosen for its non-overlapping spectrophotometric signature with the intrinsic absorbance of serum proteins and constituents in the serum/NLP-Fab sample. Example SEC traces of the NLP alone are shown in FIG. 6A. The retention time of the intact NLP (˜8 min) was distinct from free apoE422k released from dissociated NLPs (8 min vs. 11.75 min, respectively), and thus the stability and integrity of the NLP could be readily followed over time by SEC. It is worth noting that the minor shoulder peak observed at a retention time of ˜6.25 min was due to aggregate in the serum and was background subtracted for analysis.

To evaluate the effect of Fab loading on stability, Fab-NLP conjugates with varying amounts of Fab (0, 2, 7, 16 and 32 Fab/NLP) were labeled with AF488, incubated in 50% serum at 37° C. and analyzed by SEC at different time points ranging from 0-24 h. Fab-NLP SEC peak areas were normalized relative to peak area at time 0 h to allow for comparison across the different Fab-NLP conjugates. Consistent with previously published studies (20, 67), NLP alone (FIG. 6B triangles) initially degraded rapidly with only 50% of the material remaining after 3 hours, followed by slower degradation over the remaining 24 hour incubation period. A very similar trend was also observed for the Fab-NLP conjugate at a Fab:NLP loading density of 2 (FIG. 6B circles). However, a significant improvement in NLP stability was observed when the Fab:NLP loading density was increased to 7 (FIG. 6B, squares), where the initial signature rapid decrease in stability was not observed and instead a slow gradual decrease dominated the entire 24 hours with 63% of the Fab-NLP remaining intact after incubation at 37° C. in 50% serum. A similar trend was also observed at Fab:NLP loading densities of 16 and 32 (FIG. 6B stars and diamonds respectively), with a further increase in stability detected, where greater than 70% of the Fab-NLP conjugates remained intact at the end of the 24 hour incubation period.

The improved stability of the Fab-NLP conjugate suggests that access to the NLP surface is linked to degradation and the conjugated Fabs may be shielding the NLP from serum proteins. Interactions with serum proteins such as albumin, as well as natural HDL and LDL particles, with the hydrophobic lipid bilayer core of the NLP are believed to be a main driver of degradation of NLPs in vivo or under in vitro serum conditions. These interactions are believed to disrupt the lipid-lipid and lipid-apolipoprotein hydrophobic van der Waals interactions that keep the NLP assembled and intact. This hypothesis is further supported by previous studies where crosslinking either the lipid bilayer core (68) or the apolipoprotein (69) was shown to significantly improved NLP stability. Despite these findings, less is known about the impact of serum protease activity on NLP stability. It is possible that the apolipoprotein is cleaved by serum proteins causing nanoparticle disassembly. However, given that Fab conjugation to the NLP surface as well as crosslinking of the lipid bilayer core and apolipoprotein were shown to improve stability, it seems likely that the main driver of NLP disassembly is disruption of van der Waals interactions as opposed to serum protease cleavage of the apolipoprotein. Surprisingly, a significant improvement in stability was observed at a relatively low Fab density of 7, which corresponds to ˜25% surface area coverage (assuming a Fab surface area of ˜2.2 nm²). These data suggest that a minimal number of surface-protecting proteins are sufficient to have a significant impact on NLP stability. Importantly, this is achieved without use of non-natural crosslinkers.

One of the difficulties expected in using NLPs as an in vivo drug delivery vehicle was the overall stability of the particle in a complex biological matrix such as serum. However, based on the results described above, this issue surprisingly appears to be mitigated when assembled as a Fab-NLP conjugate at a Fab loading density higher than a threshold value of ˜7 Fabs:NLP. Therefore modifications, such as lipid crosslinking which has the potential to introduce additional toxicity liabilities, are not needed to achieve stability during delivery of Fab-NLP conjugates.

### Summary:

In this study, we describe the development, optimization, and characterization of Fab-NLP conjugates. NLPs were generated with maleimide reactive lipids for conjugation to a Fab with a C-terminal cysteine. Maleimide reactive lipids were shown to conjugate to the apolipoprotein, most likely through lysine residues, when assembled at pH 7.4. However, this undesirable reaction was not observed when the NLPs were assembled at pH 6. Site-specific Fab conjugation conditions were optimized and conjugation of up to 30 Fab per NLP was demonstrated. Interestingly, conjugation of higher numbers of Fabs had minimal impact on NLP hydrodynamic radius (i.e., NLP diameter) and a significant impact on NLP molecular weight, indicating that particle size is largely dictated by the discoidal shape of the NLP.

Fab-NLP viscosity and its stability upon lyophilization were also evaluated as an assessment of the manufacturability of the Fab-NLP. That is, compatibility of the Fab-NLP platform with established manufacturing processes was evaluated in two ways: by comparing its visco-elastic behavior with another multimerization technology (Fab-PEG octamer conjugate) across a range of Fab concentrations relevant during manufacturing and by assessing its stability upon lyophilization. Significantly higher Fab concentrations were achieved for Fab-NLP conjugates relative to another multivalent format (Fab-PEG conjugates) and lyophilization did not reveal any stability nor activity liabilities. Moreover, Fab conjugation to the NLP was not found to have an impact on Fab activity, in either an inhibitory and agonist setting, and we were able to leverage the Fab loading capacity to activate an agonist pathway requiring high valency for potency. Finally, the stability of the Fab-NLP conjugates was evaluated in 50% serum and Fab-NLPs demonstrated increased stability, with >63% of Fab-NLP remaining intact after 24 hours at Fab per particle ratios of 7 or greater. Our findings establish Fab-NLPs as a platform for the targeted delivery of Fabs in a multivalent format and that Fab-NLPs are compatible with established manufacturing processes.

The combined results of this study demonstrate that the NLP is a well-behaved, versatile platform, very well suited for multimerization of a Fab or Fab-like molecule. These results evidence that NLPs are a surprisingly useful platform for the targeted delivery of Fabs in a multivalent format and is compatible with established manufacturing processes.

### REFERENCES FOR EXAMPLES 1-5

(1) Cho, K., Wang, X., Nie, S., Chen, Z. G., and Shin, D. M. (2008) Therapeutic nanoparticles for drug delivery in cancer. Clin Cancer Res 14, 1310-6.
(2) Mareeva, T., Wanjalla, C., Schnell, M. J., and Sykulev, Y. (2010) A novel composite immunotoxin that suppresses rabies virus production by the infected cells. J Immunol Methods 353, 78-86.
(3) Bhatt, R., de Vries, P., Tulinsky, J., Bellamy, G., Baker, B., Singer, J. W., and Klein, P. (2003) Synthesis and in vivo antitumor activity of poly(1-glutamic acid) conjugates of 20S-camptothecin. J Med Chem 46, 190-3.
(4) Kim, T. Y., Kim, D. W., Chung, J. Y., Shin, S. G., Kim, S. C., Heo, D. S., Kim, N. K., and Bang, Y. J. (2004) Phase I and pharmacokinetic study of Genexol-PM, a cremophor-free, polymeric micelle-formulated paclitaxel, in patients with advanced malignancies. Clin Cancer Res 10, 3708-16.
(5) Malik, N., Evagorou, E. G., and Duncan, R. (1999) Dendrimer-platinate: a novel approach to cancer chemotherapy. Anticancer Drugs 10, 767-76.
(6) Markman, M. (2006) Pegylated liposomal doxorubicin in the treatment of cancers of the breast and ovary. Expert Opin Pharmacother 7, 1469-74.
(7) Manchester, M., and Singh, P. (2006) Virus-based nanoparticles (VNPs): platform technologies for diagnostic imaging. Adv Drug Deliv Rev 58, 1505-22.
(8) Wu, W., Wieckowski, S., Pastorin, G., Benincasa, M., Klumpp, C., Briand, J. P.,
Gennaro, R., Prato, M., and Bianco, A. (2005) Targeted delivery of amphotericin B to cells by using functionalized carbon nanotubes. Angew Chem Int Ed Engl 44, 6358-62.
(9) Tufteland, M., Ren, G., and Ryan, R. O. (2008) Nanodisks derived from amphotericin B lipid complex. J Pharm Sci 97, 4425-32.
(10) Yuan, Y., Wang, W., Wang, B., Zhu, H., Zhang, B., and Feng, M. (2013) Delivery of hydrophilic drug doxorubicin hydrochloride-targeted liver using apoAI as carrier. J Drug Target 21, 367-74.
(11) Ding, Y., Wang, W., Feng, M., Wang, Y., Zhou, J., Ding, X., Zhou, X., Liu, C., Wang, R., and Zhang, Q. (2012) A biomimetic nanovector-mediated targeted cholesterol-conjugated siRNA delivery for tumor gene therapy. Biomaterials 33, 8893-905.
(12) Gaidukov, L., Bar, D., Yacobson, S., Naftali, E., Kaufman, O., Tabakman, R., Tawfik, D. S., and Levy-Nissenbaum, E. (2009) In vivo administration of BL-3050: highly stable engineered PON1-HDL complexes. BMC Clin Pharmacol 9, 18.
(13) Frias, J. C., Ma, Y., Williams, K. J., Fayad, Z. A., and Fisher, E. A. (2006) Properties of a versatile nanoparticle platform contrast agent to image and characterize atherosclerotic plaques by magnetic resonance imaging. Nano Lett 6, 2220-4.
(14) Fischer, N. O., Rasley, A., Corzett, M., Hwang, M. H., Hoeprich, P. D., and Blanchette, C. D. (2013) Colocalized delivery of adjuvant and antigen using nanolipoprotein particles enhances the immune response to recombinant antigens. J Am Chem Soc 135, 2044-7.
(15) Bhattacharya, P., Grimme, S., Ganesh, B., Gopisetty, A., Sheng, J. R., Martinez, O., Jayarama, S., Artinger, M., Meriggioli, M., and Prabhakar, B. S. (2010) Nanodisc-incorporated hemagglutinin provides protective immunity against influenza virus infection. J Virol 84, 361-71.
(16) Fischer, N. O., Infante, E., Ishikawa, T., Blanchette, C. D., Bourne, N., Hoeprich, P. D., and Mason, P. W. (2010) Conjugation to nickel-chelating nanolipoprotein particles increases the potency and efficacy of subunit vaccines to prevent West Nile encephalitis. Bioconjug Chem 21, 1018-22.
(17) Weilhammer, D. R., Blanchette, C. D., Fischer, N. O., Alam, S., Loots, G. G., Corzett, M., Thomas, C., Lychak, C., Dunkle, A. D., Ruitenberg, J. J., Ghanekar, S. A., Sant, A. J., and Rasley, A. (2013) The use of nanolipoprotein particles to enhance the immunostimulatory properties of innate immune agonists against lethal influenza challenge. Biomaterials 34, 10305-18.
(18) Fischer, N. O., Blanchette, C. D., Segelke, B. W., Corzett, M., Chromy, B. A., Kuhn, E. A., Bench, G., and Hoeprich, P. D. (2010) Isolation, characterization, and stability of discretely-sized nanolipoprotein particles assembled with apolipophorin-III. PLoS One 5, e11643.
(19) Blanchette, C. D., Law, R., Benner, W. H., Pesavento, J. B., Cappuccio, J. A., Walsworth, V., Kuhn, E. A., Corzett, M., Chromy, B. A., Segelke, B. W., Coleman, M. A., Bench, G., Hoeprich, P. D., and Sulchek, T. A. (2008) Quantifying size distributions of nanolipoprotein particles with single-particle analysis and molecular dynamic simulations. J Lipid Res 49, 1420-30.
(20) Fischer, N. O., Weilhammer, D. R., Dunkle, A., Thomas, C., Hwang, M., Corzett, M., Lychak, C., Mayer, W., Urbin, S., Collette, N., Chiun Chang, J., Loots, G. G., Rasley, A., and Blanchette, C. D. (2014) Evaluation of nanolipoprotein particles (NLPs) as an in vivo delivery platform. PLoS One 9, c93342.
(21) Cappuccio, J. A., Blanchette, C. D., Sulchek, T. A., Arroyo, E. S., Kralj, J. M., Hinz, A. K., Kuhn, E. A., Chromy, B. A., Segelke, B. W., Rothschild, K. J., Fletcher, J. E., Katzen, F., Peterson, T. C., Kudlicki, W. A., Bench, G., Hoeprich, P. D., and Coleman, M. A. (2008) Cell-free co-expression of functional membrane proteins and apolipoprotein, forming soluble nanolipoprotein particles. Mol Cell Proteomics 7, 2246-53.
(22) Cappuccio, J. A., Hinz, A. K., Kuhn, E. A., Fletcher, J. E., Arroyo, E. S., Henderson, P. T., Blanchette, C. D., Walsworth, V. L., Corzett, M. H., Law, R. J., Pesavento, J. B., Segelke, B. W., Sulchek, T. A., Chromy, B. A., Katzen, F., Peterson, T., Bench, G., Kudlicki, W., Hoeprich, P. D., Jr., and Coleman, M. A. (2009) Cell-free expression for nanolipoprotein particles: building a high-throughput membrane protein solubility platform. Methods Mol Biol 498, 273-96.
(23) Wadsater, M., Laursen, T., Singha, A., Hatzakis, N. S., Stamou, D., Barker, R., Mortensen. K., Feidenhans'l, R., Moller, B. L., and Cardenas, M. (2012) Monitoring shifts in the conformation equilibrium of the membrane protein cytochrome P450 reductase (POR) in nanodiscs. J Biol Chem 287, 34596-603.
(24) Justesen, B. H., Laursen, T., Weber, G., Fuglsang, A. T., Moller, B. L., and Pomorski, T. G. (2013) Isolation of monodisperse nanodisc-reconstituted membrane proteins using free flow electrophoresis. Anal Chem 85, 3497-500.
(25) Baylon, J. L., Lenov, I. L., Sligar, S. G., and Tajkhorshid, E. (2013) Characterizing the membrane-bound state of cytochrome P450 3A4: structure, depth of insertion, and orientation. J Am Chem Soc 135, 8542-51.
(26) Gao, T., Petrlova, J., He, W., Huser, T., Kudlick, W., Voss, J., and Coleman, M. A. (2012) Characterization of de novo synthesized GPCRs supported in nanolipoprotein discs. PLoS One 7, e44911.
(27) Akkaladevi, N., Hinton-Chollet, L., Katayama, H., Mitchell, J., Szerszen, L.,
Mukherjee, S., Gogol, E. P., Pentelute, B. L., Collier, R. J., and Fisher, M. T. (2013) Assembly of anthrax toxin pore: lethal-factor complexes into lipid nanodiscs. Protein Sci 22, 492-501.
(28) Tufteland, M., Pesavento, J. B., Bermingham, R. L., Hoeprich, P. D., Jr., and Ryan, R. O. (2007) Peptide stabilized amphotericin B nanodisks. Peptides 28, 741-6.
(29) Jia, J., Xiao, Y., Liu, J., Zhang, W., He, H., Chen, L., and Zhang, M. (2012) Preparation, characterizations, and in vitro metabolic processes of paclitaxel-loaded discoidal recombinant high-density lipoproteins. J Pharm Sci 101, 2900-8.
(30) Weilhammer, D., Dunkle, A. D., Blanchette, C. D., Fischer, N. O., Corzett, M., Lehmann, D., Boone, T., Hoeprich, P., Driks, A., and Rasley, A. (2017) Enhancement of antigen-specific CD4(+) and CD8(+) T cell responses using a self-assembled biologic nanolipoprotein particle vaccine. Vaccine 35, 1475-1481.
(31) Blanchette, C. D., Fischer, N. O., Corzett, M., Bench, G., and Hoeprich, P. D. (2010)
Kinetic analysis of his-tagged protein binding to nickel-chelating nanolipoprotein particles. Bioconjug Chem 21, 1321-30.
(32) Kuai, R., Sun, X., Yuan, W., Xu, Y., Schwendeman, A., and Moon, J. J. (2018) Subcutaneous Nanodisc Vaccination with Neoantigens for Combination Cancer Immunotherapy. Bioconjug Chem 29, 771-775.
(33) Merino, M., Zalba, S., and Garrido, M. J. (2018) Immunoliposomes in clinical oncology: State of the art and future perspectives. J Control Release 275, 162-176.
(34) Eloy, J. O., Petrilli, R., Trevizan, L. N. F., and Chorilli, M. (2017) Immunoliposomes: A review on functionalization strategies and targets for drug delivery. Colloids Surf B Biointerfaces 159, 454-467.
(35) Noble, G. T., Stefanick, J. F., Ashley, J. D., Kiziltepe, T., and Bilgicer, B. (2014) Ligand-targeted liposome design: challenges and fundamental considerations. Trends Biotechnol 32, 32-45.
(36) Alibakhshi, A., Abarghooi Kahaki, F., Ahangarzadeh, S., Yaghoobi, H., Yarian, F., Arezumand, R., Ranjbari, J., Mokhtarzadeh, A., and de la Guardia, M. (2017) Targeted cancer therapy through antibody fragments-decorated nanomedicines. J Control Release 268, 323-334.
(37) Ohradanova-Repic, A., Nogueira, E., Hartl, I., Gomes, A. C., Preto, A., Steinhuber, E., Muhlgrabner, V., Repic, M., Kuttke, M., Zwirzitz, A., Prouza, M., Suchanek, M.,
Wozniak-Knopp, G., Horejsi, V., Schabbauer, G., Cavaco-Paulo, A., and Stockinger, H. (2018) Fab antibody fragment-functionalized liposomes for specific targeting of antigen-positive cells. Nanomedicine 14, 123-130.
(38) Hoang, B., Ekdawi, S. N., Reilly, R. M., and Allen, C. (2013) Active targeting of block copolymer micelles with trastuzumab Fab fragments and nuclear localization signal leads to increased tumor uptake and nuclear localization in HER2-overexpressing xenografts. Mol Pharm 10, 4229-41.
(39) Mamot, C., Drummond, D. C., Noble, C. O., Kallab, V., Guo, Z., Hong, K., Kirpotin, D. B., and Park, J. W. (2005) Epidermal growth factor receptor-targeted immunoliposomes significantly enhance the efficacy of multiple anticancer drugs in vivo. Cancer Res 65, 11631-8.
(40) Hoshyar, N., Gray, S., Han, H., and Bao, G. (2016) The effect of nanoparticle size on in vivo pharmacokinetics and cellular interaction. Nanomedicine (Lond) 11, 673-92.
(41) Steeland, S., Vandenbroucke, R. E., and Libert, C. (2016) Nanobodies as therapeutics: big opportunities for small antibodies. Drug Discov Today 21, 1076-113.
(42) Redmond, W. L., and Weinberg, A. D. (2007) Targeting OX40 and OX40L for the treatment of autoimmunity and cancer. Crit Rev Immunol 27, 415-36.
(43) Cuesta, A. M., Sainz-Pastor, N., Bonet, J., Oliva, B., and Alvarez-Vallina, L. (2010) Multivalent antibodies: when design surpasses evolution. Trends Biotechnol 28, 355-62.
(44) Nunez-Prado, N., Compte, M., Harwood, S., Alvarez-Mendez, A., Lykkemark, S., Sanz, L., and Alvarez-Vallina, L. (2015) The coming of age of engineered multivalent antibodies. Drug Discov Today 20, 588-94.
(45) Yang, Y., Yeh, S. H., Madireddi, S., Matochko, W. L., Gu, C., Pacheco Sanchez, P., Ultsch, M., De Leon Boenig, G., Harris, S. F., Leonard, B., Scales, S. J., Zhu, J. W., Christensen, E., Hang, J. Q., Brezski, R. J., Marsters, S., Ashkenazi, A., Sukumaran, S., Chiu, H., Cubas, R., Kim, J. M., and Lazar, G. A. (2019) Tetravalent biepitopic targeting enables intrinsic antibody agonism of tumor necrosis factor receptor superfamily members. MAbs 11, 996-1011.
(46) Fischer, N. O., Blanchette, C. D., Chromy, B. A., Kuhn, E. A., Segelke, B. W., Corzett, M., Bench, G., Mason, P. W., and Hoeprich, P. D. (2009) Immobilization of His-tagged proteins on nickel-chelating nanolipoprotein particles. Bioconjug Chem 20, 460-5.
(47) Shatz, W., Hass, P. E., Peer, N., Paluch, M. T., Blanchette, C., Han, G., Sandoval, W., Morando, A., Loyet, K. M., Bantseev, V., Booler, H., Crowell, S., Kamath, A., Scheer, J. M., and Kelley, R. F. (2019) Identification and characterization of an octameric PEG-protein conjugate system for intravitreal long-acting delivery to the back of the eye. PLoS One 14, e0218613.
(48) Shatz, W., Hass, P. E., Mathieu, M., Kim, H. S., Leach, K., Zhou, M., Crawford, Y., Shen, A., Wang, K., Chang, D. P., Maia, M., Crowell, S. R., Dickmann, L., Scheer, J. M., and Kelley, R. F. (2016) Contribution of Antibody Hydrodynamic Size to Vitreal Clearance Revealed through Rabbit Studies Using a Species-Matched Fab. Mol Pharm 13, 2996-3003.
(49) Tesar, D., Luoma, J., Wyatt, E. A., Shi, C., Shatz, W., Hass, P. E., Mathieu, M., Yi, L., Corn, J. E., Maass, K. F., Wang, K., Dion, M. Z., Andersen, N., Loyet, K. M., van Lookeren Campagne, M., Rajagopal, K., Dickmann, L., Scheer, J. M., and Kelley, R. F. (2017) Protein engineering to increase the potential of a therapeutic antibody Fab for long-acting delivery to the eye. MAbs 9, 1297-1305.
(50) Hermanson, G. T. (2008) Bioconjugate Techniques, pp 283.
(51) Partis, M. D., Griffiths, D. G., Roberts, G. C., and Beechey, R. B. (1983) Cross-Linking of Protein by Omega-Maleimido Alkanoyl N-Hydroxysuccinimido Esters. J Protein Chem 2, 263-277.
(52) Hermanson, G. T. (2008) Bioconjugate Techniques, pp 767.
(53) Dong, J., Peters-Libeu, C. A., Weisgraber, K. H., Segelke, B. W., Rupp, B., Capila, I., Hernaiz, M. J., LeBrun, L. A., and Linhardt, R. J. (2001) Interaction of the N-terminal domain of apolipoprotein E4 with heparin. Biochemistry 40, 2826-34.
(54) Isom, D. G., Castaneda, C. A., Cannon, B. R., and Garcia-Moreno, B. (2011) Large shifts in pKa values of lysine residues buried inside a protein. Proc Natl Acad Sci USA 108, 5260-5.
(55) Panahi, A., and Brooks, C. L., 3rd. (2015) Membrane environment modulates the pKa values of transmembrane helices. J Phys Chem B 119, 4601-7.
(56) Strong, R. K., Campbell, R., Rose, D. R., Petsko, G. A., Sharon, J., and Margolies, M. N. (1991) Three-dimensional structure of murine anti-p-azophenylarsonate Fab 36-71. 1. X-ray crystallography, site-directed mutagenesis, and modeling of the complex with hapten. Biochemistry 30, 3739-48.
(57) Gao, T., Blanchette, C. D., He, W., Bourguet, F., Ly, S., Katzen, F., Kudlicki, W. A., Henderson, P. T., Laurence, T. A., Huser, T., and Coleman, M. A. (2011) Characterizing diffusion dynamics of a membrane protein associated with nanolipoproteins using fluorescence correlation spectroscopy. Protein Sci 20, 437-47.
(58) Henein, C., Awwad, S., Ibeanu, N., Vlatakis, S., Brocchini, S., Tee Khaw, P., and Bouremel, Y. (2019) Hydrodynamics of Intravitreal Injections into Liquid Vitreous Substitutes. Pharmaceutics 11.
(59) Dias, C., Abosaleem, B., Crispino, C., Gao, B., and Shaywitz, A. (2015) Tolerability of High-Volume Subcutaneous Injections of a Viscous Placebo Buffer: A Randomized, Crossover Study in Healthy Subjects. AAPS PharmSciTech 16, 1101-7.
(60) Mathaes, R., Koulov, A., Joerg, S., and Mahler, H. C. (2016) Subcutaneous Injection Volume of Biopharmaceuticals-Pushing the Boundaries. J Pharm Sci 105, 2255-9.
(61) Zarzar, J., Shatz, W., Peer, N., Taing, R., McGarry, B., Liu, Y., Greene, D. G., and Zarraga, I. E. (2018) Impact of polymer geometry on the interactions of protein-PEG conjugates. Biophys Chem 236, 22-30.
(62) Chen, C., Han, D., Cai, C., and Tang. X. (2010) An overview of liposome lyophilization and its future potential. J Control Release 142, 299-311.
(63) Fernandez-Moure, J., Maisha, N., Lavik, E. B., and Cannon, J. W. (2018) The Chemistry of Lyophilized Blood Products. Bioconjug Chem 29, 2150-2160.
(64) Franze, S., Selmin, F., Samaritani, E., Minghetti, P., and Cilurzo, F. (2018) Lyophilization of Liposomal Formulations: Still Necessary, Still Challenging. Pharmaceutics 10.
(65) Rusmini, F., Zhong, Z., and Feijen, J. (2007) Protein immobilization strategies for protein biochips. Biomacromolecules 8, 1775-89.
(66) Abad, J. M., Mertens, S. F., Pita, M., Fernandez, V. M., and Schiffrin, D. J. (2005) Functionalization of thioctic acid-capped gold nanoparticles for specific immobilization of histidine-tagged proteins. J Am Chem Soc 127, 5689-94.
(67) Gilmore, S. F., Carpenter, T. S., Ingolfsson, H. I., Peters, S. K. G., Henderson, P. T., Blanchette. C. D., and Fischer, N. O. (2018) Lipid composition dictates serum stability of reconstituted high-density lipoproteins: implications for in vivo applications. Nanoscale 10, 7420-7430.
(68) Gilmore, S. F., Blanchette, C. D., Scharadin, T. M., Hura, G. L., Rasley, A., Corzett, M., Pan, C. X., Fischer, N. O., and Henderson, P. T. (2016) Lipid Cross-Linking of Nanolipoprotein Particles Substantially Enhances Serum Stability and Cellular Uptake. ACS Appl Mater Interfaces 8, 20549-57.
(69) Nasr, M. L., and Wagner, G. (2018) Covalently circularized nanodiscs; challenges and applications. Curr Opin Struct Biol 51, 129-134.

### Example 6: Materials and Methods of Examples 7-11

### Materials

Reagents used in Examples 7-11 were obtained as discussed in Example 1.

Protein Expression and Purification of apoE422k and Fabs

ApoE422k was expressed in E. coli cells under shake flask conditions as described previously (see references 18 and 19 below) using established expression plasmids and methods. Briefly, apoE422k was expressed with a 6×His tag and purified over a Ni-NTA column (XK16/20 3 ml) followed by size exclusion chromatography (SEC) (Superdex 75 16/60). The column was washed and protein bound in 50 mM phosphate buffer, 200 mM NaCl, 10 mM imidazole, pH 8 (Buffer A). The column bound protein was washed extensively (20 column volumes) with Buffer A +0.2% Triton X114+0.2% Triton X100 and eluted with 50 mM phosphate buffer, 200 mM NaCl, 400 mM imidazole, pH 8. Fractions collected from the eluted peak were filtered and concentrated with a 3 kDa molecular weight cutoff spin concentrators. The His tag was removed through Tobacco Etch Virus nuclear-inclusion-a endopeptidase (TEV protease) digestion (TEV tag was added on N-terminus between the His tag and protein sequence). The TEV protease was added at a apoE422k:TEV weight ratio of 100 to 1. The cleaved protein was purified from the TEV protease, which contained a His tag, by passing the reaction mixture over a Ni-NTA column (XK 16/20 3 ml). The pooled protein was concentrated and run on SEC (Superdex 75 16/60) in PBS. SEC fractions were collected and analyzed for protein identity and aggregation by mass spectrometry and SEC, respectively. Fractions with the correct molecular weight (MW) and aggregation (<5%) were pooled and protein concentration was determined by absorbance at 280.

Fab constructs were designed with a C-terminal cysteine (referred to as “Fab” for simplicity) to enable site-specific conjugation to the CKP-NLP. Fab conjugation to CKP-NLPs was evaluated using human anti-factor D (AFD) Fab as a surrogate, which were purified as described previously (see reference 18 below). All purified Fabs were de-cysteinylated using 20 mM DTT to reduce cysteine conjugates and re-oxidized with 6.5 mM glutathione (GSH).

Samples were then buffer exchanged and washed and stored in 200 mM arginine succinate pH to limit further cysteinylation.

### Synthesis and Characterization of C16-EETI-II.

Linear EETI-II or EETI-II-C16 peptides were chemically synthesized using standard 9-fluorenylmethoxycarbonyl protocols on solid phase as described in reference 20 below. Optimal folding conditions were identified using small-scale folding assays and LC-MS analysis. The obtained crude linear peptides were oxidized in an optimal folding buffer (0.1 M ammonium bicarbonate, pH 9.0, 2 mM reduced glutathione, 0.5 mM oxidized glutathione, 4% DMSO for EETI-II; 0.1 M ammonium bicarbonate, pH 9.0, 1 mM reduced glutathione, 50% DMSO for C16-EETI-II) at 0.5 mg/ml for 24 hours at RT with shaking. C18 Sep-Pak (Waters, cat #WAT043345) was used to remove excess salt and the lyophilized product was purified by RP-HPLC (Agilent Technologies, Santa Clara, CA) using a C18 column for EETI-11 (referred to as “CKP” for simplicity) and a C4 column for EETI-TT-C16 (referred to as “CKP-C16” for simplicity). The identity of the purified peptides was confirmed using a LC-MS system (Agilent Technologies, Santa Clara, CA) and their purities confirmed to be >95%.

### CKP-NLP Assembly and Purification

To permit CKP incorporation into the NLP, the CKP was appended with a C16 fatty acid tag (CKP-C16) as described above. As described previously, NLP incorporation with molecules synthesized with a hydrophobic tag is based on partitioning of the hydrophobic tag within the core of the lipid bilayer effectively anchoring the molecule to the NLP surface (see references 9, 10 below). CKP-NLP were assembled as described previously with slight modifications (see references 9, 10, 18 below). Briefly, the total lipid to apoE422k molar ratio during assembly was 80:1, which was previously shown to result in a relatively homogeneous NLP population (see references 6, 11, 19, 21 below). CKP-NLPs were assembled with the indicated molar ratios of DOPC and CKP-C16 (no Fab conjugation) or DOPC, DSPE-PEG-Mal (Fab conjugation) and CKP-C16 as described in the Examples below. These lipids were either prepared or obtained in chloroform or methanol and stored as aliquots in eppendorf tubes. Chloroform was removed under a stream of N₂ with agitation to form a thin lipid film. Lipids were solubilized in 50 mM sodium phosphate buffer, pH 6.0, 150 mM NaCl using 80 mM sodium cholate. After addition of apoE422k (150 μM in final assembly volume), samples were incubated at room temperature for at least 1 hour. Cholate was then removed by incubation with biobeads (Sigma-Aldrich) for 2 hours with rocking in 500 μL costar 0.22 μM spin filters. After two hours of rocking, the samples were centrifuged at 200 g for 5 minutes and the filtrate containing the NLPs was collected. CKP-NLPs that were not going to be used for Fab conjugation were purified over SEC using an AKTA Avant system and S200 10/300 Increase column. CKP-NLPs that were going to be used to generate Fab-CKP-NLP conjugates were first conjugated to Fab as described below before SEC purification.

### Fab-CKP-NLP Conjugation and Purification

Fabs were conjugated to CKP-NLPs as described previously with slight modifications (see reference 18 below). Briefly, after the cholate removal step, apoE422k concentration for the CKP-NLPs was determined by HPLC as described below. Thiol containing Fabs were conjugated to maleimide functionalized CKP-NLPs in 50 mM sodium phosphate buffer, pH 6.0, 150 mM NaCl at Fab:NLP molar ratios ranging from 0-160, which was calculated based on the apoE422k concentration and assumption of 4 apoE422k/NLP (see reference 6 below). This conjugation buffer was selected to limit intra-NLP crosslinking between the maleimide and apoE422k as described previously (see reference 18 below). Conjugation reactions were always performed the same day the CKP-NLPs were assembled to limit hydrolysis of the maleimide. After the 2-4 hour reaction incubation period, n-acetylcysteine (NAC) was added at a 2-fold molar excess over DSPE-PEG-MCC to quench any unreacted maleimide. The resulting Fab-CKP-NLP conjugates were purified using an AKTA Avant system and S200 10/300 Increase column. Each fraction across the Fab-CKP-NLP peak was analyzed by SEC-MALS as described below. Fractions were pooled based on MW and hydrodynamic radius (Rₕ) analysis to yield the most homogeneous Fab-CKP-NLP sample.

Human anti-factor D (AFD) Fab engineered with a C-terminal cysteine (referred to as “Fab” for simplicity) to enable site specific conjugation to the CKP-NLP was used as a model Fab. The Fab was expressed and purified as described previously (references 18, 22 below). The purified Fabs were reduced with 20 mM DTT to remove cysteine or GSH conjugates that form during expression and purification and then re-oxidized using 6.5 mM glutathione (GSH) to ensure the cysteine is free for conjugation to the maleimide functionalized CKP-NLPs. Samples were buffer exchanged, washed and stored in 200 mM arginine succinate pH 5 to limit further cysteinylation.

### HPLC Analysis of CKP-NLP and Fab-CKP-NLP Conjugates

ApoE422k, CKP, and Fab concentration in the CKP-NLP and Fab-CKP-NLP conjugate was analyzed by HPLC using an Agilent 1290 Infinity Bio-inert HPLC. A Kinetex 2.6 μm XB-C16 column (Phenomenex), heated to 80° C., was used to analyze the injected NLP samples. The solvent was run as a gradient from a mixture of 30% methanol, 70% water and 0% 2-propanol to 100% 2-propanol. All solvents had 0.05% trifluoroacetic acid. Gradients were optimized to allow for separation of all components. For the Fab-CKP-NLP conjugates, reduced conditions were needed because the intact Fab could not be separated from the free apoE422k with these gradient and buffer conditions. ApoE422k, Fab, and CKP-C16 concentrations were determined based on standard curves generated by injecting 1-16 μg of each component and integrating the area under the curve. Standard curves were generated based on both the A280 UV signal and evaporative light scattering detector (ELSD) signal.

### LCMS Analysis of CKP-NLP and Fab-CKP-NLP Conjugates

LCMS analysis of the CKP-NLP and Fab-CKP-NLP conjugate was performed using an Agilent 6230 ESI-TOF LC/MS. A Kinetex 2.6 μm XB-C18 column (Phenomenex, Torrance, CA), heated to 80° C., was used to analyze the injected NLP samples. The solvent was run as a gradient from a mixture of 30% methanol and 70% water to 100% 2-propanol. All solvents had 0.05% trifluoroacetic acid.

### SEC-MALS/QELS Analysis of CKP-NLP and Fab-CKP-NLP Conjugates

MW and Rₕ were determined as described previously (reference 18 below). Briefly, samples were injected onto an Acclaim SEC-1000 analytical SEC column (Thermo Fisher Scientific, Waltham, MA), with isocratic gradient of phosphate buffered saline (PBS) (with an additional 150 mM NaCl), coupled to a multi-angle light scattering system (MALS) (Wyatt Instruments, Santa Barbara, CA). The diffusion coefficients (D) were measured using quasi elastic light scattering (QELS) where fluctuations in intensity of laser light scattered were captured using a single photon counting module detecting at a 99.0° angle. The Stokes-Einstein relationship was used to calculate Rₕ from D. The Stokes-Einstein relationship assumes spherical shape and NLPs are discoidal and not spherical by TEM and AFM analysis (see reference 6 below). The estimated Rh therefore corresponds to a sphere that has the same diffusion coefficient as NLP. However, NLP shape in solution has recently been reported to be highly dynamic where NLPs can adopt a variety of different conformations beyond a static discoidal shape that would be more reflective of a spherical shaped particle (references 18, 23 below). For these reasons, it is presumed that the Rh values obtained from this analysis are accurate.

### Trypsin Enzymatic Assay

Assays were performed as described earlier with slight modifications (see reference 2 below). Briefly, the peptide NLP conjugates were incubated at the indicated concentration with trypsin (2 nM) for 30 min at room temperature. The substrate L-Arginine-7-amido-4-methylcoumarin (L-Arg-AMC) (75 μM) was added to the mixture and the proteolytic activity was measured immediately. Samples were run in triplicate and repeated twice. The data were analyzed using KaleidaGraph software.

Labeling CKP-NLP and Fab-CKP-NLP Conjugates with AF488

CKP-NLPs and Fab-CKP-NLP conjugates were generated as described above and prior to SEC purification (after cholate removal) samples were labeled with AF488 via free lysines by incubation with NHS activated AF488 for 2-4 hours at a NHS-AF488: total protein ratio of 5. Unreacted NHS-AF488 was then quenched with the addition of 10× molar excess of Tris-HCl buffer (pH 8) relative to total NHS-AF488 in the reaction. The AF488 labeled NLPs were than purified from unreacted AF488 by SEC on an AKTA Avant system using an S200 10/300 Increase column as described above.

### SEC Analysis of CKP-NLP and Fab-CKP-NLP Conjugates in 50% Serum

Degradation of AF488 labeled CKP-NLPs and Fab-CKP-NLPs was analyzed as described previously (see references 9, 18, 24, and 25 below). AF488 labeled CKP-NLP and Fab-CKP-NLP conjugates were incubated in PBS buffer containing 50% serum (pH 7.4) and analyzed by SEC (Acclaim SEC 1000, Thermo Fisher Scientific, Waltham, MA). The absorbance of the labeled NLP samples was monitored at a wavelength of 495 nm to avoid interfering absorbance from serum proteins and constituents. The peak observed between 5-5.5 minutes was attributed to the CKP-NLP and Fab-CKP-NLP conjugate and the peak between 6-6.5 minutes was attributed to free unconjugated Fab or apoE422k that had disassociated from the NLP. The area under the NLP peak at the various time points tested was normalized to the peak area at time 0 hr and these normalized values were used to determine the kinetics of CKP-NLP and Fab-CKP-NLP degradation.

### Example 7: Assembly, Purification and Characterization of CKP-NLPs

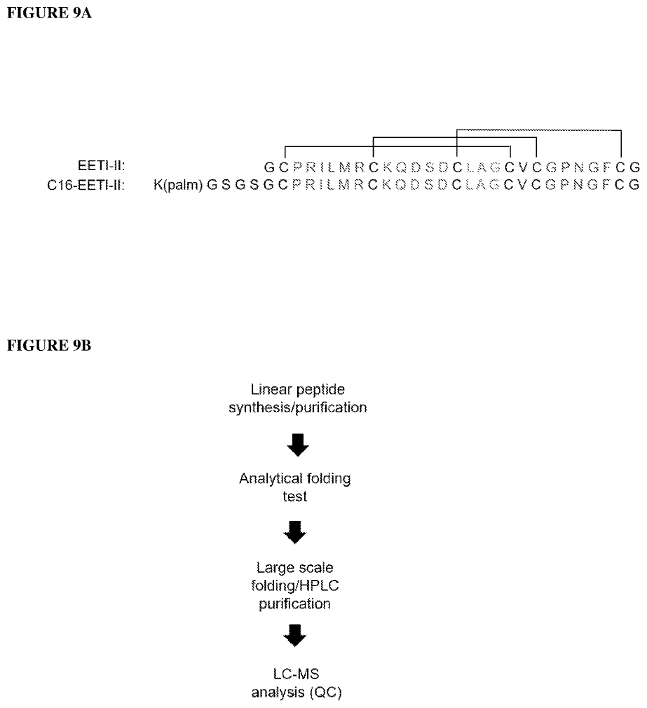

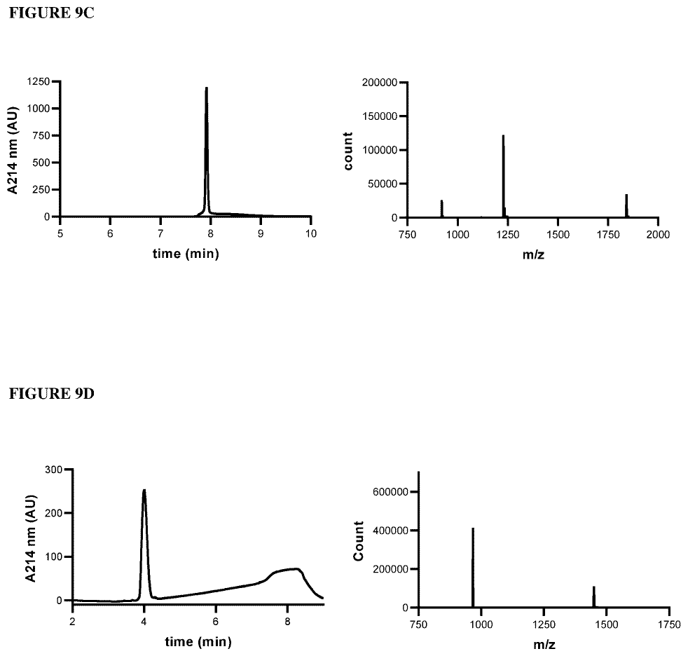

The cystine-knot peptide EETT-II (Ecballium elaterium trypsin inhibitor-II) is a potent trypsin inhibitor found in squirting cucumbers. To generate CKP-NLPs, a fatty acylated version of EETI-II was designed in which palmitic acid was conjugated to the epsilon amino group of a lysine side chain that was engineered at the N terminus of EETI-JI (FIG. 9A). First, linear C16-EETI-II was generated by solid-phase peptide synthesis. The fatty acid was incorporated as the last step during peptide synthesis before subsequent peptide cleavage from resin and deprotection. The obtained linear crude product was then screened in different buffers to identify the optimal folding conditions to generate a three disulfide-bonded product, as assessed by analytical LC-MS. This production strategy simplifies the purification process (instead of conjugating the fatty acid after CKP folding). See FIG. 9B. EETI-II-C16 was folded and purified to near homogeneity, producing EETI-II-C16 to multi milligram scale. The identity and purity of EETI-II-C16 was confirmed by LC-MS (FIG. 9C). The analytical RP-HPLC traces indicate that EETI-II-C16 is more hydrophobic than EETI-II, as reflected by the longer retention time compared to native EETI-JI (FIG. 9D). This is due to conjugation of the fatty acid moiety. -EETI-II-C16 still shows solubility in aqueous buffer (>0.5 mM in 60 mM phosphate buffer, pH 7.4). The purified EETI-II-16 demonstrated inhibitory potency against trypsin, consistent with predictions from structural studies that chemical modifications at the N terminus should be minimally disruptive to the activity of EETI-II. For simplicity, EETI-II and EETI-II-C16 are referred to herein as “CKP” and “CKP-C16,” respectively.

CKP-NLPs were generated through spontaneous self-assembly using DOPC as the helper lipid and cholate for lipid solubilization as illustrated in the schematic shown in FIG. 10A. Upon cholate removal, the self-assembled CKP-NLPs were purified by SEC. A typical SEC chromatogram of CKP-NLPs is shown in FIG. 10B. This CKP-NLP sample was assembled at a CKP-C16 molar ratio relative to DOPC of 10% (e.g. 90 mol % DOPC, 10 mol % CKP-C16), which corresponds to 40 CKPs/NLP assuming 4 apoE422k scaffolds/NLP (see reference 6 below). Fractions corresponding to the center cut of the SEC peak were collected, pooled and subjected to reversed-phase HPLC analysis. FIG. 10C shows an HPLC ELSD chromatogram of the CKP-NLP sample assembled with 10 mol % CKP-C16 after SEC purification. Three peaks were observed at retention times corresponding to 4.8, 5.3 and 8.5 min. Standard curves and LC/MS analysis were used to verify that the peaks observed at 4.8, 5.3 and 8.5 min corresponded to apoE422k (22.3 kDa), CKP-C16 (3.7 kDa) and DOPC (0.79 kDa), respectively. These findings show that the strategy described above results in the incorporation of CKP-C16 into NLPs. Standard curves for each component were generated and used to calculate the amount of CKP-C16 incorporated into the purified CKP-NLPs. For the 10 mol % CKP-C16 samples, the incorporation of CKP-C16 was calculated to be 8.4 mol %, which indicates that there was a slight loss in material during the assembly and purification process. In addition to HPLC analysis of the CKP-NLP composition, NLP molecular weight was also measured by SEC-MALS (FIGS. 10D-10E). The MW of the CKP-NLP peak varied from 200 to 400 kDa with an average MW of 266 kDa across the peak (retention time 9-10 min) (FIG. 10D). Similarly, the hydrodynamic size (Rₕ) of the CKP-NLP peak varied from 5.8-7.3 nm with an average Rₕ of 6.2 nm across the peak (retention time 9-10 min) (FIG. 10E). These combined results suggest that the CKP-NLP particles are polydisperse. These findings are consistent with previous reports evaluating NLP size from both bulk and single molecule measurements. See, e.g., reference 6 listed below. Reference 6 reports that the polydisperse properties of NLPs were found to be driven by multiple discrete NLP sizes that were dictated by the number of apoE422k scaffold proteins associated with the NLP, which can vary from 4-7, and that the NLP polydispersity represented a stochastic Gaussian distribution of these discrete sizes. Based on the SEC analysis, the retention time, Rh and MW are consistent with NLPs containing on average 4 apoE422k/NLP. These combined findings demonstrate that CKP-NLPs could successfully be generated, purified and characterized, as exemplified by a model lipidated peptide, CKP-C16.

### Example 8: Analysis of the Effects of CKP Incorporation on NLP Size, Loading Efficiency, and CKP Activity

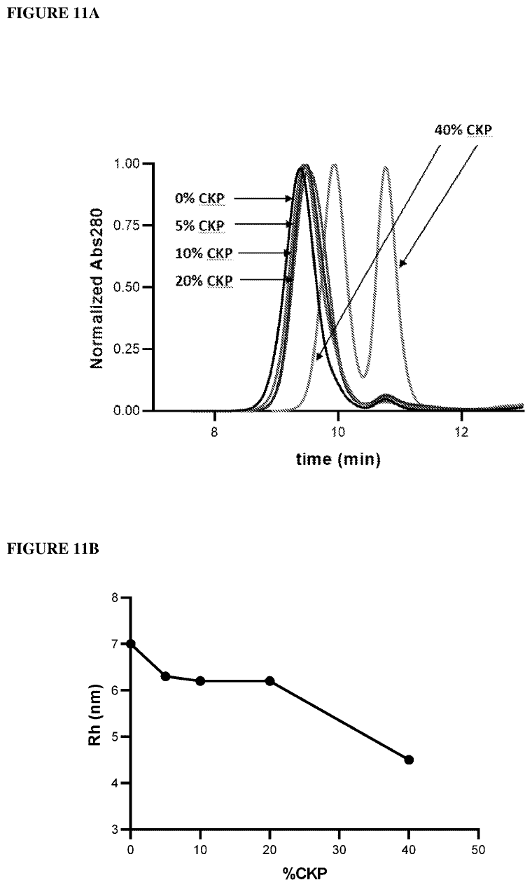

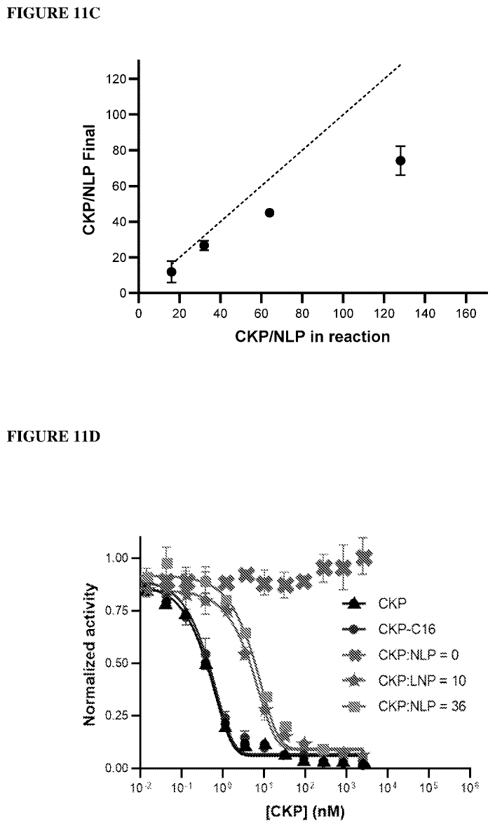

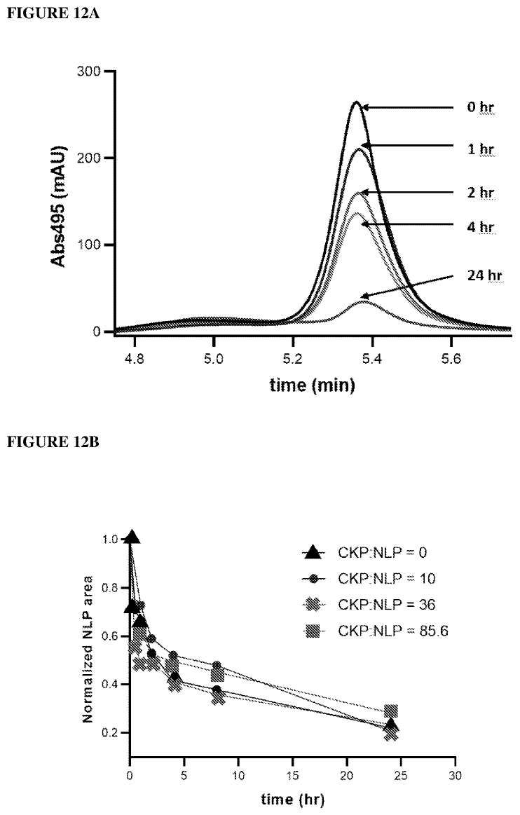

Next, the effect of CKP incorporation on NLP size was evaluated as follows. Maximal CKP loading was measured by assembling CKP-NLPs with CKP-C16 mol % ranging from 0-40% in the precursor organic phase. Normalized analytical SEC chromatograms for CKP-NLPs assembled with 0, 5%, 10%, 20% and 40% CKP-C16 are shown in FIG. 11A. As the CKP content was increased from 0-20%, a minor increase in retention time was observed, indicating a slight decrease in particle size. When the CKP-C16 concentration was increased from 20% to 40%, a large increase in retention time was observed as well as the emergence of a large peak corresponding to free apoE422k protein. These results are also reflected in the Rₕ analysis of the CKP-NLP peaks in SEC-MALS traces (FIG. 11B). The Rₕ of the NLP in the absence of CKP was ˜7 nm and decreased to ˜6.3 nm at 5% CKP. No further change in Rₕ was observed as the CKP-C16 content was increased from 5% to 20%; however, a drastic drop to 4.5 nm was observed when the CKP-C16 concentration was increased to 40%. As mentioned above, it was previously reported (see reference 6 below) that the size distribution of NLPs is largely dictated by a stochastic Gaussian distribution of discrete sized NLPs where the discrete size is determined by the number of apoE422k/NLP. Based on empirical single molecule measurements and molecular dynamic simulations, the addition of an apoE422k protein to the NLP results in ˜2.5 nm increase in diameter (see reference 6 below). Without being bound by theory, the ˜0.7 nm decrease in diameter observed when the CKP-C16 content was increased from 0 to 20% suggests that this change was not due to a difference in the average number of apoE422k/NLP. It has also been reported for a given discretely sized NLP there is some degree of flexibility in NLP size that can be dictated by composition (see reference 6 below). Without being bound by theory, this slight decrease in NLP size may be due to the changed NLP composition. CKP-C16 contains only a single fatty acyl chain and has a smaller surface area than DOPC, and this slight decrease in NLP diameter with increasing CKP-C16 content may be due to the smaller cross-sectional surface area of CKP-C16 relative to DOPC. This rationale may not explain the rapid drop in Rₕ and the emergence of the free apoE422k peak observed at the 40% CKP-C16 composition. This change in diameter closely matches the expected change when the average number of apoE422k/NLP is decreased by one (see reference 6 below). These findings suggest that when the CKP-C16 content is increased to 40%, there is no longer enough lipid content/surface area to accommodate 4 apoE422k/NLP that was observed for NLPs with the lower CKP-C16 content and that the particles consist of 3 apoE422k/NLP. However, if this were the case, the free apoE422k would be expected to increase by 25%, rather than the 50% increase observed based on area under the curve in the SEC chromatograms. An alternative explanation is that CKP-NLPs may no longer forming, and the CKP-C16-DOPC mixture may be forming spherical micelles. Mixing bilayer-forming lipids with detergents at higher detergent ratios can result in the formation of micelles (see reference 26 below). A similar phenomenon may be occurring with CKP-NLPs at higher CKP-C16 concentrations. Since CKP-C16 has a similar structure as a detergent molecule with a larger polar head group and single fatty acyl chain, it higher ratios CKP-C16 may induce the formation of micelles rather than bilayers, which is a requirement for the formation of NLPs. At at higher CKP molar ratios the fundamental structure and biophysical properties of the NLP are being altered.

To assess efficiency with which CKPs are incorporated into the NLP platform, the amount of CKP incorporated into the purified CKP-NLPs was quantified by reversed-phase HPLC. FIG. 11C shows the CKP/NLP ratios that were quantified in the purified CKP-NLPs (Y axis) as a function of the CKP content added during CKP-NLP assembly (X-axis), assuming 4 apoE422k/NLP. The dashed line represents the theoretical limit if all CKP in the reaction were incorporated into the NLP. At lower CKP concentrations (16 and 32 CKP/NLP, corresponding to 5 and 10 mol % CKP, respectively), almost all CKP added to the reaction was incorporated (13.3 and 27.6 CKP/NLP, respectively) as evidenced by how close the points were to the dashed lines (theoretical limit). However, at higher concentrations the CKP loading efficiency begins to deviate from the theoretical upper limit (FIG. 11C). Without being bound by theory, these findings suggest that there are diminishing returns in loading efficiency at higher CKP concentrations. The NLP was able to accommodate up to ˜60 CKP-C16 molecules without affecting the NLP assembly process. (FIG. 11C). To arrive at this conclusion, it was assumed that there were 4 apoE422k/NLP at the 40% CKP concentration (128 CKP/NLP). However, his may not be the case as higher CKP concentrations alter the number of apoE422k/NLP or induce the formation of micelle/NLP hybrids. Despite this potential discrepancy, these combined results suggest that there is a decrease in the incorporation efficiency at higher CKP loading conditions.

The effect of CKP incorporation into CKP-NLPs on CKP activity was evaluated as follows. For this analysis, CKP activity was measured at varying concentrations for free CKP, CKP-C16 and CKP-NLPs at varying CKP:NLP molar ratios (0, 13.4, 28.6 and 63) using a trypsin based assay (FIG. 11D). Although NLP-CKP assembly is relatively reproducible, variability between final compositions is typically observed. The CKP:NLP molar ratios for the present preparations were slightly different than the samples described above. Free CKP (triangles, FIG. 11D) and CKP-C16 (circles, FIG. 11D) molecules had equal potency with near equivalent Kᵢ^{app} (0.16) and the CKP-NLP sample with 0 CKP had no activity (crosses, FIG. 11D). The CKP-NLPs also had near equivalent potencies regardless of CKP loading density (stars and squares, FIG. 11D). However, the overall potency of the CKP in the CKP-NLP was about 5 fold less than free CKP and CKP-C16. Without being bound by theory, this may be due to (Kᵢ^{app}˜0.4 nM) or steric hindrance effects from having the CKP anchored to the NLP surface. It is well known that immobilization of an active ligand can affect activity because of steric effects (see references 27, 28 below). Alternatively, the CKP-C16 may undergo a phase separation process into clusters due to the chemical potential differences between the lipids and CKP-C16, and these clusters negatively affect CKP activity. Despite this negative impact on activity, the CKP was still highly potent when loaded on the NLP platform.

### Example 9: Effect of CKP Loading on CKP-NLP Stability

One of the caveats of NLP as a drug delivery platform is the low stability in complex biological milieu (see references 9, 24, 25 below). For example, the half-life of DOPC NLPs in 50% serum at 37° C. was reported to be between 3-6 hours (see reference 9 below), which is lower than the potential systemic half-life of nanoparticles (24-48 hours). It was recently demonstrated that crosslinking the bilayer core can significantly improve stability (see reference 24 below), where no NLP degradation was observed over a 48-hour time period in 100% serum for cross-linked NLPs and very rapid degradation (˜10 min) was observed in the absence of crosslinking (see reference 24 below). However, this approach may be associated with increased safety risk due to the addition of the non-natural cross-linked lipid, which has no known natural degradation or biotransformation pathway. It was recently reported that conjugation of Fabs to the NLP platform was reported to dramatically improve NLP stability (see reference 18 below). The effect of CKP incorporation on CKP NLP stability was evaluated as follows. The stability of CKP-NLPs was assessed in 50% serum using fluorophore tags to monitor degradation as a function of time by SEC. 50% serum was selected to mimic in vivo conditions. In these studies, AF488 labeled CKP-NLPs were injected onto an SEC at different time intervals and CKP-NLP elution was monitored at an absorbance of 495 nm. This fluorophore and absorbance signature was chosen for its non-overlapping spectrophotometric signature with the intrinsic absorbance of serum proteins and constituents in the serum/CKP-NLP sample. FIG. 12A shows SEC chromatograms of the naked NLP with no CKP at different time points. The first peak observed in the SEC chromatograms at a retention time of 5.25 min corresponded to the CKP-NLP and the third peak at a retention time of 7.1 min corresponded to the free apoE422k. As the incubation time was increased, a decrease in the CKP-NLP peak and increase in free apoE422k peak was observed. The decrease in NLP peak is a direct measure of NLP degradation and was quantified by calculating the area under the curve. In all samples, a middle peak at a retention time of 6.2 min was observed, and this peak did not change with incubation time. Given the consistency of this peak across all samples and time points, peak was likely due to a background contaminant in the serum.

To systematically evaluate the effect of CKP loading on stability, these experiments were performed with CKP-NLPs containing varying amounts of CKP-C16 (0, 10, and 36 CKP/NLP). The CKP-NLP SEC peak areas were normalized relative to peak area at time 0 h to allow for comparison across the different CKP-NLPs. In contrast to the effect reported for Fab conjugation to the NLP, the incorporation of CKP-C16 appeared to have no impact on overall NLP stability (FIG. 12B). All CKP-NLP samples displayed an initial rapid degradation with only 50% of the material remaining after 2-4 hours at 37° C., followed by slower degradation over the remaining 24 hour incubation period. This degradation pattern is very consistent with previously reports on NLPs (see references 18, 24 below). These findings suggest that the stabilizing effect previously observed for the Fab conjugation to the NLP surface, as described in Example 5, does not translate to molecules of a smaller size (Fab is ˜45 kDa and CKP is ˜3 kDa). Without being bound by theory, the Fabs could form a physical barrier that prevents interactions with serum proteins, which can destabilize amphipathic nanoparticles (Darwish M, Shatz W, Leonard B, Loyet K, Barrett K, Wong J L, Li H, Abraham R, Lin M, Franke Y, Tam C, Mortara K, Zilberleyb I, Blanchette CD, “Nanolipoprotein Particles as a Delivery Platform for Fab Based Therapeutics” Bioconjugate Chemistry, 2020, 31, 8 1995-2007). It was previously reported that a Fab mole loading of 7 was required before observing a stabilizing effect of Fab conjugation, which corresponds to ˜315 kDa of added MW to the NLP surface. By contrast, the highest CKP loading of 86 CKP/NLP only constituted an additional MW of ˜260 kDa. Therefore, if the increased stabilization due to Fab conjugation is primarily driven by added MW to the NLP surface, the amount of MW needed to stabilize that NLP was not reached even at the highest CKP loading. This may explain why no stabilization effect was observed for CKP incorporation. Alternatively, the stabilization effect results not only from added MW but also the hydrodynamic size of the conjugated entity. It is possible that no stabilization effect would have been observed even if it were possible to load more CKP on the surface without disrupting the overall NLP structure. These combined findings suggest that there will be limitations to circulating half-life and stability for in vivo delivery of CKPs and these limitations must be considered when applying this technology for disease treatment.

### Example 10: Assembly, Purification and Characterization of Fab-CKP-NLP Conjugates

Incorporation of both a CKP and a Fab into an NLP would permit the development of a nanoparticle platform that with more than one functional activity. For example, the NLP may comprise a Fab that exhibits targeting activity (e.g., HER2, CD20, etc.) and a CKP that exhibits therapeutic activity. Alternatively, an NLP may comprise a Fab and a CKP that exhibit synergistic therapeutic activities or complementary therapeutic activities. The potential for incorporating both CKP and Fab in the NLP was evaluated as follows. The strategy developed for the incorporation of both CKPs and Fabs into the NLP platform is described in FIG. 13A. The same method of CKP-NLP assembly was employed with the addition of a functionalized PEG lipid for subsequent conjugation to a Fab containing a functional reactive tag (FIG. 13A). The maleimide-thiol bioconjugation strategy, wherein the PEG lipid head group had a terminal reactive maleimide and the Fab contained an C-terminal cysteine amino acid, was used to conjugate the Fab to the NLP (see reference 18 below). A strategy for conjugation of Fab via thiol-maleimide chemistry using a DOPE lipid functionalized with a maleimidomethyl cyclohexane-1-carboxylate (DOPE-MCC) head group is described in Example 1. Initial pilot NLP assemblies were performed with the DOPE-MCC lipid and precipitation was consistently observed when CKP-C16 was added to the lipid mixture, which was not observed when DSPE-PEG-Mal was used. Therefore, DSPE-PEG-Mal was used for further optimization of Fab conjugation to the NLP platform. CKP-NLPs were assembled with 10% mol DSPE-PEG-Mal lipid to ensure sufficient maleimide ligand density on the NLP surface for Fab conjugation. A human anti-Factor D Fab containing a C-terminal cysteine was used as a surrogate Fab since this molecule was previously successfully conjugated to a PEG polymer scaffold (see reference 22 below) and NLP (see reference 18 below) via thiol-maleimide conjugation. Fabs were conjugated to the maleimide functionalized CKP-NLP directly after the biobead-based cholate removal step without a CKP-NLP purification step. This approach was used to minimize the potential for maleimide hydrolysis prior to conjugation. The initial pilot conjugation was performed at a Fab:CKP-NLP molar ratio of 20. Fabs were incubated with the CKP-NLP for 2-3 hours and NAC was added at 2-fold molar concentration over DSPE-PEG-Mal to quench unreacted maleimides and prevent DOPE-MCC:apoE422k crosslinking as described previously (see reference 16 below). Fab-CKP-NLPs were purified by SEC and three main peaks were observed in the SEC chromatograms; Fab-CKP-NLP conjugate (rt ˜10 min), Fab dimer (rt ˜14 min) and unconjugated Fab (rt ˜16 min) (FIG. 13B). The presence of the Fab dimer after the conjugation reaction was due to dimerization via disulfide formation at the C-terminal thiols of the Fabs during the conjugation step, as described previously (see reference 18 below). Fractions across the center of the NLP peak were collected, pooled and subjected to HPLC and SEC-MALS analysis. An exemplary HPLC chromatogram of a purified and reduced Fab-CKP-NLP sample is shown in FIG. 13C. Peaks were observed at retention times of 1.75, 2.05, 2.8, 3.1, 3.4 and 3.95 min and corresponded to Fab light chain, DSPE-PEG-Mal, apoE422k, CKP-C16, Fab heavy chain-DSPE-PEG-Mal conjugate, and DOPC, respectively. Standard curves for each component were generated and used to quantify CKP-C16 incorporation and Fab conjugation. The exemplary HPLC chromatogram shown in FIG. 13C corresponds to 18 CKP/NLP and 23 Fab/NLP. As was described for the CKP-NLPs, particle size was also measured by SEC-MALS (FIG. 13D-13E). The variability in the MW of the Fab-CKP-NLP peak (220 kDa to 1500 kDa, with an average MW of 700 kDa) was significantly broader than the CKP-NLP (200 to 400 kDa, with an average MW of 266 kDa across the peak) (FIG. 13D). A similar trend in the variability of the Rₕ across the Fab-CKP-NLP peak was also observed. Without being bound by theory, the increased variability in MW and Rh for the Fab-CKP-NLP may be due to the distribution in number of conjugated Fabs/NLP. Based on the HPLC analysis of this Fab-CKP-NLP sample, there were on average 20 Fabs/NLP; however, the distribution of Fab/NLP will be Gaussian and we would expect to see CKP-NLPs with Fabs varying from 0 (MW ˜300 kDa) up to 30 (MW ˜1500 kDa). The variability in Rₕ across the Fab-CKP-NLP peak (FIG. 13E) was similar to the CKP-NLP (˜2 fold) (FIG. 10E). It was previously reported that conjugation of Fabs and protein to the NLP had significantly less impact on Rₕ than MW because the size of the discoidal NLP is largely driven by diameter and not bilayer height, which is most impacted by protein conjugation to the NLP surface (see reference 18 below). These findings are consistent with this hypothesis.

### Example 11: Effects of CKP and Fab Loading on Maximal Fab Conjugation, Fab-CKP-NLP Size, CKP Activity and Fab-CKP-NLP Stability

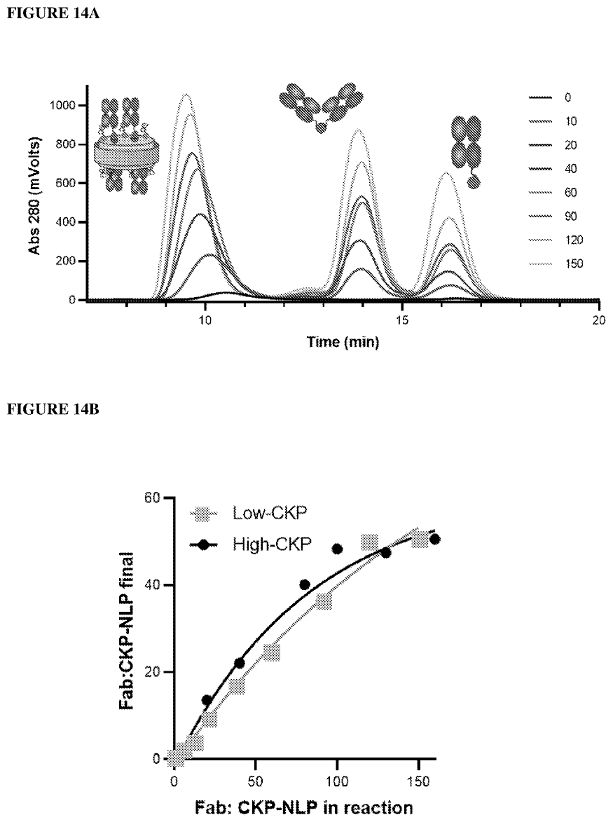

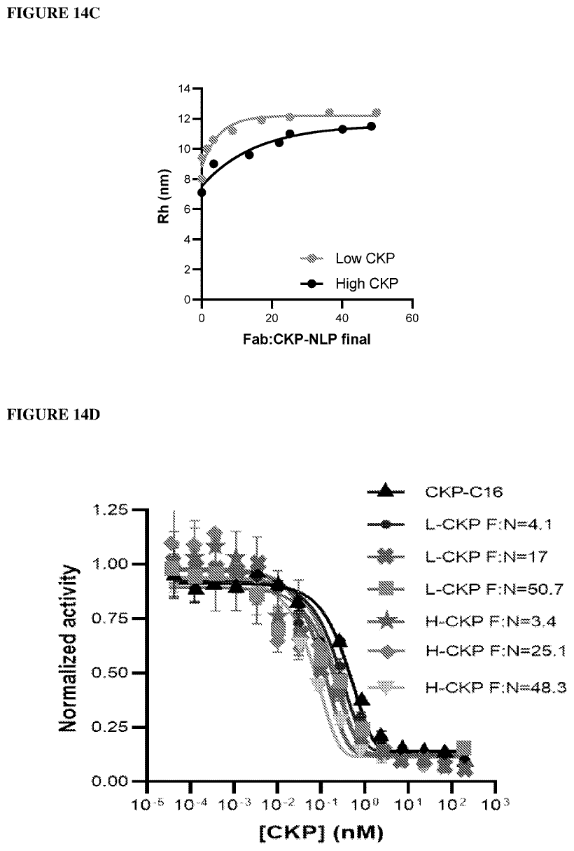

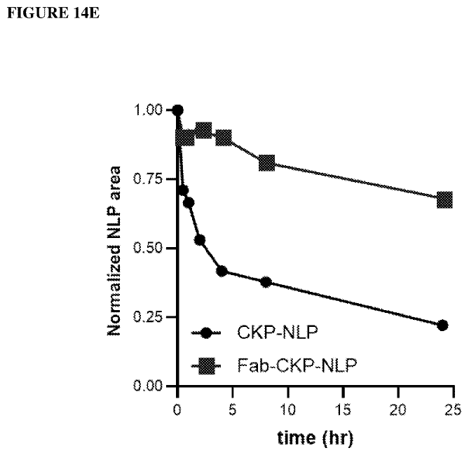

The impact of CKP loading density and Fab loading density on Fab-CKP-NLP size, activity and serum stability were examined as follows. CKP-NLPs were assembled with 10 mol % DOPE-PEG-Mal at two CKP loading densities, 12.7 CKP/NLP (referred to as “Low CKP-NLP”) and 40 CKP/NLP (referred to as “High CKP-NLP”) and conjugated to the Fab at different Fab to CKP-NLP ratios. FIG. 14A shows the SEC chromatograms of the Low CKP-NLP conjugated to Fab at Fab to CKP-NLP ratios ranging from 0 to 150. The highest Fab:NLP ratio of 150 corresponded to ˜1 Fab: 1 DSPE-PEG-Mal. The CKP-NLP peak gradually increased with an increase in Fab concentration up to a saturation level at ˜150. A gradual increase in the Fab dimer and unconjugated Fab peak was observed with an increase in the Fab conjugation ratio. This was repeated for the High CKP-NLP and the amount of Fab conjugated was quantified as described in Example 6 (FIG. 14B). For both CKP compositions, a linear increase in conjugated Fab loading (amount actually attached to the NLP) was observed when the Fab:NLP ratio in the reaction was increased from 0-100. However, minimal to no increase in Fab loading was observed at higher ratios and the saturating amount of Fab conjugated to the NLP surface was determined to be ˜50 Fab/NLP (FIG. 14B). By contrast, the maximum Fab loading capacity was previously reported to be 30 when conjugated directly to the NLP surface through the DOPE-MCC lipid at a 10 mol % DOPE-MCC-lipid composition. This maximum loading capacity was suggested to be driven by the available NLP surface area, and there was a strong correlation between the NLP surface area and overall surface area occupied by 30 Fab. The surface area of the CKP-NLP was similar to that described in Darwish M, Shatz W, Leonard B, Loyet K, Barrett K, Wong J L, Li H, Abraham R, Lin M, Franke Y, Tam C, Mortara K, Zilberleyb I, Blanchette C D, “Nanolipoprotein Particles as a Delivery Platform for Fab Based Therapeutics” Bioconjugate Chemistry, 2020, 31, 8 1995-2007, and therefore the increased Fab loading may not be attributable to an increased surface area. A similar increased Fab binding capacity in the absence of the CKPs was observed, suggesting that this effect was not due to the presence of the CKP on the NLP surface. In this study, the Fabs were not conjugated directly to the surface but rather to a PEG2000 linker extending from the NLP surface. Therefore, it is plausible that the spacer increased the overall conjugation volume and limited steric hindrance allowing for a higher number of Fabs to conjugate to the NLP when compared to direct conjugation to the NLP surface. The CKP density had no appreciable impact on Fab loading (FIG. 14B). Thus, this result was unexpected given the potential for greater steric hindrance at the higher CKP loading. Without being bound by theory, the PEG linker may eliminate any steric impact from the CKP since it creates a spacer between the NLP surface and the conjugation handle for Fab loading.

To further evaluate the impact of Fab loading on NLP size, the Rₕ was measured by SEC-MALS at the different Fab loadings for both the High and Low CKP NLPs (FIG. 14C). An increase in Rh was observed when the Fab loading was increased from 0 to 10 Fab/NLP and only minimal increases were observed as the Fab loading was increased from 10 to 50 Fab/NLP. These results are consistent with previous findings (see references 9, 11, 18 below) and suggest that Fab conjugation does not alter the discoidal nature of the NLP and that NLP diameter and surface area, rather than Fab loading, are the drivers of Rh.

Conjugation of the Fab to the NLP surface may create a potential physical barrier for analyte interaction with the CKP. Therefore, to evaluate the impact of Fab loading on CKP activity, the CKP activity in both Low and High CKP-NLPs with varying Fab densities was evaluated (FIG. 14D). The Fab density had no impact on CKP activity for both the High and Low CKP-NLP formulations and the difference in substrate inhibition constant (Kᵢ^{app}) between the samples was within the error of the assay (0.3-0.05 nM). By contrast, and as discussed in Example 8, a decrease in CKP activity was observed in the absence of Fab conjugation These unexpected results observed in Fab-CKP-NKPs were highly reproducible. These results indicate that Fab loading can be reliably controlled from 1-50 Fab/CKP-NLP, that the degree of Fab loading is not dependent on CKP density, and that Fab conjugation does not appear impact on CKP activity, regardless of Fab density. Without being bound by theory, it is possible that the Fabs are nonspecifically binding to trypsin, increasing the effective trypsin concentration at the NLP surface. It is also plausible that if CKP-C16 are phase separating into patches resulting in decreased activity in the absence of Fab conjugation that this effect is mitigated after Fab conjugation resulting in rescued activity.

As described in Example 5, Fab conjugation can have a dramatic impact on NLP stability, possibly by providing a steric barrier against serum proteins. To determine whether these findings can extend to the CKP-NLP platform, the stability of Fab-CKP-NLPs (50 Fab/NLP and 20 CKP/NLP) were compared to CKP-NLPs (20 CKP/NLP) in 50% serum (FIG. 14E). As has been observed for “empty” NLPs (i.e., NLPs that have not been loaded with CKP and/or Fab), Fab conjugation had a significant impact on CKP-NLP stability. The CKP-NLPs rapidly degraded and within the first 4 h more than 50% of the particles had degraded. By contrast, the Fab-CKP-NLPs were surprisingly stable and more than 75% of the material remained intact over 24 h. Without being bound by theory, these findings suggest that the stabilizing effect of the Fab layer is maintained even when the Fab is attached via a PEG spacer and in the presence of additional molecular peptidic entities on the NLP surface.

### Conclusions from Examples 7-11

Examples 7-11 describe the development and characterization of both CKP-NLPs and Fab-CKP-NLP conjugates. For CKP incorporation, self-assembly strategy was developed where the CKP was appended with a C16 hydrocarbon tail, enabling partitioning into the bilayer core during the assembly process and displaying the CKP on the NLP surface. The NLP was able to accommodate up to ˜60 CKP-C16 molecules without affecting the NLP assembly process. CKP-C16 incorporation into the NLP slightly decreased CKP trypsin inhibitory activity, but the molecule was still highly potent (sub-nano molar binders). The CKP-NLP stability was found to be comparable to the NLP alone and had a relatively short half-life in 50% serum at 37° C. (˜1 h). Fab conjugation to the NLP platform was achieved by introducing a thiol reactive maleimide lipid where the maleimide group was attached to a PEG spacer. Using this conjugation strategy, Fab loading could be reliably controlled from 1-50 Fab/CKP-NLP and the degree of Fab loading was not dependent on CKP density. Fab conjugation also did not have an impact on CKP activity regardless of Fab density. Finally. Fab conjugation had a profound impact on improving CKP-NLP stability and more than 75% of the Fab-CKP-NLP remained intact after incubation at 37° C. for 24 h. These combined findings suggest that NLPs are a promising platform for potential delivery of peptide-like drug candidates. Moreover, targeted or multidrug delivery could be achieved through conjugation of a targeting or therapeutic Fab to the CKP-NLP.

### REFERENCES FOR EXAMPLES 6-11

(1) Ackerman, S. E., Currier, N. V., Bergen, J. M., and Cochran, J. R. (2014) Cystine-knot peptides: emerging tools for cancer imaging and therapy. Expert Rev Proteomics 11, 561-72.
(2) Gao, X., Stanger, K., Kaluarachchi, H., Maurer, T., Ciepla, P., Chalouni, C., Franke, Y., and Hannoush, R. N. (2016) Cellular uptake of a cystine-knot peptide and modulation of its intracellular trafficking. Sci Rep 6, 35179.
(3) Reinwarth, M., Nasu, D., Kolmar, H., and Avrutina, O. (2012) Chemical synthesis, backbone cyclization and oxidative folding of cystine-knot peptides: promising scaffolds for applications in drug design. Molecules 17, 12533-52.
(4) Bazak, R., Houri, M., El Achy, S., Kamel, S., and Refaat, T. (2015) Cancer active targeting by nanoparticles: a comprehensive review of literature. J Cancer Res Clin Oncol 141, 769-84.
(5) Fischer, N. O., Blanchette, C. D., Segelke, B. W., Corzett, M., Chromy, B. A., Kuhn, E. A., Bench, G., and Hoeprich, P. D. (2010) Isolation, characterization, and stability of discretely-sized nanolipoprotein particles assembled with apolipophorin-III. PLoS One 5, e11643.
(6) Blanchette, C. D., Law, R., Benner, W. H., Pesavento, J. B., Cappuccio, J. A., Walsworth, V., Kuhn, E. A., Corzett, M., Chromy, B. A., Segelke, B. W., Coleman, M. A., Bench, G., Hoeprich, P. D., and Sulchek, T. A. (2008) Quantifying size distributions of nanolipoprotein particles with single-particle analysis and molecular dynamic simulations. J Lipid Res 49, 1420-30.
(7) Martinez, D., Decossas, M., Kowal, J., Frey, L., Stahlberg, H., Dufourc, E. J., Riek, R., Habenstein, B., Bibow, S., and Loquet, A. (2017) Lipid Internal Dynamics Probed in Nanodiscs. Chemphyschem 18, 2651-2657.
(8) Mors, K., Roos, C., Scholz, F., Wachtveitl, J., Dotsch, V., Bernhard, F., and Glaubitz, C. (2013) Modified lipid and protein dynamics in nanodiscs. Biochim Biophys Acta 1828, 1222-9.
(9) Fischer, N. O., Weilhammer, D. R., Dunkle, A., Thomas, C., Hwang, M., Corzett, M., Lychak, C., Mayer, W., Urbin, S., Collette, N., Chiun Chang, J., Loots, G. G., Rasley, A., and Blanchette, C. D. (2014) Evaluation of nanolipoprotein particles (NLPs) as an in vivo delivery platform. PLoS One 9, e93342.
(10) Fischer, N. O., Rasley, A., Corzett, M., Hwang, M. H., Hoeprich, P. D., and Blanchette, C. D. (2013) Colocalized delivery of adjuvant and antigen using nanolipoprotein particles enhances the immune response to recombinant antigens. J Am Chem Soc 135, 2044-7.
(11) Blanchette, C. D., Fischer, N. O., Corzett, M., Bench, G., and Hoeprich, P. D. (2010) Kinetic analysis of his-tagged protein binding to nickel-chelating nanolipoprotein particles. Bioconjug Chem 21, 1321-30.
(12) He, W., Felderman, M., Evans, A. C., Geng, J., Homan, D., Bourguet, F., Fischer, N. O., Li, Y., Lam, K. S., Noy, A., Xing, L., Cheng, R. H., Rasley, A., Blanchette, C. D., Kanrud, K., Wang, N., Gouvis, H., Peterson, T. C., Hubby, B., and Coleman, M. A. (2017) Cell-free production of a functional oligomeric form of a Chlamydia major outer-membrane protein (MOMP) for vaccine development. J Biol Chem 292, 15121-15132.
(13) Tufteland, M., Pesavento, J. B., Bermingham, R. L., Hoeprich, P. D., Jr., and Ryan, R. O. (2007) Peptide stabilized amphotericin B nanodisks. Peptides 28, 741-6.
(14) Jia, J., Xiao, Y., Liu, J., Zhang, W., He, H., Chen, L., and Zhang, M. (2012) Preparation, characterizations, and in vitro metabolic processes of paclitaxel-loaded discoidal recombinant high-density lipoproteins. J Pharm Sci 101, 2900-8.
(15) Weilhammer, D., Dunkle, A. D., Blanchette, C. D., Fischer, N. O., Corzett, M., Lehmann, D., Boone, T., Hoeprich, P., Driks, A., and Rasley, A. (2017) Enhancement of antigen-specific CD4(+) and CD8(+) T cell responses using a self-assembled biologic nanolipoprotein particle vaccine. Vaccine 35, 1475-1481.
(16) Kuai, R., Sun, X., Yuan, W., Xu, Y., Schwendeman, A., and Moon, J. J. (2018) Subcutaneous Nanodisc Vaccination with Neoantigens for Combination Cancer Immunotherapy. Bioconjug Chem 29, 771-775.
(17) Weilhammer, D. R., Blanchette, C. D., Fischer, N. O., Alam, S., Loots, G. G., Corzett, M., Thomas, C., Lychak, C., Dunkle, A. D., Ruitenberg, J. J., Ghanekar, S. A., Sant, A. J., and Rasley, A. (2013) The use of nanolipoprotein particles to enhance the immunostimulatory properties of innate immune agonists against lethal influenza challenge. Biomaterials 34, 10305-18.
(18) Darwish, M., Shatz, W., Leonard, B., Loyet, K., Barrett, K., Wong, J. L., Li, H., Abraham, R., Lin, M., Franke, Y., Tam, C., Mortara, K., Zilberleyb, I., and Blanchette, C. (2020) Nanolipoprotein Particles as a Delivery Platform for Fab Based Therapeutics. Bioconjug Chem 31, 1995-2007.
(19) Fischer, N. O., Blanchette, C. D., Chromy, B. A., Kuhn, E. A., Segelke, B. W., Corzett, M., Bench, G., Mason, P. W., and Hoeprich, P. D. (2009) Immobilization of His-tagged proteins on nickel-chelating nanolipoprotein particles. Bioconjug Chem 20, 460-5.
(20) Zhang, Y. N., Appleton, B. A., Wiesmann, C., Lau, T., Costa, M., Hannoush, R. N., and Sidhu, S. S. (2009) Inhibition of Wnt signaling by Dishevelled PDZ peptides. Nat Chem Biol 5, 217-219.
(21) Fischer, N. O., Infante, E., Ishikawa, T., Blanchette, C. D., Bourne, N., Hoeprich, P. D., and Mason, P. W. (2010) Conjugation to nickel-chelating nanolipoprotein particles increases the potency and efficacy of subunit vaccines to prevent West Nile encephalitis. Bioconjug Chem 21, 1018-22.
(22) Shatz, W., Hass, P. E., Peer, N., Paluch, M. T., Blanchette, C., Han, G., Sandoval, W., Morando, A., Loyet, K. M., Bantseev, V., Booler, H., Crowell, S., Kamath, A., Scheer, J. M., and Kelley, R. F. (2019) Identification and characterization of an octameric PEG-protein conjugate system for intravitreal long-acting delivery to the back of the eye. PLoS One 14, e0218613.
(23) Gao, T., Blanchette, C. D., He, W., Bourguet, F., Ly, S., Katzen, F., Kudlicki, W. A., Henderson, P. T., Laurence, T. A., Huser, T., and Coleman, M. A. (2011) Characterizing diffusion dynamics of a membrane protein associated with nanolipoproteins using fluorescence correlation spectroscopy. Protein Sci 20, 437-47.
(24) Gilmore, S. F., Blanchette, C. D., Scharadin, T. M., Hura, G. L., Rasley, A., Corzett, M., Pan, C. X., Fischer, N. O., and Henderson, P. T. (2016) Lipid Cross-Linking of Nanolipoprotein Particles Substantially Enhances Serum Stability and Cellular Uptake. ACS Appl Mater Interfaces 8, 20549-57.
(25) Gilmore, S. F., Carpenter, T. S., Ingolfsson, H. I., Peters, S. K. G., Henderson, P. T., Blanchette, C. D., and Fischer, N. O. (2018) Lipid composition dictates serum stability of reconstituted high-density lipoproteins: implications for in vivo applications. Nanoscale 10, 7420-7430.
(26) Lichtenberg, D., Ahyayauch, H., and Goni, F. M. (2013) The mechanism of detergent solubilization of lipid bilayers. Biophys J 105, 289-99.
(27) Shuai, W., Das, R. K., Naghdi, M., Brar, S. K., and Verma, M. (2017) A review on the important aspects of lipase immobilization on nanomaterials. Biotechnol Appl Biochem 64, 496-508.
(28) Bagheri, M., Beyermann, M., and Dathe, M. (2009) Immobilization reduces the activity of surface-bound cationic antimicrobial peptides with no influence upon the activity spectrum. Antimicrob Agents Chemother 53, 1132-41.

1. A conjugate comprising:
a scaffold protein, a membrane-forming lipid, and an antigen-binding polypeptide,
wherein said membrane-forming lipids arrange in a lipid bilayer and said scaffold protein encircles said bilayer; and
wherein said antigen-binding polypeptide is conjugated to one or more of said membrane-forming lipids on one or both surfaces of said bilayer via a functionalized group on said one or more membrane-forming lipids and a complementary functional group located C-terminally on said antigen-binding polypeptide.

2-4. (canceled)

5. The conjugate of claim 1, wherein said scaffold protein is selected from the group consisting of (a) apolipoprotein A, (b) apolipoprotein B, (c) apolipoprotein C, (d) apolipoprotein D, (e) apolipoprotein H, (f) apolipoprotein E, (g) a truncated version of (a)-(f) capable of stabilizing said bilayer, and (h) a combination of any one or more of (a)-(g).

6. The conjugate of claim 1, wherein said membrane-forming lipids are selected from the group consisting of C₄₋₂₈ fatty-acyl, DMPC, DOPC, DOPS, DOPE, DPPC, and a combination of any one or more thereof.

7. (canceled)

8. A conjugate comprising:
a scaffold protein, a membrane-forming lipid, and a short peptide of 20-60 amino acids,
wherein said membrane-forming lipids arrange in a lipid bilayer and said scaffold protein encircles said bilayer; and
wherein said short peptide is conjugated to one or more of said membrane-forming lipids on one or both surfaces of said bilayer.

9. The conjugate of claim 8, wherein said membrane-forming lipids are selected from the group consisting of C₄₋₂₈ fatty-acyl, DMPC, DOPC, DOPS, DOPE, DPPC, and a combination of any one or more thereof.

10-11. (canceled)

12. The conjugate of claim 8, wherein said short peptide is a cystine-knot peptide (CKP) or a CKP variant.

13-28. (canceled)

29. The conjugate of claim 1, wherein said antigen-binding polypeptide is selected from the group consisting of a Fab, a Fab′, a Fab′-SH, a F(ab′)₂, a single chain Fab (scFab), a single chain Fv (scFv), a VH-VH dimer, a VL-VL dimer, a VH-VL dimer, a single domain, a diabody, a linear antibody, and a combination of any one or more thereof, particularly wherein said antigen-binding polypeptide is a Fab.

30. (canceled)

31. The conjugate of claim 1, wherein said conjugate has 1-160, 10-120, 20-100, 40-80, or 60 molecules of said antigen-binding polypeptide;
wherein said antigen-binding polypeptide is a Fab and said Fab is connected by a PEG spacer to said membrane-forming lipid.

32. The conjugate of claim 31, wherein said PEG spacer connects said functionalized group to said membrane-forming lipid and has a MW of 1000-3000, 1500-2500, 1900-2200, or 2000.

33. The conjugate of claim 1, wherein said conjugate increases an activity or avidity of said antigen-binding polypeptide.

34. The conjugate of claim 33, where said activity is OX40 binding and said conjugate has 4-8 molecules of said antigen-binding polypeptide.

35. The conjugate of claim 1, wherein said antigen-binding polypeptide increases serum stability and/or serum half-life of said conjugate.

36. (canceled)

37. The conjugate of claim 1, wherein said two or more antigen-binding polypeptides bind different targets or epitopes.

38. The conjugate of claim 37, wherein said two or more antigen-binding polypeptides bind two different targets or epitopes.

39. The conjugate of claim 37, wherein said targets are a pair selected from the group consisting of CD3 and CD19; CD3 and EpCAM; CD3 and CEA; CD16 and CD30; CD16 and CD33; Ang-2 and VEGF-A; and Factor X and Factor IXa.

40. (canceled)

41. The conjugate of claim 1, wherein the spacer comprises PEG or the amino acid sequence of GSGS.

42. A pharmaceutical composition comprising the conjugate of claim 1 and a pharmaceutically acceptable vehicle.

43-47. (canceled)

48. A liquid formulation comprising 100-300 mg of the conjugate of claim 1 and having a viscosity of 10-50 cP.

49. A method of preparing a conjugate of a nanolipoprotein particle and an antigen-binding polypeptide, comprising:
a) providing a scaffold protein and a membrane-forming lipid under conditions allowing (self-)assembly of a nanolipoprotein particle comprising a lipid bilayer of said membrane-forming lipids encircled by said scaffold protein, wherein one or more of said membrane-forming lipids presents a functionalized group on one or both surfaces of said particle; and
b) allowing said particle to contact an antigen-binding polypeptide having a C-terminally located complementary functional group that conjugates to said functionalized group at low pH in the range of 4.5 to 6.5 pH.

50-74. (canceled)

75. A method of preparing a conjugate of a nanolipoprotein particle and a short peptide of 20-60 amino acids, comprising:
providing a scaffold protein and a membrane-forming lipid under conditions allowing (self-)assembly of a nanolipoprotein particle comprising a lipid bilayer of said membrane-forming lipids encircled by said scaffold protein; and
wherein one or more of said membrane-forming lipids comprises a C₄₋₂₈ fatty-acyl and fatty-acyl is conjugated to said short peptide.

76-100. (canceled)

101. A conjugate produced by the method of claim 49.

102. A method of increasing stability, shelf-life, half-life, avidity, activity, and/or potency of an antigen-binding polypeptide or a nanolipoprotein particle, said method comprising:
a) providing a scaffold protein and a membrane-forming lipid under conditions allowing (self-)assembly of a nanolipoprotein particle comprising a lipid bilayer of said membrane-forming lipids encircled by said scaffold protein, wherein one or more of said membrane-forming lipids presents a functionalized group on one or both surfaces of said particle; and
b) allowing said particle to contact an antigen-binding polypeptide having a C-terminally located complementary functional group that conjugates to said functionalized group at low pH in the range of 4.5 to 6.5 pH;
thereby increasing stability, shelf-life, half-life, avidity, activity, and/or potency of said antigen-binding polypeptide or said nanolipoprotein particle.

103-128. (canceled)

129. A method of increasing the stability, shelf-life, and/or half-life of a short peptide that is 20-60 amino acids in length, said method comprising:
a) providing a scaffold protein and a membrane-forming lipid under conditions allowing (self-)assembly of a nanolipoprotein particle comprising a lipid bilayer of said membrane-forming lipids encircled by said scaffold protein;
wherein one or more of said membrane-forming lipids comprises a C₄₋₂₈ fatty-acyl and fatty-acyl is conjugated to said short peptide; and
optionally purifying the peptide conjugate;
b) modifying one or more of said membrane-forming lipids to a present functionalized group on one or both surfaces of said particle, and
c) allowing said particle to contact an antigen-binding polypeptide having a C-terminally located complementary functional group that conjugates to said functionalized group at low pH in the range of 4.5 to 6.5 pH;
wherein said step c) follows said step b) without an intervening step to remove some or all unassembled membrane-forming lipids; and/or without an intervening step to enrich the peptide conjugate of step b)
thereby increasing stability, shelf-life, and/or half-life of said short peptide.

130-144. (canceled)

145. A method of treating an individual with an antigen-binding polypeptide, comprising administering to the individual an effective amount of the conjugate of claim 1.

146. A method of delivering an antigen-binding polypeptide to an individual in need thereof, said method comprising
providing the conjugate of claim 1; and
administering said conjugate, composition, or formulation to said individual, thereby delivering said antigen-binding polypeptide to said individual.

147. A method of delivering an antigen-binding polypeptide to an individual in need thereof, in a potent, stable and/or low viscosity liquid, formulation, said method comprising:
administering to an individual a liquid formulation comprising a conjugate of said antigen-binding polypeptide and a nanolipoprotein particle;
wherein said nanolipoprotein particle comprises a scaffold protein encircling a bilipid layer of membrane-forming lipids;
wherein said one or more membrane-forming lipids are conjugated to said antigen-binding polypeptide via a functionalized group on said one or more membrane-forming lipids and a C-terminally located complementary functional group on said antigen-binding polypeptide;
thereby delivering said antigen-binding polypeptide to an individual in a stable or low viscosity liquid formulation.

148-149. (canceled)

150. A method of delivering a short peptide 20-60 amino acids in length to an individual in need thereof, in a potent, stable and/or low viscosity liquid formulation, said method comprising:
administering to an individual a liquid formulation comprising a conjugate of said antigen-binding polypeptide and a nanolipoprotein particle;
wherein said nanolipoprotein particle comprises a scaffold protein encircling a bilipid layer of membrane-forming lipids;
wherein one or more of said membrane-forming lipids is a C₄₋₂₈ fatty-acyl and wherein said fatty-acyl is conjugated to a short peptide of 20-60 amino acids;
wherein one or more of said membrane-forming lipids are conjugated to said antigen-binding polypeptide via a functionalized group on said membrane-forming lipid and a C-terminally located complementary functional group on said antigen-binding polypeptide;
thereby delivering said antigen-binding polypeptide to an individual in a stable and/or low viscosity liquid formulation.

151-174. (canceled)

175. A method of delivering a biologically active agent or a detectable agent to an individual in need thereof, in a stable and/or low viscosity liquid formulation, said method comprising:
administering to an individual a liquid formulation comprising a conjugate of an antigen-binding polypeptide, a nanolipoprotein particle, and a biologically active agent or a detectable agent,
wherein said nanolipoprotein particle comprises a scaffold protein encircling a bilipid layer of membrane-forming lipids;
wherein one or more of said membrane-forming lipids are conjugated to said antigen-binding polypeptide via a functionalized group on said membrane-forming lipid and a C-terminally located complementary functional group on said antigen-binding polypeptide;
wherein said biologically active agent or said detectable agent is conjugated to at least one of said scaffold protein, said membrane-forming lipid, or said antigen-binding polypeptide;
thereby delivering said biologically active agent or said detectable agent to an individual in need thereof in a stable and/or low viscosity liquid formulation.

176. (canceled)

177. A method of delivering a biologically active agent or a detectable agent to the brain or central nervous system of an individual in need thereof, said method comprising:
systemically administering to an individual a liquid formulation comprising a conjugate of an antigen-binding polypeptide, a nanolipoprotein particle, and a biologically active agent or a detectable agent,
wherein said nanolipoprotein particle comprises a scaffold protein encircling a bilipid layer of membrane-forming lipids;
wherein one or more of said membrane-forming lipids is conjugated to said antigen-binding polypeptide via a functionalized group on said one or more membrane-forming lipids and a C-terminally located complementary functional group on said antigen-binding polypeptide;
wherein said biologically active agent or said detectable agent is conjugated to at least one of said scaffold protein, said membrane-forming lipid, or said antigen-binding polypeptide;
and
wherein said conjugate translocates across the blood-brain barrier,
thereby delivering said biologically active agent or said detectable agent to o the brain or central nervous system of an individual in need thereof.

178-197. (canceled)

198. A method of reducing lipid-scaffold protein conjugation in preparing a conjugate of a nanolipoprotein particle and an antigen-binding polypeptide, comprising:
a) providing a scaffold protein and a membrane-forming lipid under conditions allowing assembly of a nanolipoprotein particle comprising a lipid bilayer of said membrane-forming lipids encircled by said scaffold protein, wherein one or more of said membrane-forming lipids presents a functionalized group on one or both surfaces of said particle;
b) allowing said particle to contact an antigen-binding polypeptide having a C-terminally located complementary functional group at a low pH that favors conjugation of said functionalized group to said functional group rather than to said scaffold protein.

199-208. (canceled)

209. A method of increasing the biological activity of a short peptide attached to the surface of a lipid-based nanoparticle, comprising
(a) providing a lipid-based nanoparticle comprising a short peptide attached its surface, wherein one or more lipids of the lipid-based nanoparticle presents a functionalized group, and
(b) allowing the nanoparticle to contact a polypeptide having a functional group under conditions that favor conjugation of said functionalized group to said functional group.

210-217. (canceled)


SEQUENCE LISTING (1 sequence)

SEQ ID NO: 1 - protein, 28 aa, Ecballium elaterium
GCPRILMRCKQDSDCLAGCVCGPNGFCG